(19) United States
(12) Patent Application Publication
(10) Pub. No.: US 2024/0173317 A1
(43) Pub. Date: May 30, 2024

(54) METHODS FOR TREATING PROGRESSIVE FAMILIAL INTRAHEPATIC CHOLESTASIS

(71) Applicant: MIRUM PHARMACEUTICALS, INC., Foster City, CA (US)

(72) Inventor: Thomas JAECKLIN, Delemont (CH)

(73) Assignee: MIRUM PHARMACEUTICALS, INC., Foster City, CA (US)

(21) Appl. No.: 18/382,636

(22) Filed: Oct. 23, 2023

Related U.S. Application Data
(60) Provisional application No. 63/418,589, filed on Oct. 23, 2022.
(60) Provisional application No. 63/423,310, filed on Nov. 7, 2022.
(60) Provisional application No. 63/471,291, filed on Jun. 6, 2023.
(60) Provisional application No. 63/522,355, filed on Jun. 21, 2023.

(51) Int. Cl. A61K 31/4995; A61K 9/00; A61K 9/08; A61K 31/07; A61K 31/355; A61K 31/593; A61K 47/10; A61P 1/16

(52) U.S. Cl. CPC A61K 31/4995; A61K 9/0053; A61K 9/08; A61K 31/07; A61K 31/355; A61K 31/593; A61K 47/10; A61P 1/16

(57) ABSTRACT

Provided herein are methods for treating cholestasis in a subject having liver disease. More specifically, the present invention relates to methods for treating Progressive Familial Intrahepatic Cholestasis (PFIC) in a subject where the method includes administering maralixibat to a subject in need thereof.

### CROSS REFERENCE TO RELATED APPLICATIONS

This application claims benefit to Provisional Application Nos. 63/418,589, filed Oct. 23, 2022, 63/423,310, filed Nov. 7, 2022, 63/471,291, filed Jun. 6, 2023, and 63/522,355, filed Jun. 21, 2023, which are hereby incorporated by reference in their entirety, and to which applications we claim priority.

### FIELD OF THE INVENTION

The present invention relates generally to methods for treating cholestasis in a subject having liver disease. More specifically, the present invention relates to methods for treating Progressive Familial Intrahepatic Cholestasis (PFIC) in a subject where the method includes administering maralixibat to a subject in need thereof.

### BACKGROUND

Progressive familial intrahepatic cholestasis (PFIC) is a rare autosomal recessive liver disorder characterized by intrahepatic cholestasis due to canalicular bile transport defects. There are more than 4 subtypes of PFIC classified based on different mutations. PFIC 1, 2, and 3 result from mutations in the ATPase phospholipid transporting 8B1 (ATP8B1), ATP binding cassette subfamily B member 11 (ABCB11), and ATP binding cassette subfamily B member 4 (ABCB4) genes, respectively, and all share the main clinical manifestations of cholestasis and pruritus. PFIC 4 is based on mutations in the tight junction protein 2 gene (TJP2) and leads to failure of protein localization, disruption of tight-junction structure, and severe cholestatic liver disease. In children, PFIC represents 10%-15% of causes of cholestasis and 10%-15% of indications for liver transplantations. PFIC 2 is the most common subtype and is diagnosed in approximately 50%-60% of PFIC patients while PFIC 1 (also known as Byler's disease) and PFIC 3 account for approximately 10%-20% and 30%-40% of the PFIC population, respectively.

PFIC is associated with early mortality, morbidity, and devastating consequences on patients' quality of life. In the absence of surgery, PFIC 1 and PFIC 2 are very aggressive diseases with only 10%-15% of PFIC 1 and PFIC 2 (depending on the variant) subjects surviving through the age of 18. PFIC 2 is associated with a continuous progressive course of symptoms. While extrahepatic involvement such as pancreatitis or diarrhea can be a feature of PFIC 1, the initial presentation and evolution of the disease in PFIC 2 tends to be more severe than in PFIC 1, with persistent jaundice occurring within the first months of life and rapid progression to cirrhosis and liver failure within the first years of life. Interruption of the entero-hepatic circulation of bile acids through partial external biliary diversion (PEBD) surgery can lead to promising results with respect to pruritus, jaundice, and histology, both in patients with PFIC 1 and PFIC 2. A previous study reported a dramatic 1-year outcome in patients undergoing PEBD with 13/21 (62%) of patients normalizing serum bile acids (sBAs) and liver function; however, other groups have reported overall failure rates of PEBD of up to 30% with 30%-50% of patients requiring repeat surgery. Additionally, PEBD must be performed before liver fibrosis and cirrhosis are established for optimal benefit. For the vast majority of patients who do not undergo PEBD or do not respond, liver transplantation may be the only treatment option. Given the clinical outcomes associated with PFIC, including the profound negative impact on patients' and caregivers' quality of life, and the fact that there are no approved treatments, there is a clear unmet medical need for a novel treatment for this disease.

Maralixibat (as maralixibat chloride) is currently the only approved medication to treat pruritus in people with Alagille syndrome. It is known that maralixibat chloride inhibits apical sodium co-dependent bile acid transport (U.S. Pat. No. 5,994,391). The synthesis of maralixibat chloride is previously disclosed in U.S. patent application Pub. No. 2003/0199515A1.

### SUMMARY OF THE INVENTION

Various non-limiting aspects and embodiments of the invention are described below.

In one aspect, the present invention provides a method for treating a progressive familial intrahepatic cholestasis (PFIC) in a subject in need thereof comprising administering to the subject maralixibat, or a pharmaceutically acceptable salt thereof.

In one embodiment, the pharmaceutically acceptable salt of maralixibat is maralixibat chloride, maralixibat bromide, maralixibat acetate, or maralixibat mesylate. In one embodiment, the pharmaceutically acceptable salt of maralixibat is maralixibat chloride.

In one embodiment, the maralixibat or pharmaceutically acceptable salt thereof is administered in an amount from about 10 μg/kg/day to about 1400 μg/kg/day. In one embodiment, the maralixibat or pharmaceutically acceptable salt thereof is administered in an amount from about 300 μg/kg/day to about 1200 μg/kg/day. In one embodiment, the maralixibat or pharmaceutically acceptable salt thereof is administered in an amount from about 600 μg/kg/day to about 1200 μg/kg/day. In one embodiment, the maralixibat or pharmaceutically acceptable salt thereof is maralixibat chloride, and maralixibat chloride is administered in an amount of about 1200 μg/kg/day.

In one embodiment, the maralixibat or pharmaceutically acceptable salt thereof is administered in an amount of about 0.5 mg/day to about 100 mg/day.

In one embodiment, the PFIC is PFIC 1, PFIC 2, PFIC 3, PFIC 4, PFIC 5, or PFIC 6. In one embodiment, the PFIC is PFIC 1. In one embodiment, the PFIC is PFIC 2. In one embodiment, the PFIC 2 is non-truncated PFIC 2. In one embodiment, the PFIC 2 is truncated PFIC 2. In one embodiment, the PFIC is PFIC 3. In one embodiment, the PFIC is PFIC 4. In one embodiment, the PFIC is PFIC 5. In one embodiment, the PFIC is PFIC 6.

In one embodiment, the PFIC is heterozygous. In one embodiment, the subject has intermittent cholestasis. In one embodiment, the subject has undergone biliary diversion surgery.

In one embodiment, the subject is a pediatric subject. In one embodiment, the subject is more than 1 year old and less than 18 years old. In one embodiment, the subject is less than 12 months old.

In one embodiment, the subject has a mutation in a gene selected from the group consisting of: ATP8B1, ABCB11, ABCB4, TJP2, NR1H4, and MYO5B.

In one embodiment, the maralixibat or pharmaceutically acceptable salt thereof is administered once daily (QD). In one embodiment, the maralixibat or pharmaceutically acceptable salt thereof is administered twice daily (BID).

In one embodiment, the maralixibat or pharmaceutically acceptable salt thereof is maralixibat chloride, and maralixibat chloride is administered at 600 μg/kg/day BID for a total daily dose of 1200 μg/kg/day.

In one embodiment, the administration of the maralixibat or pharmaceutically acceptable salt thereof results in the reduction in a symptom or a change in a disease-relevant laboratory measure of PFIC that is maintained for at least 2 months. In one embodiment, the administration of the maralixibat or pharmaceutically acceptable salt thereof results in the reduction in a symptom or a change in a disease-relevant laboratory measure of PFIC that is maintained for at least 4 months. In one embodiment, the administration of the maralixibat or pharmaceutically acceptable salt thereof results in the reduction in a symptom or a change in a disease-relevant laboratory measure of PFIC that is maintained for at least 6 months. In one embodiment, the administration of the maralixibat or pharmaceutically acceptable salt thereof results in the reduction in a symptom or a change in a disease-relevant laboratory measure of PFIC that is maintained for at least 1 year.

In one embodiment, the reduction in the symptom or the change in the disease-relevant laboratory measure is determined relative to a baseline level.

In one embodiment, the reduction in the symptom or change in the disease-relevant laboratory measure comprises a reduction in sBA concentration, a reduction in pruritus, a reduction in total bilirubin, a reduction in direct bilirubin, an improvement in growth, or a combination thereof.

In one embodiment, the administration of the maralixibat reduces intensity of pruritus. In one embodiment, the reduction of intensity of pruritus is a reduction of an ItchRO(Obs) score, of a CSS score, or a combination thereof.

In one embodiment, the administration of the maralixibat or pharmaceutically acceptable salt thereof results in a reduction of an ItchRO(Obs) score of the subject by at least 1.0 points relative to baseline. In one embodiment, the administration of the maralixibat or pharmaceutically acceptable salt thereof results in a reduction of an ItchRO(Obs) score of the subject by at least 1.2 points relative to baseline. In one embodiment, the administration of the maralixibat or pharmaceutically acceptable salt thereof results in a reduction of an ItchRO(Obs) score of the subject by at least 1.4 points relative to baseline. In one embodiment, the administration of the maralixibat or pharmaceutically acceptable salt thereof results in a reduction of an ItchRO(Obs) score of the subject by at least 1.6 points relative to baseline.

In one embodiment, the administration of the maralixibat or pharmaceutically acceptable salt thereof results in a reduction of a CSS score of the subject by at least 1.0 points relative to baseline. In one embodiment, the administration of the maralixibat or pharmaceutically acceptable salt thereof results in a reduction of a CSS score of the subject by at least 1.2 points relative to baseline. In one embodiment, the administration of the maralixibat or pharmaceutically acceptable salt thereof results in a reduction of a CSS score of the subject by at least 1.4 points relative to baseline. In one embodiment, the administration of the maralixibat or pharmaceutically acceptable salt thereof results in a reduction of a CSS score of the subject by at least 1.6 points relative to baseline.

In one embodiment, the administration of the maralixibat or pharmaceutically acceptable salt thereof results in a reduction in sBA concentration in the subject by at least 50 μmol/L relative to baseline. In one embodiment, the administration of the maralixibat or pharmaceutically acceptable salt thereof results in a reduction in sBA concentration in the subject by at least 100 μmol/L relative to baseline.

In one embodiment, the administration of the maralixibat or pharmaceutically acceptable salt thereof results in a reduction in total bilirubin. In one embodiment, the administration of the maralixibat or pharmaceutically acceptable salt thereof results in a reduction in total bilirubin of at least 0.2 mg/dL relative to baseline. In one embodiment, the administration of the maralixibat or pharmaceutically acceptable salt thereof results in a reduction in total bilirubin of at least 0.5 mg/dL relative to baseline. In one embodiment, the administration of the maralixibat or pharmaceutically acceptable salt thereof results in a reduction in total bilirubin of at least 1.0 mg/dL relative to baseline.

In one embodiment, the administration of the maralixibat or pharmaceutically acceptable salt thereof results in a reduction in direct bilirubin. In one embodiment, the administration of the maralixibat or pharmaceutically acceptable salt thereof results in a reduction in direct bilirubin of at least 0.2 mg/dL relative to baseline. In one embodiment, the administration of the maralixibat or pharmaceutically acceptable salt thereof results in a reduction in direct bilirubin of at least 0.5 mg/dL relative to baseline. In one embodiment, the administration of the maralixibat or pharmaceutically acceptable salt thereof results in a reduction in direct bilirubin of at least 1.0 mg/dL relative to baseline.

In one embodiment, the administration of the maralixibat or pharmaceutically acceptable salt thereof results in an improvement in a height Z-score or a weight Z-score, or both in the subject relative to baseline. In one embodiment, the administration of the maralixibat or pharmaceutically acceptable salt thereof results in an increase in the weight Z-score of at least 0.2 relative to baseline.

In one embodiment, the method further comprises administering a lipid soluble vitamin (LSV) in subjects with LSV deficiency. In one embodiment, the LSV is selected from the group consisting of Vitamin A, Vitamin D and Vitamin E.

In one embodiment, the maralixibat or pharmaceutically acceptable salt thereof is administered before a meal. In one embodiment, the maralixibat or pharmaceutically acceptable salt thereof is administered about 30 minutes before a meal. In one embodiment, wherein the maralixibat or pharmaceutically acceptable salt thereof is administered BID, about 30 minutes before the morning meal and about 30 minutes before the evening meal.

In one embodiment, the maralixibat is administered in the form of a pharmaceutical composition comprising maralixibat, or a pharmaceutically acceptable salt thereof, an antioxidant, and a preservative. In one embodiment, the pharmaceutical composition is a liquid composition for oral administration. In one embodiment, the liquid composition is an aqueous solution.

In one embodiment, the maralixibat is present in an amount of about 2 mg/mL to about 100 mg/mL of the composition. In one embodiment, the maralixibat is present in an amount of about 5 mg/mL to about 50 mg/mL of the composition. In one embodiment, the maralixibat is present in an amount of about 8 mg/mL to about 20 mg/mL of the composition. In one embodiment, the maralixibat is present in an amount of about 9.5 mg/mL to about 10 mg/mL of the composition.

In one embodiment, the preservative is an antimicrobial preservative. In one embodiment, the antimicrobial preservative is selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof. In one embodiment, the preservative is propylene glycol. In one embodiment, the preservative is present in an amount from about 30% w/w to about 40% w/w of the composition. In one embodiment, the preservative is present in an amount from about 300 mg/mL to about 400 mg/mL of the composition.

In one embodiment, the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

In one embodiment, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid). In one embodiment, the antioxidant is EDTA. In one embodiment, the antioxidant is present in an amount of about 0.01% w/w to about 0.5% w/w of the composition.

In one embodiment, the pharmaceutical composition further comprises a sweetener, a taste-masking ingredient, or a combination thereof.

In one embodiment, the pharmaceutical composition comprises:
a. from about 8 mg/mL to about 20 mg/mL of maralixibat;
b. from about 330 mg/mL to about 380 mg/mL of propylene glycol;
c. about 1 mg/mL of disodium EDTA;
d. a sweetener, a taste-masking ingredient, or a combination thereof, and
e. water.

These and other aspects of the present invention will become apparent to those skilled in the art after a reading of the following detailed description of the invention, including the appended claims.

### BRIEF DESCRIPTION OF THE DRAWINGS

FIG. 1 provides a schematic diagram summarizing physiological effects of maralixibat administration in a patient. IBAT, ileal bile acid transporter.

FIG. 2 shows a schematic providing an overview of a dosing regimen used in a phase 3 open-label extension clinical study of maralixibat in subjects with PFIC. The clinical study investigated long-term exposure to maralixibat.

FIG. 3 shows a flow diagram representing the protocol recommendations for treatment of and discontinuation due to worsening of LSV deficiency.

FIG. 4A outlines the MARCH-PFIC Phase 3 Study Design. FIG. 4B shows Efficacy Endpoints. *Maralixibat 570 μg/kg is equivalent to 600 μg/kg maralixibat chloride. Abbreviations: AE=adverse event; BSEP=bile salt export pump; BL=Baseline; ItchRO(Obs)=Itch-Reported Outcome (Observer); MMRM=mixed model repeated measures; R=randomised.

FIG. 5 describes the 93 MARCH-PFIC study participants.

FIGS. 6A and 6B show BSEP deficiency cohort (FIG. 6A) and All PFIC cohort (FIG. 6B): Mean change from baseline in pruritus severity score (ItchRO[Obs]). Abbreviations: LS=Least squares; SE=standard error of the mean; CFB=change from baseline; CI=confidence interval.

FIGS. 7A and 7B show the mean change from baseline in evening (FIG. 7A) and maximum daily (FIG. 7B) pruritus severity score (ItchRO[Obs]) for the BSEP deficiency cohort.

FIGS. 8A and 8B show the mean change from baseline in evening (FIG. 8A) and maximum daily (FIG. 8B) pruritus severity score (ItchRO[Obs]) for the All PFIC cohort.

FIGS. 9A and 9B show the mean change from baseline in pruritus severity score (ItchRO[Obs]) for the FIC1 (FIG. 9A) and MDR3 (FIG. 9B) deficiencies cohorts.

FIGS. 10A and 10B show change from baseline in pruritus severity score (ItchRO[Obs]) over time for the BSEP deficiency (aka Primary) cohort (FIG. 10A), PFIC cohort (FIG. 10B), and all study participants (FIG. 10C).

FIGS. 11A and 11B show the Key Secondary Efficacy Endpoint in BSEP deficiency cohort (FIG. 11A) and All PFIC cohort (FIG. 11B): Mean change from baseline in sBA levels. Abbreviations: LS=Least squares; SE=standard error of the mean; CFB=change from baseline; CI=confidence interval.

FIGS. 12A and 12B show the Mean change from baseline in sBA levels in FIC1 cohort (FIG. 12A) and MDR3 cohort (FIG. 12B).

FIGS. 13A and 13B show percentage of pruritus (FIG. 13A) and sBA (FIG. 13B) response as per MARCH SAP for the BSEP deficiency cohort.

FIGS. 14A and 14B show percentage of pruritus (FIG. 14A) and sBA (FIG. 14B) response as per MARCH SAP for the All PFIC cohort.

FIGS. 15A and 15B show change from baseline in sBA over time for the BSEP deficiency (aka Primary) cohort (FIG. 15A), PFIC cohort (FIG. 15B) and all study participants (FIG. 15C).

FIGS. 16A and 16B show Pruritus response proportion of assessments ≤1 in BSEP Deficiency and PFIC Cohorts. The proportion of BSEP deficiency cohort responders is shown in FIG. 16A; the proportion of all PFIC cohort responders is shown in FIG. 16B.

FIGS. 17A and 17B show Pruritus response proportion of assessments ≤1 or decrease ≥1 in B SEP Deficiency and PFIC Cohorts. The proportion of B SEP deficiency cohort responders is shown in FIG. 17A; the proportion of all PFIC cohort responders is shown in FIG. 17B.

FIGS. 18A and 18B show clinician scratch score over time for the BSEP deficiency (aka Primary) cohort (FIG. 18A) and PFIC cohort (FIG. 18B).

FIGS. 19A and 19B show mean change from baseline in clinician scratch scale score for the BSEP deficiency cohort (FIG. 19A) and PFIC cohort (FIG. 19B).

FIGS. 20A and 20B show mean change from baseline in total bilirubin (mg/dL) for the B SEP deficiency cohort (FIG. 20A) and PFIC cohort (FIG. 20B).

FIGS. 21A and 21B show change from baseline in total bilirubin (mg/dL) over time for the BSEP deficiency (aka Primary) cohort (FIG. 21A) and PFIC cohort (FIG. 21B).

FIGS. 22A and 22B show mean change from baseline in direct bilirubin (mg/dL) for the B SEP deficiency cohort (FIG. 22A) and PFIC cohort (FIG. 22B).

FIGS. 23A and 23B show change from baseline in direct bilirubin (mg/dL) over time for the BSEP deficiency (aka Primary) cohort (FIG. 23A) and PFIC cohort (FIG. 23B).

FIGS. 24A-24C show change from baseline in height Z-score over time for the BSEP deficiency (aka Primary) cohort (FIG. 24A) and PFIC cohort (FIGS. 24B and 24C).

FIGS. 25A-25C show change from baseline in weight Z-score over time for the BSEP deficiency (aka Primary) cohort (FIG. 25A) and PFIC cohort (FIGS. 25B and 25C).

FIGS. 26A and 26B show change from baseline ALT in All-PFIC Cohort.

FIG. 27A shows a schematic providing an overview of the Method. *ItchRO(Obs) score ≥1.5; †Maralixibat 570 μg/kg is equivalent to 600 μg/kg maralixibat chloride. FIG. 27B shows that patients on maralixibat had significantly more days with minimal to no itch than patients on placebo. Error bars represent SE. Percentage values represent the proportion of assessments from Baseline to Week 26. *Delta with 95% Cl. CI, confidence interval. SE is a standard error. FIG. 27C shows that patients on maralixibat had significant reduction in pruritus compared to placebo irrespective of when and how it was measured. FIG. 27D shows significant improvements in change from Baseline sleep with maralixibat vs placebo, measured via the EDQ(Obs). *Delta with 95% CI. FIG. 27E shows that change in pruritus was strongly correlated with change in sleep. Abbreviations: BL=Baseline; CSS=Clinician Scratch Scale; EDQ(Obs)=Exploratory Diary Questionnaire (Observer); ItchRO(Obs)=Itch-Reported Outcome (Observer); R=randomized.

FIG. 28 shows incidence of gastrointestinal events.

FIG. 29A shows a schematic providing a study design. FIGS. 29B and 29C show that significant improvements in pruritus severity (FIG. 29B) and serum bile acids (sBA) levels (FIG. 29C) were sustained in the MRX-MRX group. FIGS. 29D and 29E show that newly gained significant reductions in pruritus severity (FIG. 29D) and sBA levels (FIG. 29E) were observed in the PBO-MRX group.

FIG. 30 shows a schematic providing a study design, ^{a}All doses presented as MRX-free base. All patients received the daily dose appropriate for their weight, with an adult maximum dose of 28.5 mg/d. ^{b}Before age 16 years is defined as the last data point prior to turning 16 years of age. After age 16 years is defined as the first data point after turning 16 years of age.

FIGS. 31A and 31B show a change in ltchRO(Obs) (FIG. 31A) and sBA (FIG. 31B) for participants who initiated MRX treatment at <16 years of age (n=11). Of 11 participants, 9 had ItchRO(Obs) scores available for all time points and were included in the analysis. ^{b}ItchRO(Obs) is a 0-4 scale with >1-point reduction considered clinically meaningful. Mean ItchRO(Obs) was rounded to 1 decimal place. Error bars represent SE. Significance was determined using student t test. ^{c}Mean sBA was rounded to the nearest whole number. Error bars represent SE. ^{d}Average of last 2 records prior to age 16 years. ^{e}Average of first 2 records after age 16 years. ^{f}Average of final 2 records.

FIGS. 32A and 32B show a change in ltchRO(Obs) (FIG. 32A) and sBA (FIG. 32B) for participants who Initiated MRX treatment at ≥16 years of age (n=3). ^{a}ItchRO(Obs) is 0-4 scale with ≥1-point reduction considered clinically meaningful. ^{b}Average of final two records.

FIGS. 33A and 33B show change is weekly ItchRO(Obs) score in FIC1, MDR3, TJP2, and MYO5B.

FIGS. 34A and 34B show change from baseline in Serum Bile Acid in FIC1, MDR3, TJP2, and MYO5B.

FIGS. 35A-35H show change in weekly ltchRO(Obs) score and serum bile acids (sBA) in full-study cohort (FIGS. 35A and 35B), no-variant-found cohort (FIGS. 35C and 35D), FIC1 cohort (FIGS. 35E and 35F), and MDR3 (FIGS. 35G and 35H).

FIGS. 36A and 36B show change from Baseline in total (FIG. 36A) and direct bilirubin (FIG. 36B) in All-PFIC cohort.

FIGS. 37A and 37B show change from baseline in patients with abnormal total (FIG. 37A) and direct bilirubin (FIG. 37B) at Baseline.

FIGS. 38A and 38B show total bilirubin normalization.

FIG. 39 shows direct bilirubin normalization.

FIG. 40 shows relationship between total bilirubin normalisation and changes in serum bile acids.

FIGS. 41A-41D show patient-level changes in morning pruritus severity over time as measured by Itch-Reported Outcome (Observer). Change from Baseline in morning pruritus severity over time (Baseline to Week 26) for individual participants in the BSEP cohort (FIG. 41A); participants with FIC1, MDR3, TJP2, and MYO5B deficiencies (FIG. 41B); participants with no identified pathogenic variants (FIG. 41C); and participants with t-B SEP (BSEP3) deficiency (FIG. 41D) are shown (blue=maralixibat blue and red=placebo groups). BSEP denotes bile salt export pump, FIC1 familial intrahepatic cholestasis-associated protein 1, MDR3 multidrug resistance protein 3, MYO5B myosin VB, t truncated, and TJP2 tight junction protein 2.

FIGS. 42A-42D show patient-level changes in BA levels over time. Change from baseline in sBA levels (Baseline to Week 26) for participants in the BSEP cohort (FIG. 42A); participants with FIC1, MDR3, TJP2 and MYO5B participants (FIG. 42B); participants with no pathogenic variants found (FIG. 42C); and truncated B SEP (BSEP3; FIG. 42D) are shown (blue=maralixibat and red=placebo groups). BSEP denotes bile salt export pump, FIC1 familial intrahepatic cholestasis-associated protein 1, MDR3 multidrug resistance protein 3, MYO5B myosin VB, and t truncated. *Patient had concomitant reductions in total bilirubin (3.85 mg/dL at Baseline to 2.6 mg/dL at Week 26) and ALT (320 U/L at Baseline to 183 U/L at Week 26).

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

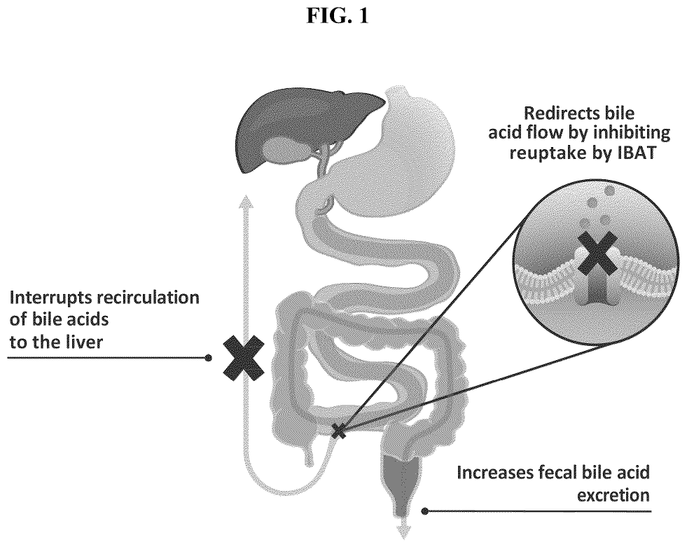

Maralixibat is an inhibitor of IBAT, a transmembrane protein present in the terminal ileum and is localized on the luminal surface of ileal enterocytes. IBAT mediates the uptake of conjugated bile acids across the brush border membrane of the enterocyte. Ninety-five percent of bile acids that enter the gut lumen are recycled to the gall bladder where they are stored for future release to the duodenum. Additional proteins and transporters carry bile acids through the enterocyte and across the basolateral membrane into the blood stream, where they are circulated to the liver via the portal vein and then re-secreted into the intestine, known as enterohepatic circulation (FIG. 1). Maralixibat-mediated blockade of intestinal reabsorption of bile acids by IBAT interrupts the enterohepatic circulation, thereby increasing fBA excretion and lowering sBA levels.

“Maralixibat” refers to 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium, which is the free form of maralixibat chloride. The structure of maralixibat is represented below:

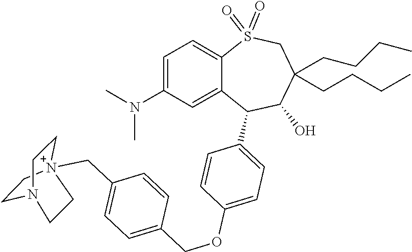 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

“Maralixibat chloride” (also known as LUM-001, SHP625, or lopixibat chloride) refers to 1-(4-((4-((4R, 5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium chloride. The structure of maralixibat chloride is represented below:

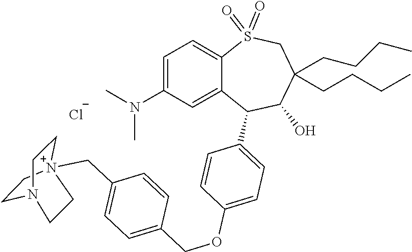 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

The IBAT is ideally suited for pharmacological modulation of bile acid transport by a compound that can be restricted to the lumen of the gut, one that is not required to have systemic exposure for activity. Maralixibat was designed to be minimally absorbed due to its large molecular weight (˜710 Da) and the presence of a positively charged quaternary nitrogen atom, therefore maximizing the local exposure of the molecule to its target and minimizing unnecessary systemic exposure.

Because reductions in sBA concentrations after surgical interruption of the enterohepatic circulation have been shown to be associated with improvements in cholestasis and clinical outcomes in several pediatric cholestatic liver diseases pharmacological interruption of the enterohepatic circulation by IBAT inhibition represents a potential nonsurgical and easily reversible alternative to achieve a similar reduction in sBA, and thus has the potential to improve outcomes in diseases such as PFIC, ALGS, and biliary atresia.

Bile acids/salts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced, and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectoral transfer of bile acids/salts into the portal circulation. Completion of the enterohepatic circulation occurs at the basolateral surface of the hepatocyte by a transport process that is primarily mediated by a sodium-dependent bile acid transporter. Intestinal bile acid transport plays a key role in the enterohepatic circulation of bile salts. Molecular analysis of this process has recently led to important advances in understanding of the biology, physiology, and pathophysiology of intestinal bile acid transport.

Within the intestinal lumen, bile acid concentrations vary, with the bulk of the reuptake occurring in the distal intestine. Described herein are certain compositions and methods that control bile acid concentrations in the intestinal lumen, thereby controlling the hepatocellular damage caused by bile acid accumulation in the liver.

### General Definitions

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “baseline” or “pre-administration baseline,” as used herein, refers to information gathered at the beginning of a study or an initial known value which is used for comparison with later data. A baseline is an initial measurement of a measurable condition that is taken at an early time point and used for comparison over time to look for changes in the measurable condition. For example, serum bile acid concentration in a patient before administration of a drug (baseline) and after administration of the drug. Baseline is an observation or value that represents the normal or beginning level of a measurable quality, used for comparison with values representing response to intervention or an environmental stimulus. The baseline is time “zero”, before participants in a study receive an experimental agent or intervention, or negative control. For example, “baseline” may refer in some instances 1) to the state of a measurable quantity just prior to the initiation of a clinical study or 2) the state of a measurable quantity just prior to altering a dosage level or composition administered to a patient from a first dosage level or composition to a second dosage level or composition.

The terms “level” and “concentration,” as used herein, are used interchangeably. For example, “high serum levels of bilirubin” may alternatively be phrased “high serum concentrations of bilirubin.”

The terms “normalized” or “normal range,” as used herein, indicates age-specific values that are within a range corresponding to a healthy individual (i.e., normal or normalized values). For example, the phrase “serum bilirubin concentrations were normalized within three weeks” means that serum bilirubin concentrations fell within a range known in the art to correspond to that of a healthy individual (i.e., within a normal and not e.g. an elevated range) within three weeks. In various embodiments, a normalized serum bilirubin concentration is from about 0.1 mg/dL to about 1.2 mg/dL. In various embodiments, a normalized serum bile acid concentration is from about 0 μmon to about 25 μmon.

The terms “ITCHRO(OBS)” and “ITCHRO” (alternatively, “ItchRO(Pt)”) as used herein, are used interchangeably with the qualification that the ITCHRO(OBS) scale is used by a caregiver to measure severity of pruritus in all patients and the ITCHRO scale is used to measure severity of pruritus in adults of at least 18 years of age. Therefore, where ITCHRO(OBS) scale is mentioned with regard to an adult patient, the ITCHRO scale is the scale being indicated. Similarly, whenever the ITCHRO scale is mentioned with regard to a pediatric patient, the ITCHRO(OBS) scale is usually the scale being indicated. Some children who were at least 9 years old reported their own scores as ITCHRO(Pt) scores). For the ALGS and PFIC studies, ITCHRO(Pt) was done by patients who were at least 9 years old and ITCHRO(OBS) was used by patients under 9 years of age. The ITCHRO(OBS) scale ranges from 0 to 4, the ITCHRO(Pt) scale ranges from 0 to 4, and the ITCHRO scale ranges from 0 to 10.

The terms “EDQ(Obs)”, “EDQ(Pt)”, and “EDQ” as used herein, refer to Exploratory Diary Questionnaire that are used to assess pruritus in children under the age of 18 or adults of at least 18 years of age. EDQ is a caregiver/patient reported outcome measure administered as a twice daily electronic diary. EDQ contained a question about sleep related to pruritus: “because of itch, my child had trouble staying asleep. 1—Never, 2—Rarely, 3—ometimes, 4—Often, or 5—Almost Always.” Caregivers for all subjects aged <9 years completed the Observer instrument: EDQ(Obs). Subjects ≥9 years of age completed the patient instrument: EDQ(Pt). EDQ includes questions on sleep disturbances related to pruritus.

The term “bile acid” or “bile acids,” as used herein, includes steroid acids (and/or the carboxylate anion thereof), and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate, chenodeoxycholic acid, ursodeoxycholic acid, ursodiol, a tauroursodeoxycholic acid, a glycoursodeoxycholic acid, a 7-B-methyl cholic acid, a methyl lithocholic acid, chenodeoxycholate, lithocholic acid, lithocolate, and the like. Taurocholic acid and/or taurocholate are referred to herein as TCA. Any reference to a bile acid used herein includes reference to a bile acid, one and only one bile acid, one or more bile acids, or to at least one bile acid. Therefore, the terms “bile acid,” “bile salt,” “bile acid/salt,” “bile acids,” “bile salts,” and “bile acids/salts” are, unless otherwise indicated, utilized interchangeably herein. Any reference to a bile acid used herein includes reference to a bile acid or a salt thereof. Furthermore, pharmaceutically acceptable bile acid esters are optionally utilized as the “bile acids” described herein, e.g., bile acids/salts conjugated to an amino acid (e.g., glycine or taurine). Other bile acid esters include, e.g., substituted or unsubstituted alkyl ester, substituted or unsubstituted heteroalkyl esters, substituted or unsubstituted aryl esters, substituted or unsubstituted heteroaryl esters, or the like. For example, the term “bile acid” includes cholic acid conjugated with either glycine or taurine: glycocholate and taurocholate, respectively (and salts thereof). Any reference to a bile acid used herein includes reference to an identical compound naturally or synthetically prepared. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted.

The term “subject”, “patient”, “participant”, or “individual” are used interchangeably herein and refer to mammals and non-mammals, e.g., suffering from a disorder described herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “about,” as used herein, includes any value that is within 10% of the described value.

The term “composition,” as used herein includes the disclosure of both a composition and a composition administered in a method as described herein. Furthermore, in some embodiments, the composition of the present invention is or comprises a “formulation,” an oral dosage form or a rectal dosage form as described herein.

The terms “treat,” “treating” or “treatment,” and other grammatical equivalents as used herein, include alleviating, inhibiting or reducing symptoms, reducing or inhibiting severity of, reducing incidence of, reducing or inhibiting recurrence of, delaying onset of, delaying recurrence of, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “effective amount” or “therapeutically effective amount” as used herein, refer to a sufficient amount of at least one agent (e.g., a therapeutically active agent) being administered which achieve a desired result in a subject or individual, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study. In some embodiments, a “therapeutically effective amount,” or an “effective amount” of an ASBTI refers to a sufficient amount of an ASBTI to treat cholestasis or a cholestatic liver disease in a subject or individual.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa, all of which are incorporated herein by reference in their entirety for all purposes. In certain embodiments, the agents and compositions described herein are administered orally.

The term “ASBT inhibitor” refers to a compound that inhibits apical sodium-dependent bile transport or any recuperative bile salt transport. The term “IBAT inhibitor” refers to a compound that inhibits ileal bile acid transport or any recuperative bile salt transport. The term Apical Sodium-dependent Bile Transporter (ASBT) is used interchangeably with the term Ileal Bile Acid Transporter (IBAT). The term “ASBT inhibitor” is used interchangeably with the term “IBAT inhibitor.”

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, subsalicylate, maleate, laurate, malate, fumarate, succinate, tartrate, amsonate, pamoate, p-toluenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

### Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acids/salts are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acids/salts are derivatives of cholesterol. Cholesterol, ingested as part of the diet or derived from hepatic synthesis, are converted into bile acids/salts in the hepatocyte. Examples of such bile acids/salts include cholic and chenodeoxycholic acids, which are then conjugated to an amino acid (such as glycine or taurine) to yield the conjugated form that is actively secreted into cannaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acids/salts and lipids. Hepatic synthesis of bile acids/salts accounts for the majority of cholesterol breakdown in the body. In humans, roughly 500 mg of cholesterol are converted to bile acids/salts and eliminated in bile every day. Therefore, secretion into bile is a major route for elimination of cholesterol. Large amounts of bile acids/salts are secreted into the intestine every day, but only relatively small quantities are lost from the body. This is because approximately 95% of the bile acids/salts delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acids/salts very efficiently from sinusoidal blood, and little escapes the healthy liver into systemic circulation. Bile acids/salts are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Recycling thus enables 20-30 g of bile salts to be secreted into the small intestine each day.

Bile acids/salts are amphipathic, with the cholesterol-derived portion containing both hydrophobic (lipid soluble) and polar (hydrophilic) moieties while the amino acid conjugate is generally polar and hydrophilic. This amphipathic nature enables bile acids/salts to carry out two important functions: emulsification of lipid aggregates and solubilization and transport of lipids in an aqueous environment. Bile acids/salts have detergent action on particles of dietary fat which causes fat globules to break down or to be emulsified. Emulsification is important since it greatly increases the surface area of fat available for digestion by lipases which cannot access the inside of lipid droplets. Furthermore, bile acids/salts are lipid carriers and are able to solubilize many lipids by forming micelles and are critical for transport and absorption of the fat-soluble vitamins.

The term “non-systemic” or “minimally absorbed,” as used herein, refers to low systemic bioavailability and/or absorption of an administered compound. In some embodiments a non-systemic compound is a compound that is substantially not absorbed systemically. In some embodiments, ASBTI compositions described herein deliver the ASBTI to the distal ileum, colon, and/or rectum and not systemically (e.g., a substantial portion of the ASBTI is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <10% of the administered dose. In some embodiments, the systemic absorption of a non-systemic compound is <15% of the administered dose. In some embodiments, the systemic absorption of a non-systemic compound is <25% of the administered dose. In an alternative approach, a non-systemic ASBTI (e.g., maralixibat) is a compound that has lower systemic bioavailability relative to the systemic bioavailability of a systemic ASBTI. In some embodiments, the bioavailability of a non-systemic ASBTI described herein (e.g., maralixibat) is <30%, <40%, <50%, <60%, or <70% of the bioavailability of a systemic ASBTI.

In another alternative approach, compositions described herein are formulated to deliver <10% of the administered dose of the ASBTI systemically. In some embodiments, the compositions described herein are formulated to deliver <20% of the administered dose of the ASBTI systemically. In some embodiments, the compositions described herein are formulated to deliver <30% of the administered dose of the ASBTI systemically. In some embodiments, the compositions described herein are formulated to deliver <40% of the administered dose of the ASBTI systemically. In some embodiments, the compositions described herein are formulated to deliver <50% of the administered dose of the ASBTI systemically. In some embodiments, the compositions described herein are formulated to deliver <60% of the administered dose of the ASBTI systemically. In some embodiments, the compositions described herein are formulated to deliver <70% of the administered dose of the ASBTI systemically. In some embodiments, systemic absorption is determined in any suitable manner, including the total circulating amount, the amount cleared after administration, or the like.

### Classes of Cholestasis and Cholestatic Liver Disease

As used herein, “cholestasis” means the disease or symptoms comprising impairment of bile formation and/or bile flow. As used herein, “cholestatic liver disease” means a liver disease associated with cholestasis. Cholestatic liver diseases are often associated with jaundice, fatigue, and pruritus. Biomarkers of cholestatic liver disease include elevated serum bile acid concentrations, elevated serum alkaline phosphatase (AP), elevated gamma-glutamyltranspeptidease (gammaGT), elevated conjugated hyperbilirubinemia, and elevated serum cholesterol.

Cholestatic liver disease can be sorted clinicopathologically between two principal categories of obstructive, often extrahepatic, cholestasis, and nonobstructive, or intrahepatic, cholestasis. In the former, cholestasis results when bile flow is mechanically blocked, as by gallstones or tumor, or as in extrahepatic biliary atresia.

The latter group who has nonobstructive intrahepatic cholestasis in turn fall into two principal subgroups. In the first subgroup, cholestasis results when processes of bile secretion and modification, or of synthesis of constituents of bile, are caught up secondarily in hepatocellular injury so severe that nonspecific impairment of many functions can be expected, including those subserving bile formation. In the second subgroup, no presumed cause of hepatocellular injury can be identified. Cholestasis in such patients appears to result when one of the steps in bile secretion or modification, or of synthesis of constituents of bile, is constitutively damaged. Such cholestasis is considered primary.

### Progressive Familial Intrahepatic Cholestasis (PFIC)

PFIC is a rare genetic disorder that causes progressive liver disease typically leading to liver failure. In people with PFIC, liver cells are less able to secrete bile. The resulting buildup of bile causes liver disease in affected individuals. Signs and symptoms of PFIC typically begin in infancy. Patients experience severe itching, jaundice, failure to grow at the expected rate (failure to thrive), and an increasing inability of the liver to function (liver failure). The disease is estimated to affect one in every 50,000 to 100,000 births in the United States and Europe. Six types of PFIC have been genetically identified, all of which are similarly characterized by impaired bile flow and progressive liver disease.

PFIC types include familial intrahepatic cholestasis-associated protein 1 (FIC1) deficiency (PFIC1), bile salt export pump (BSEP) deficiency (PFIC2), multidrug resistant 3 protein (MDR3) deficiency (PFIC3), tight junction protein 2 (TJP2) deficiency (PFIC4), Farnesoid X receptor (FXR) deficiency (PFIC5), and myosin VB (MYO5B) deficiency (PFIC6), discussed in more detail below.

### PFIC 1

PFIC 1 (also known as, Byler disease or FIC1 deficiency) is associated with mutations in the ATP8B1 gene (also designated as FIC1). This gene, which encodes a P-type ATPase, is located on human chromosome 18 and is also mutated in the milder phenotype, benign recurrent intrahepatic cholestasis (BRIC) type 1 and in Greenland familial cholestasis. FIC1 protein is located on the canalicular membrane of the hepatocyte but within the liver it is mainly expressed in cholangiocytes. P-type ATPase appears to be an aminophospholipid transporter responsible for maintaining the enrichment of phosphatidylserine and phophatidylethanolamme on the inner leaflet of the plasma membrane in comparison of the outer leaflet. In some instances, the PFIC 1 mutation is homozygous. In other instances, the PFIC 1 mutation is heterozygous. The asymmetric distribution of lipids in the membrane bilayer plays a protective role against high bile salt concentrations in the canalicular lumen. The abnormal protein function may indirectly disturb the biliary secretion of bile acids. The anomalous secretion of bile acids/salts leads to hepatocyte bile acid overload.

PFIC 1 typically presents in infants (e.g., age 6-18 months). The infants may show signs of pruritus, jaundice, abdominal distension, diarrhea, malnutrition, and shortened stature. Biochemically, individuals with PFIC 1 have elevated serum transaminases, elevated bilirubin, elevated serum bile acid levels, and low levels of gammaGT. The individual may also have liver fibrosis. Individuals with PFIC 1 typically do not have bile duct proliferation. Most individuals with PFIC 1 will develop end-stage liver disease by 10 years of age. No medical treatments have proven beneficial for the long-term treatment of PFIC 1. In order to reduce extrahepatic symptoms (e.g., malnutrition and failure to thrive), children are often administered medium chain triglycerides and fat-soluble vitamins. Ursodiol has not been demonstrated as effective in individuals with PFIC 1.

### PFIC 2

PFIC 2 (also known as, Byler Syndrome or BSEP deficiency) is associated with mutations in the ABCB11 gene (also designated BSEP). The ABCB11 gene encodes the ATP-dependent canalicular bile salt export pump (BSEP) of human liver and is located on human chromosome 2. BSEP protein, expressed at the hepatocyte canalicular membrane, is the major exporter of primary bile acids/salts against extreme concentration gradients. Mutations in this protein responsible for the decreased biliary bile salt secretion described in affected patients, leading to decreased bile flow and accumulation of bile salts inside the hepatocyte with ongoing severe hepatocellular damage. In some instances, the PFIC 2 mutation is homozygous. In other instances, the PFIC 2 mutation is heterozygous.

BSEP deficiency has previously been categorized into 3 subtypes, designated BSEP1, BSEP2, and BSEP3, based on the type of mutation and severity of the resulting deficiency. The BSEP1 genotype represents patients with a least one D482G or E297G mutation. BSEP1 is considered to be the least severe genotype, as the responsible mutations still allow for partial functioning of the BSEP protein. The BSEP2 genotype represents patients with at least one missense mutation that is not a D482G or E297G mutation. The BSEP3 genotype is the most severe and represents patients with mutations that are either known or predicted to lead to a non-functional BSEP protein or to absent BSEP expression. BSEP3 is associated with relatively high incidences of Hepatocellular Carcinoma (HCC). The severity of BSEP deficiency has also been shown to strongly predict long-term native liver survival.

PFIC 2 typically presents in infants (e.g., age 6-18 months). The infants may show signs of pruritus. Biochemically, individuals with PFIC 2 have elevated serum transaminases, elevated bilirubin, elevated serum bile acid levels, and low levels of gammaGT. The individual may also have portal inflammation and giant cell hepatitis. Further, individuals often develop hepatocellular carcinoma. No medical treatments have proven beneficial for the long-term treatment of PFIC 2. In order to reduce extrahepatic symptoms (e.g., malnutrition and failure to thrive), children are often administered medium chain triglycerides and fat-soluble vitamins. The PFIC 2 patient population accounts for approximately 60% of the PFIC population.

### PFIC 3

PFIC 3 (also known as MDR3 deficiency) is caused by a genetic defect in the ABCB4 gene (also designated MDR3) located on chromosome 7. Class III Multidrug Resistance (MDR3) P-glycoprotein (P-gp), is a phospholipid translocator involved in biliary phospholipid (phosphatidylcholine) excretion in the canalicular membrane of the hepatocyte. PFIC 3 results from the toxicity of bile in which detergent bile salts are not inactivated by phospholipids, leading to bile canaliculi and biliary epithelium injuries.

PFIC 3 also presents in early childhood. As opposed to PFIC 1 and PFIC 2, individuals have elevated gammaGT levels. Individuals also have portal inflammation, fibrosis, cirrhosis, and massive bile duct proliferation. Individuals may also develop intrahepatic gallstone disease. Ursodiol has been effective in treating or ameliorating PFIC 3.

### PFIC 4

PFIC 4 (also known as TJP2 deficiency) is caused by a genetic defect in the TJP2 gene (also designated zona occludens 2) located on chromosome 9. In the liver, Tight Junction Protein 2 (TJP2) is involved in forming tight junctions by interacting with transmembrane tight junction proteins and the actin cytoskeleton. Tight junctions are essential in the liver because they help prevent the leakage of biliary components into the liver parenchyma. Normally, these proteins are localized to the canalicular membrane, but in TJP2 mutation, they fail to localize, especially in the parenchyma of the hepatic lobule. These compromised tight junctions then allow cytotoxic bile salts to leak into the paracellular space, causing damage to the surrounding hepatocytes and cholangiocytes

PFIC 4 presents with severe cholestasis and low gammaGT levels. Patients lack mutations in ATP8B1 and ABCB11 genes, excluding the diagnoses of PFIC 1 and PFIC 2. Extrahepatic symptoms have been reported in some patients, mainly in the neurological and respiratory systems. There have been several reports of HCC occurrence in patients suffering with TJP2 deficiency.

### PFIC 5

PFIC 5 (also known as FXR deficiency) is caused by a genetic defect in the NR1H4 gene (also designated FXR) located on chromosome 12. Farnesoid X receptor (FXR) is a nuclear receptor activated by bile acids and directly involved in the expression of both BSEP and MDR3, proteins affected in PFIC 2 and PFIC 3 respectively. FXR is activated by elevated bile acid levels in the ileum, inducing expression of fibroblast growth factor 19 (FGF19). In the liver, FGF19 binds to the fibroblast growth factor receptor 4/β-Klotho complex, which in turn represses cytochrome P450 7A1 (CYP7A1). Repression of this enzyme reduces de novo bile acid synthesis.

PFIC 5 presents with neonatal onset of normal gammaGT associated cholestasis, elevated serum bilirubin, elevated serum AFP levels, undetectable expression of BSEP in the bile canaliculi, and vitamin K independent coagulopathy. This vitamin K independent coagulopathy is unique to PFIC 5 and has been shown to be a direct result of FXR mutation. Three fibrinogen genes, as well as some coagulation factors have been linked with FXR-dependent induction, which does not occur in PFIC 5 patients. NR1H4/FXR associated PFIC is very rare as only eight cases have been reported in the literature.

### PFIC 6

PFIC 6 (also known as MYO5B deficiency) is caused by a genetic defect in the MYO5B gene located on chromosome 18. The interaction between Myosin 5B (MYO5B) and RAS-related GTP-binding protein 11A (RAB11A) is essential for the polarization of epithelial cells, as well as localizing BSEP to the canalicular membrane. Diminished activity of the MYO5B/RAB11A recycling endosome pathway is related to disrupting the localization of BSEP. Mutations of this gene are associated with microvillus inclusion disease (MVID) which affects the enterocytes and leads to diarrhea, as well as malabsorption. Mislocalized apical brush border proteins, villus atrophy, and the presence of microvillus inclusion bodies are all associated with MVID. Total parenteral nutrition (TPN) is required throughout life, but it has been associated with a high risk of sepsis and small bowel transplant.

MVID has been associated with cholestatic liver disease, which possibly occurs as a result of TPN. In fact, MYO5B gene mutations may account for 20% of the idiopathic low-gammaGT associated cholestasis in pediatric patients. This cholestasis presents with low to normal gammaGT levels, jaundice, pruritus, mildly elevated Alanine transaminase and Aspartate transaminase, elevated serum BS levels, hepatomegaly, portal and lobular fibrosis, and giant cell transformation.

### Other PFIC Subtypes

Phenotypic presentation of PFIC types which have not yet been genetically characterized often present by clinical manifestations similar to other PFIC subtypes described herein. These phenotypes can include jaundice, cholestasis as demonstrated by elevated serum bile acids, elevated liver enzymes, and other symptoms associated with cholestasis, including pruritus, growth deficit, and poor quality of life. New phenotypes associated with mutations in each gene are still emerging.

Intermittent cholestasis or serum bile acid elevations may present in patients with BSEP deficiency or other genetic subtypes. Symptoms associated with intermittent sBa elevations can include pruritus and poot quality of life and can affect both children and adults. Elevations in liver enzymes may be present in patients spontaneously or continuously.

Patients who have undergone a biliary diversion surgery to divert bile acids may still have presentation of symptoms associated with PFIC including pruritus, poor quality of life, as well as poor growth and nutrition. Often surgical diversion which was successful at lowering bile acids and improving symptoms can lose effect in some patients who have recurring serum bile acid elevations and return of pruritus and other associated liver enzyme elevations or symptoms of cholestasis.

In some instances, PFIC mutations may be heterozygous. PFIC 1, PFIC 2, PFIC 3, PFIC 4, PFIC 5, PFIC 6 or any of the other PFIC subtypes may have heterozygosis. By way of a non-limiting example, a subject may have non-truncated PFIC 2 with heterozygosis. In another example, a subject may have PFIC 1 with heterozygosis. In one embodiment, a subject may have heterozygous ABCB11 mutation. In another embodiment, a subject may have heterozygous ATP8B1 mutation.

### ASBT Inhibitors

In various embodiments of methods of the present invention, an ASBT inhibitor is administered to a subject. In some embodiments, the ASBTI inhibitor is maralixibat, or a pharmaceutically acceptable salt thereof. In some embodiments, the ASBTI inhibitor is maralixibat chloride. ASBT inhibitors (ASBTIs) reduce or inhibit bile acid recycling in the distal gastrointestinal (GI) tract, including the distal ileum, the colon and/or the rectum. Inhibition of the apical sodium-dependent bile acid transport interrupts the enterohepatic circulation of bile acids and results in more bile acids being excreted in the feces, see FIG. 1, leading to lower levels of bile acids systemically, thereby reducing bile acid mediated liver damage and related effects and complications. In certain embodiments, the ASBTIs are systemically absorbed. In certain embodiments, the ASBTIs are not systemically absorbed. In one embodiment, maralixibat is a non-systemically absorbed ASBTI. In some embodiments, the ASBTI used in the methods or compositions of the present invention is

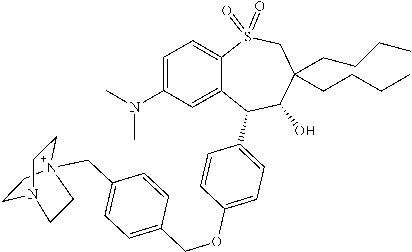 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

maralixibat, or a pharmaceutically acceptable salt thereof.

In one embodiment, the ASBTI used in the methods or compositions of the present invention is

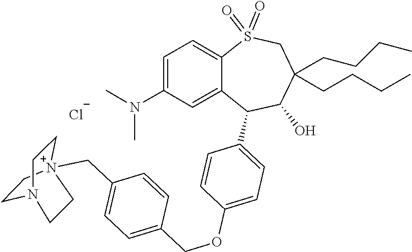 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

(maralixibat chloride, LUM-001, SHP625, lopixibat chloride).

In one embodiment, the ASBTI used in the methods or compositions of the present invention is

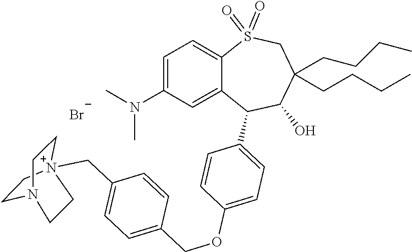 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Br-]]

maralixibat bromide.

In one embodiment, the ASBTI used in the methods or compositions of the present invention is

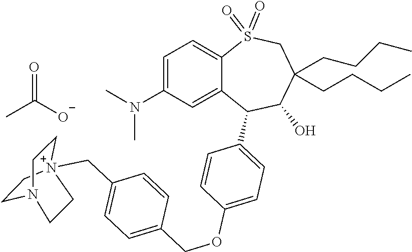

maralixibat acetate.

In one embodiment, the ASBTI used in the methods or compositions of the present invention is

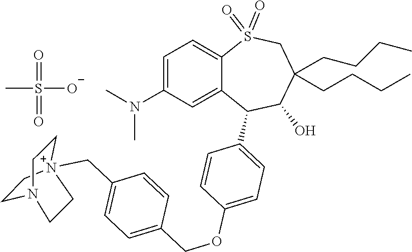

maralixibat mesylate.

### Methods for Treating Cholestasis

Provided herein is a method for treating cholestasis in a subject having a liver disease. The method includes administering to a subject in need of treatment an Apical Sodium-dependent Bile Acid Transporter Inhibitor (ASBTI). The ASBTI is maralixibat, maralixibat chloride, or an alternative pharmaceutically acceptable salt thereof. The ASBTI is administered in an amount of from about 140 μg/kg/day to about 1400 μg/kg/day.

In various embodiments, the liver disease is a cholestatic liver disease. In some embodiments, the liver disease is PFIC, ALGS, PSC, biliary atresia, intrahepatic cholestasis of pregnancy, PBC, any of the cholestatic liver diseases discussed above, or various combinations thereof. In some embodiments, the liver disease is PFIC.

In certain embodiments, the cholestatic liver disease is progressive familial intrahepatic cholestasis (PFIC), PFIC type 1, PFIC type 2, PFIC type 3, PFIC type 4, PFIC type 5, PFIC type 6, Alagille syndrome, Dubin-Johnson Syndrome, biliary atresia, post-Kasai biliary atresia, post-liver transplantation biliary atresia, post-liver transplantation cholestasis, post-liver transplantation associated liver disease, intestinal failure associated liver disease, bile acid mediated liver injury, pediatric primary sclerosing cholangitis, MRP2 deficiency syndrome, neonatal sclerosing cholangitis, a pediatric obstructive cholestasis, a pediatric non-obstructive cholestasis, a pediatric extrahepatic cholestasis, a pediatric intrahepatic cholestasis, a pediatric primary intrahepatic cholestasis, a pediatric secondary intrahepatic cholestasis, BRIC, BRIC type 1, BRIC type 2, BRIC type 3, total parenteral nutrition associated cholestasis, paraneoplastic cholestasis, Stauffer syndrome, drug-associated cholestasis, infection-associated cholestasis, or gallstone disease. In some embodiments, the cholestatic liver disease is a pediatric form of liver disease. In some embodiments, the subject has intrahepatic cholestasis of pregnancy (ICP).

In certain embodiments, the cholestatic liver disease is progressive familial intrahepatic cholestasis (PFIC). In certain embodiments, the cholestatic liver disease is PFIC 1, PFIC 2, PFIC 3, PFIC 4, PFIC 5, or PFIC 6. In one embodiment, the cholestatic liver disease is PFIC 1. In one embodiment, the cholestatic liver disease is PFIC 2. In one embodiment, the cholestatic liver disease is PFIC 3. In one embodiment, the cholestatic liver disease is PFIC 4. In one embodiment, the cholestatic liver disease is PFIC 5. In one embodiment, the cholestatic liver disease is PFIC 6.

In one embodiment, the cholestatic liver disease is non-truncated PFIC 2. In one embodiment, the cholestatic liver disease is truncated PFIC 2.

In one embodiment, the cholestatic liver disease is heterozygous PFIC.

In certain embodiments, a cholestatic liver disease is characterized by one or more symptoms selected from jaundice, pruritus, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and/or renal failure.

In various embodiments the liver disease is PFIC 1 and the subject has a mutation in the ATP8B1 gene. In various embodiments the mutation in the ATP8B1 gene is a missense mutation. In various embodiments the mutation in the ATP8B1 gene is a nonsense mutation. In various embodiments the mutation may be selected from one of the mutations listed in Klomp, et al., “Characterization of mutations in ATP8B1 associated with hereditary cholestasis,” Hepatology, 40:27-38 (2004), which is incorporated herein by reference in its entirety for all purposes.

In various embodiments the liver disease is PFIC 2 and the subject has a non-truncating mutation in the ABCB11 gene. In various embodiments the non-truncating mutation in the ABCB11 gene is a missense mutation. In various embodiments the missense mutation may be selected from one of the mutations listed in Byrne, et al., “Missense Mutations and Single Nucleotide Polymorphisms in ABCB11 Impair Bile Salt Export Pump Processing and Function or Disrupt Pre-Messenger RNA Splicing,” Hepatology, 49:553-567 (2009), which is incorporated herein by reference in its entirety for all purposes.

In various embodiments the liver disease is PFIC 3 and the subject has a mutation in the ABCB4 gene. In various embodiments the mutation in the ABCB4 gene is a missense mutation. In various embodiments the mutation in the ABCB4 gene is a nonsense mutation. In various embodiments the mutation may be selected from one of the mutations listed in Degiorgio, et al., “Molecular characterization and structural implications of 25 new ABCB4 mutations in progressive familial intrahepatic cholestasis type 3 (PFIC 3),” Eur J Hum Genet, 15:1230-1238 (2007), which is incorporated herein by reference in its entirety for all purposes.

In various embodiments the liver disease is PFIC 4 and the subject has a truncating mutation in the TJP2 gene. In various embodiments the truncating mutation in the TJP2 gene may be selected from one of the mutations listed in Sambrotta, et al., “Mutations in TJP2 cause progressive cholestatic liver disease,” Nat Genet, 46:326-328 (2014), which is incorporated herein by reference in its entirety for all purposes.

In various embodiments the liver disease is PFIC 5 and the subject has a mutation in the NR1H4 gene. In various embodiments the mutation in the NR1H4 gene is a nonsense mutation. In various embodiments the mutation may be selected from one of the mutations listed in Gomez-Ospina, et al., “Mutations in the nuclear bile acid receptor FXR cause progressive familial intrahepatic cholestasis,” Nat Commun, 7:1-8 (2016), which is incorporated herein by reference in its entirety for all purposes.

In various embodiments the liver disease is PFIC 6 and the subject has a non-truncating mutation in the MYO5B gene. In various embodiments the non-truncating mutation in the MYO5B gene is a missense mutation. In various embodiments the missense mutation may be selected from one of the mutations listed in Overeem, et al., “A Molecular Mechanism Underlying Genotype-Specific Intrahepatic Cholestasis Resulting From MYO5B Mutations,” Hepatology, 72:213-229 (2020), which is incorporated herein by reference in its entirety for all purposes.

In various embodiments the subject has a condition associated with, caused by or caused in part by a BSEP deficiency. In certain embodiments, the condition associated with, caused by or caused in part by the BSEP deficiency is PFIC 1, PFIC 2, PFIC 3, PFIC 4, PFIC 5, PFIC 6, or a combination thereof. In certain embodiments, the BSEP deficiency is BSEP1, BSEP2, or BSEP3 as defined herein and in van Wessel, et al. “Genotype Correlates with the Natural History of Severe Bile Salt Export Pump Deficiency.” Journal of Hepatology, 73(1):84-93 (2020), which is incorporated herein by reference in its entirety for all purposes. In various embodiments, the subject has heterozygous PFIC. In some embodiments, the subject has PFIC characterized by intermittent cholestasis. In some embodiments, the subject having PFIC has undergone biliary diversion surgery.

In various embodiments, the patient is a pediatric patient under the age of 1 month, 2 months, 3 months, 4 months, 5 months, 6 months, 7 months, 8 months, 9 months, 10 months, 11 months, or 1, 2, 3, 4, 5, 6, 7, 8, 9, 10, 11, 12, 13, 14, 15, 16, 17, or 18 years old. In certain embodiments, the pediatric subject is a newborn, a pre-term newborn, an infant, a toddler, a preschooler, a school-age child, a pre-pubescent child, post-pubescent child, an adolescent, or a teenager under the age of eighteen. In some embodiments, the pediatric subject is a newborn, a pre-term newborn, an infant, a toddler, a preschooler, or a school-age child. In some embodiments, the pediatric subject is a newborn, a pre-term newborn, an infant, a toddler, or a preschooler. In some embodiments, the pediatric subject is a newborn, a pre-term newborn, an infant, or a toddler. In some embodiments, the pediatric subject is a newborn, a pre-term newborn, or an infant. In some embodiments, the pediatric subject is a newborn. In some embodiments, the pediatric subject is an infant. In some embodiments, the pediatric subject is a toddler. In various embodiments, the pediatric patient has PFIC 1, PFIC 2, PFIC 3, PFIC 4, PFIC 5, or PFIC 6. In various embodiments, the pediatric patient has heterozygous PFIC, or PFIC associated with intermittent cholestasis, or the pediatric PFIC subject has undergone biliary diversion surgery. In some embodiments, the patient is an adult over the age of 18, 20, 30, 40, 50, 60, or 70.

In certain embodiments, methods of the present invention comprise non-systemic administration of a therapeutically effective amount of maralixibat or maralixibat chloride. In certain embodiments, the methods comprise contacting the gastrointestinal tract, including the distal ileum and/or the colon and/or the rectum, of an individual in need thereof with maralixibat or maralixibat chloride. In various embodiments, the methods of the present invention cause a reduction in intraenterocyte bile acids, or a reduction in damage to hepatocellular or intestinal architecture caused by cholestasis or a cholestatic liver disease.

In various embodiments, methods of the present invention comprise delivering to ileum or colon of the individual a therapeutically effective amount of maralixibat or maralixibat chloride.

In various embodiments, methods of the present invention comprise reducing damage to hepatocellular or intestinal architecture or cells from cholestasis or a cholestatic liver disease comprising administration of a therapeutically effective amount of maralixibat or maralixibat chloride. In certain embodiments, the methods of the present invention comprise reducing intraenterocyte bile acids/salts through administration of a therapeutically effective amount of maralixibat or maralixibat chloride to an individual in need thereof.

In some embodiments, methods of the present invention provide for inhibition of bile salt recycling upon administration of any of the compounds described herein to an individual. In some embodiments, maralixibat or maralixibat chloride is not absorbed systemically. In some embodiments, maralixibat or maralixibat chloride is administered to the individual orally. In some embodiments, maralixibat or maralixibat chloride is delivered and/or released in the distal ileum of an individual.

In various embodiments, contacting the distal ileum of an individual with an ASBTI (e.g., maralixibat or maralixibat chloride) inhibits bile acid reuptake and increases the concentration of bile acids/salts in the vicinity of L-cells in the distal ileum and/or colon and/or rectum, thereby reducing intraenterocyte bile acids, reducing serum and/or hepatic bile acid levels, reducing overall serum bile acid load, and/or reducing damage to ileal architecture caused by cholestasis or a cholestatic liver disease. Without being limited to any particular theory, reducing serum and/or hepatic bile acid levels ameliorates hypercholemia and/or cholestatic disease.

Administration of a compound described herein may be achieved in any suitable manner including, by way of non-limiting example, by oral, enteric, parenteral (e.g., intravenous, subcutaneous, intramuscular), intranasal, buccal, topical, rectal, or transdermal administration routes. Any compound or composition described herein may be administered in a method or formulation appropriate to treat a newborn or an infant. Any compound or composition described herein may be administered in an oral formulation (e.g., solid or liquid) to treat a newborn or an infant. Any compound or composition described herein may be administered prior to ingestion of food, with food or after ingestion of food.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein may be administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain embodiments of the methods of the present invention, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments of the methods of the present invention, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In various embodiments of the methods of the present invention, a desired dose is conveniently presented in a single dose or in divided doses administered simultaneously (or over a short period of time) or at appropriate intervals, for example as two, three, four or more sub-doses per day. In various embodiments, a single dose of maralixibat or maralixibat chloride is administered every 6 hours, every 12 hours, every 24 hours, every 48 hours, every 72 hours, every 96 hours, every 5 days, every 6 days, or once a week. In some embodiments the total single dose of maralixibat or maralixibat chloride is in a range described below.

In various embodiments of methods of the present invention, in the case wherein the patient's status does improve, upon the doctor's discretion maralixibat or maralixibat chloride is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100% of the original dose, including, by way of example only, 10%, 15%, 20%, 25%, 30%, 35%, 40%, 45%, 50%, 55%, 60%, 65%, 70%, 75%, 80%, 85%, 90%, 95%, or 100% of the original dose. In some embodiments the total single dose of an ASBTI is in a range described below.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

### Dosages

In various embodiments the ASBTI is maralixibat, or a pharmaceutically acceptable salt thereof.

In various embodiments, efficacy and safety of ASBTI administration to the patient is monitored by measuring serum levels of 7α-hydroxy-4-cholesten-3-one (7αC4), sBA concentration, a ratio of 7αC4 to sBA (7αC4:sBA), serum conjugated bilirubin concentration, serum autotaxin concentration, serum fibroblast growth factor (FGF-19) concentration, serum bilirubin concentration, serum total cholesterol concentration, serum LDL-C concentration, serum ALT concentration, serum AST concentration, or a combination thereof. In various embodiments, efficacy of ASBTI administration is measured by monitoring observer-reported itch reported outcome (ITCHRO(OBS)) score, a HRQoL (e.g., PedsQL) score, a CSS score, a xanthoma score, a height Z-score, a weight Z-score, or various combinations thereof. In various embodiments, the method includes monitoring serum levels of 7αC4, sBA concentration, a ratio of 7αC4 to sBA (7αC4:sBA), serum conjugated bilirubin concentration, serum total cholesterol concentration, serum LDL-C concentration, serum autotaxin concentration, serum bilirubin concentration, serum ALT concentration, serum AST concentration, or a combination thereof. In various embodiments, the method includes monitoring observer-reported itch reported outcome (ITCHRO(OBS)) score, a weight Z-score, a HRQoL (e.g., PedsQL) score, a xanthoma score, a CSS score, a height Z-score, or various combinations thereof.

In some embodiments, the ASBTI is administered at a dose of about or at least about 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 280 μg/kg, 300 μg/kg, 250 μg/kg, 280 μg/kg, 300 μg/kg, 400 μg/kg, 500 μg/kg, 560 μg/kg, 600 μg/kg, 700 μg/kg, 800 μg/kg, 900 μg/kg, 1,000 μg/kg, 1,100 μg/kg, 1,200 μg/kg, 1,300 μg/kg, 1,400 μg/kg, 1500 μg/kg, 1,600 μg/kg, 1,700 μg/kg, 1,800 μg/kg, 1,900 μg/kg, or 2,000 μg/kg. In various embodiments, the ASBTI is administered at a dose not exceeding about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 280 μg/kg, 300 μg/kg, 250 μg/kg, 280 μg/kg, 300 μg/kg, 400 μg/kg, 500 μg/kg, 560 μg/kg, 600 μg/kg, 700 μg/kg, 800 μg/kg, 900 μg/kg, 1,000 μg/kg, 1,100 μg/kg, 1,200 μg/kg, 1,300 μg/kg, 1,400 μg/kg, 1,500 μg/kg, 1,600 μg/kg, 1,700 μg/kg, 1,800 μg/kg, 1,900 μg/kg, 2,000, or 2,100 μg/kg. In various embodiments, the ASBTI is administered at a dose of about or of at least about 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1000 mg/day. In various embodiments, the ASBTI is administered at a dose of not more than about 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1,000 mg/day, 1,100 mg/day.

In some embodiments, maralixibat is administered at a dose of from about 140 μg/kg/day to about 1400 μg/kg/day. In various embodiments, maralixibat is administered at a dose of about or at least about 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 250 μg/kg/day, 280 μg/kg/day, 300 μg/kg/day, 400 μg/kg/day, 500 μg/kg/day, 560 μg/kg/day, 600 μg/kg/day, 700 μg/kg/day, 800 μg/kg/day, 900 μg/kg/day, 1,000 μg/kg/day, 1,100 μg/kg/day, 1,200 μg/kg/day, or 1,300 μg/kg/day. In various embodiments, maralixibat is administered at a dose not exceeding about 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 280 μg/kg/day, 300 μg/kg/day, 250 μg/kg/day, 280 μg/kg/day, 300 μg/kg/day, 400 μg/kg/day, 500 μg/kg/day, 560 μg/kg/day, 600 μg/kg/day, 700 μg/kg/day, 800 μg/kg/day, 900 μg/kg/day, 1,000 μg/kg/day, 1,100 μg/kg/day, 1,200 μg/kg/day, 1,300 μg/kg/day, or 1,400 μg/kg/day. In various embodiments, maralixibat is administered at a dose of from about 0.5 μg/kg/day to about 500 μg/kg/day, from about 0.5 μg/kg/day to about 250 μg/kg/day, from about 1 μg/kg/day to about 100 μg/kg/day, from about 10 μg/kg/day to about 50 μg/kg/day, from about 10 μg/kg/day to about 100 μg/kg/day, from about 0.5 μg/kg/day to about 2000 μg/kg/day, from about 280 μg/kg/day to about 1400 μg/kg/day, from about 420 μg/kg/day to about 1400 μg/kg/day, from about 250 to about 550 μg/kg/day, from about 560 μg/kg/day to about 1400 μg/kg/day, from 700 μg/kg/day to about 1400 μg/kg/day, from about 560 μg/kg/day to about 1200 μg/kg/day, from about 700 μg/kg/day to about 1200 μg/kg/day, from about 560 μg/kg/day to about 1000 μg/kg/day, from about 700 μg/kg/day to about 1000 μg/kg/day, from about 800 μg/kg/day to about 1000 μg/kg/day, from about 200 μg/kg/day to about 600 μg/kg/day, from about 300 μg/kg/day to about 600 μg/kg/day, from about 400 μg/kg/day to about 500 μg/kg/day, from about 400 μg/kg/day to about 600 μg/kg/day, from about 400 μg/kg/day to about 700 μg/kg/day, from about 400 μg/kg/day to about 800 μg/kg/day, from about 500 μg/kg/day to about 800 μg/kg/day, from about 500 μg/kg/day to about 900 μg/kg/day, from about 600 μg/kg/day to about 900 μg/kg/day, from about 700 μg/kg/day to about 900 μg/kg/day, from about 200 μg/kg/day to about 600 μg/kg/day, from about 800 μg/kg/day to about 900 μg/kg/day, from about 100 μg/kg/day to about 1500 μg/kg/day, from about 300 μg/kg/day to about 2,000 μg/kg/day, or from about 400 μg/kg/day to about 2000 μg/kg/day.

In some embodiments, maralixibat is administered at a dose of from about 30 μg/kg to about 1400 μg/kg per dose. In some embodiments, maralixibat is administered at a dose of from about 0.5 μg/kg to about 2000 μg/kg per dose, from about 0.5 μg/kg to about 1500 μg/kg per dose, from about 100 μg/kg to about 700 μg/kg per dose, from about 5 μg/kg to about 100 μg/kg per dose, from about 10 μg/kg to about 500 μg/kg per dose, from about 50 μg/kg to about 1400 μg/kg per dose, from about 300 μg/kg to about 2,000 μg/kg per dose, from about 60 μg/kg to about 1200 μg/kg per dose, from about 70 μg/kg to about 1000 μg/kg per dose, from about 70 μg/kg to about 700 μg/kg per dose, from 80 μg/kg to about 1000 μg/kg per dose, from 80 μg/kg to about 800 μg/kg per dose, from 100 μg/kg to about 800 μg/kg per dose, from 100 μg/kg to about 600 μg/kg per dose, from 150 μg/kg to about 700 μg/kg per dose, from 150 μg/kg to about 500 μg/kg per dose, from 200 μg/kg to about 400 μg/kg per dose, from 200 μg/kg to about 300 μg/kg per dose, or from 300 μg/kg to about 400 μg/kg per dose.

In some embodiments, maralixibat is administered at a dose of from about 0.5 mg/day to about 550 mg/day. In various embodiments, maralixibat is administered at a dose of from about 1 mg/day to about 500 mg/day, from about 1 mg/day to about 300 mg/day, from about 1 mg/day to about 200 mg/day, from about 2 mg/day to about 300 mg/day, from about 2 mg/day to about 200 mg/day, from about 4 mg/day to about 300 mg/day, from about 4 mg/day to about 200 mg/day, from about 4 mg/day to about 150 mg/day, from about 5 mg/day to about 150 mg/day, from about 5 mg/day to about 100 mg/day, from about 5 mg/day to about 80 mg/day, from about 5 mg/day to about 50 mg/day, from about 5 mg/day to about 40 mg/day, from about 5 mg/day to about 30 mg/day, from about 5 mg/day to about 20 mg/day, from about 5 mg/day to about 15 mg/day, from about 10 mg/day to about 100 mg/day, from about 10 mg/day to about 80 mg/day, from about 10 mg/day to about 50 mg/day, from about 10 mg/day to about 40 mg/day, from about 10 mg/day to about 20 mg/day, from about 20 mg/day to about 100 mg/day, from about 20 mg/day to about 80 mg/day, from about 20 mg/day to about 50 mg/day, or from about 20 mg/day to about 40 mg/day, or from about 20 mg/day to about 30 mg/day.

In some embodiments, maralixibat is administered twice daily (BID) in an amount of about 150 μg/kg to about 600 μg/kg per dose. In some embodiments, maralixibat is administered in an amount of about 280 μg/kg/day to about 1400 μg/kg/day. In some embodiments, maralixibat is administered in an amount of about 400 μg/kg/day to about 800 μg/kg/day. In some embodiments, maralixibat is administered in an amount of about 20 mg/day to about 50 mg/day. In some embodiments, maralixibat is administered in an amount of from about 5 mg/day to about 15 mg/day. In some embodiments, maralixibat is administered in an amount of from about 560 μg/kg/day to about 1,400 μg/kg/day. In some embodiments, maralixibat is administered in an amount of from about 700 μg/kg/day to about 1,400 μg/kg/day. In some embodiments, maralixibat is administered in an amount of from about 400 μg/kg/day to about 800 μg/kg/day. In some embodiments, maralixibat is administered in an amount of from about 700 μg/kg/day to about 900 μg/kg/day. In some embodiments, maralixibat is administered in an amount of from about 560 μg/kg/day to about 1400 μg/kg/day. In some embodiments, maralixibat is administered in an amount from 700 μg/kg/day to about 1400 μg/kg/day. In some embodiments, maralixibat is administered in an amount of from about 200 μg/kg/day to about 600 μg/kg/day. In some embodiments, maralixibat is administered in an amount of from about 400 μg/kg/day to about 600 μg/kg/day. In some embodiments, maralixibat is administered in an amount of from about 1100 μg/kg/day to about 1200 μg/kg/day. In some embodiments, maralixibat is administered in an amount of about 570 μg/kg/day twice daily (BID) of maralixibat, based on maralixibat free base, which is equivalent to an amount of about 600 μg/kg/day twice daily (BID) of maralixibat chloride.

In various embodiments, the dose of maralixibat is a first dose level. In various embodiments, the dose of maralixibat is a second dose level. In various embodiments, the dose of maralixibat is a third dose level. In various embodiments, the dose of maralixibat is a fourth dose level. In some embodiments, the second dose level is greater than the first dose level. In some embodiments, the second dose level is about or at least about 1.5, 2, 3, 4, 5, 6, 7, 8, 9, 10, 15, 20, 30, 40, 50, 60, 70, 80, 90 or 100 times or fold greater than the first dose level. In some embodiments, the second dose level is not in excess of about 1.5, 2, 3, 4, 5, 6, 7, 8, 9, 10, 15, 20, 30, 40, 50, 60, 70, 80, 90, 100, or 150 times or fold greater than the first dose level. In some embodiments, the third dose level is greater than the second dose level. In some embodiments, the third dose level is about or at least about 1.5, 2, 3, 4, 5, 6, 7, 8, 9, 10, 15, 20, 30, 40, 50, 60, 70, 80, 90 or 100 times or fold greater than the second dose level. In some embodiments, the third dose level is not in excess of about 1.5, 2, 3, 4, 5, 6, 7, 8, 9, 10, 15, 20, 30, 40, 50, 60, 70, 80, 90, 100, or 150 times or fold greater than the second dose level. In some embodiments, the fourth dose level is greater than the third dose level. In some embodiments, the fourth dose level is about or at least about 1.5, 2, 3, 4, 5, 6, 7, 8, 9, 10, 15, 20, 30, 40, 50, 60, 70, 80, 90 or 100 times or fold greater than the third dose level. In some embodiments, the fourth dose level is not in excess of about 1.5, 2, 3, 4, 5, 6, 7, 8, 9, 10, 15, 20, 30, 40, 50, 60, 70, 80, 90, 100, or 150 times or fold greater than the third dose level.

In various embodiments, maralixibat is administered once daily (QD) at one of the above doses or within one of the above dose ranges. In various embodiments, maralixibat is administered twice daily (BID) at one of the above doses or within one of the above dose ranges. In various embodiments, an ASBTI dose is administered daily, every other day, twice a week, or once a week.

In various embodiments, maralixibat is administered regularly for a period of about or of at least about 1, 2, 3, 4, 5, 6, 7, 8, 9, 10, 15, 20, 30, 40, 48, 50, 75, 100, 150, 200, 250, 300, 350, 400, 450, 500, 600, 700, or 800 weeks. In various embodiments, maralixibat is administered for not more than about 1, 2, 3, 4, 5, 6, 7, 8, 9, 10, 15, 20, 30, 40, 48, 50, 75, 100, 150, 200, 250, 300, 350, 400, 450, 500, 600, 700, 800, or 1000 weeks. In various embodiments, maralixibat is administered regularly for a period of about or of at least about 0.5, 1, 1.5, 2, 3, 4, 5, 6, 7, 8, 9, or 10 years. In various embodiments, maralixibat is administered regularly for a period not in excess of about 0.5, 1, 1.5, 2, 3, 4, 5, 6, 7, 8, 9, 10, or 15 years.

Reduction in symptoms or a change in a disease-relevant laboratory measures of cholestatic liver disease

In various embodiments of the above methods of the invention, administration of maralixibat or maralixibat chloride results in a reduction in a symptom or a change in a disease-relevant laboratory measure of the cholestatic liver disease (i.e., improvement in the patient's condition) that is maintained for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years. In various embodiments, the reduction in the symptom or a change in a disease-relevant laboratory measure comprises a reduction in sBA concentration, an increase in serum 7αC4 concentration, an increase in the 7αC4:sBA ratio, an increase in fBA excretion, a reduction in pruritus, a decrease in serum total cholesterol concentration, a decrease in serum LDL-C cholesterol concentration, a reduction in ALT levels, an increase in a quality of life inventory score, an increase in a quality of life inventory score related to fatigue, a reduction in a xanthoma score, a reduction in serum autotaxin concentration, an increase in growth, or a combination thereof. In various embodiments, the reduction in the symptom or a change in a disease-relevant laboratory measure comprises a reduction in sBA concentration, a reduction in pruritus, a decrease in total bilirubin, a decrease in direct bilirubin, an increase in growth, or a combination thereof. In various embodiments, the reduction in the symptom or a change in a disease-relevant laboratory measure is determined relative to a baseline level. That is, the reduction in the symptom or a change in a disease-relevant laboratory measure is determined relative to a measurement of the symptom or a change in a disease-relevant laboratory measure prior to 1) changing a dose level of the ASBTI administered to the patient, 2) changing a dosing regimen followed for the patient, 3) commencing administration of the ASBTI, or 4) any other of various alterations made with the intention of reducing the symptom or a change in a disease-relevant laboratory measure in the patient. In various embodiments, the reduction in symptom or a change in a disease-relevant laboratory measure is a statistically significant reduction.

In various embodiments, the reduction in a symptom or a change in a disease-relevant laboratory measure of the cholestatic liver disease is measured as a progressive decrease in the symptom or a change in a disease-relevant laboratory measure for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years.

In some embodiments, the patient is the pediatric patient and the reduction in symptom or a change in a disease-relevant laboratory measure comprises an increase or improvement in growth. In some embodiments, the increase in growth is measured relative to baseline. In various embodiments, increase in growth is measured as an increase in height Z-score or in weight Z-score. In various embodiments, the increase in height Z-score or in weight Z-score is statistically significant. In various embodiments, the height Z-score, the weight Z-score, or both is increased by at least 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29 0.3, 0.31, 0.32, 0.33, 0.34, 0.35, 0.36, 0.37, 0.38, 0.39 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.7, 0.8, or 0.9 relative to baseline. In some embodiments, the height Z-score, the weight Z-score, or both progressively increases during administration of the ASBTI for a period of about or of at least about 1, 2, 3, 4, 5, 6, 7, 8, 9, 10, 20, 30, 40, 48, 50, 60, 70, or 72 weeks.

In various embodiments, the administration of the ASBTI results in an increase in serum 7αC4 concentration. In various embodiments, the serum 7αC4 concentration is increased by about or at least about 1.5, 2, 3, 4, 5, 6, 7, 8, 9, 10, 15, 20, 30, 40, 50, 60, 70, 80, 90, 100, 200, 300, 400, or 500 times or fold relative to baseline. In various embodiments the serum 7αC4 concentration is increased about or at least about 10%, 20%, 30%, 40%, 50%, 60%, 70%, 80%, 90%, 100%, 150%, 200%, 300%, 400%, 500%, 600%, 700%, 800%, 900%, 1,000%, or 10,000% relative to baseline.

In various embodiments, the administration of the ASBTI results in decrease of total bilirubin. In various embodiments, the total bilirubin is decreased by about or at least about 0.1, 0.2, 0.3, 0.4, 0.5, 0.6, 0.7, 0.8, or 0.9 times or fold relative to baseline. In various embodiments the total bilirubin is decreased by about or at least about 10%, 20%, 30%, 40%, 50%, 60%, 70%, 80%, 90%, or 100% relative to baseline. In various embodiments the total bilirubin is decreased by about or at least about 0.1 mg/dL, 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1.0 mg/dL, 1.1 mg/dL, or 1.2 mg/dL relative to baseline.

In various embodiments, the administration of the ASBTI results in decrease of direct bilirubin. In various embodiments, the direct bilirubin is decreased by about or at least about 0.1, 0.2, 0.3, 0.4, 0.5, 0.6, 0.7, 0.8, or 0.9 times or fold relative to baseline. In various embodiments the direct bilirubin is decreased by about or at least about 10%, 20%, 30%, 40%, 50%, 60%, 70%, 80%, 90%, or 100 relative to baseline. In various embodiments the direct bilirubin is decreased by about or at least about 0.1 mg/dL, 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1.0 mg/dL, 1.1 mg/dL, or 1.2 mg/dL relative to baseline.

In various embodiments, the administration of the ASBTI results in an increase in the 7αC4:sBA ratio to about or by at least about 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold relative to baseline.

In various embodiments, the administration of the ASBTI results in an increase in fBA excretion. In some embodiments, the administration of the ASBTI results in an increase in fBA excretion of about or of at least about 100%, 110%, 115%, 120%, 130%, 150%, 200%, 250%, 275%, 300%, 400%, 500%, 600%, 700%, 800%, 1,000%, 5,000%, 10,000% or 15,000% relative to baseline. In various embodiments, fBA excretion is increased by about or by at least about 1, 1.5, 2, 3, 4, 5, 6, 7, 8, 9, 10, 20, 30, 40, 50, 60, 70, 80, 90, or 100 fold or times relative to baseline. In some embodiments, fBA excretion is increased by about or by at least about 100 μmol, 150 μmol, 200 μmol, 250 μmol, 300 μmol, 400 μmol, 500 μmol, 600 μmol, 700 μmol, 800 μmol, 900 μmol, 1,000 μmol, or 1,500 μmol relative to baseline. In various embodiments, administration of the ASBTI results in a dose-dependent increase in fBA excretion so that administration of a higher dose of the ASBTI results in a corresponding higher level of fBA excretion. In various embodiments, the ASBTI is administered at a dose sufficient to result in an increase in bile acid secretion relative to baseline of at least about or of about 1, 2, 3, 4, 5, 6, 7, 8, 9, 10, 20, 30, 40, 50, 60, 70, 80, 90, or 100 fold or times relative to baseline.

In various embodiments, the administration of the ASBTI results in a decrease in sBA concentration of about or of at least about 5%, 10%, 15%, 20%, 25%, 30%, 31%, 35%, 40%, 45%, 50%, 55%, 57%, 60%, 65%, 70%, 75%, 80%, 85%, 90%, or 95% relative to baseline.

In some embodiments, the administration of the ASBTI results in a reduction in severity of pruritus. In various embodiments, the severity of pruritus is measured using an ITCHRO(OBS) score, an ITCHRO score, a CSS score, or a combination thereof. In various embodiments, the administration of the ASBTI results in a reduction in the ITCHRO(OBS) score on a scale of 1 to 4 of about or of at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 relative to baseline. In various embodiments, the administration of the ASBTI results in a reduction in the ITCHRO score on a scale of 1 to 10 of about or of at least about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or 10. In various embodiments, the administration of the ASBTI results in a reduction of the ITCHRO(OBS) score, the ITCHRO score, or both to zero. In various embodiments, the administration of the ASBTI results in a reduction of the ITCHRO(OBS) score or ITCHRO score to 1.0 or lower. In various embodiments, the administration of the ASBTI results in a reduction of the CSS score by about of at least about 0.1, 0.2, 0.3, 0.4, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 relative to baseline. In various embodiments, the administration of the ASBTI results in a reduction of the CSS score to zero. In various embodiments, the administration of the ASBTI results in a reduction in the CSS score, the ITCHRO(OBS) score, the ITCHRO score, or a combination thereof by about or by at least about 10%, 15%, 20%, 25%, 30%, 35%, 40%, 45%, 50%, 55%, 60%, 65%, 70%, 75%, 80%, 85%, 90%, 95%, or 100% relative to baseline. In various embodiments, a reduced value relative to baseline of the CSS score, the ITCHRO(OBS) score, the ITCHRO score, or a combination thereof is observed on 10%, 20%, 30%, 40%, 50%, 60%, 70%, 75%, 80%, 85%, 90%, 91%, 92%, 93%, 94%, 95%, 96%, 97%, 98%, 99%, or 100% of days.

In some embodiments, patients with a higher baseline ITCHRO(OBS) score demonstrate a greater reduction in the symptom or a change in a disease-relevant laboratory measure than patients having a lower baseline ITCHRO(OBS) score. In some embodiments, patients with a baseline ITCHRO(OBS) score of at least 2, 3, or 4 or an ITCHRO score of at least 4, 5, 6, 7, 8, 9, or 10 have a greater reduction in the symptom or a change in a disease-relevant laboratory measure relative to baseline than a lower reduction in patients having a lower baseline severity of pruritus score. In various embodiments, patients having PSC and baseline ITCHRO scores of at least 4 demonstrate a greater reduction in the symptom or a change in a disease-relevant laboratory measure than patients having a baseline ITCHRO score of less than 4. In various embodiments, the method includes predicting that a patient will have a greater reduction in the symptom or a change in a disease-relevant laboratory measure if a baseline ITCHRO score of the patient is at least 4 as compared to a patient having a baseline ITCHRO score of less than 4. In various embodiments the lower reduction is about or less than about 5%, 10%, 15%, 20%, 25%, 30%, 35%, 40%, 45%, 50%, 55%, or 60% the greater reduction. In various embodiments a difference in the reduction in the symptom or a change in a disease-relevant laboratory measure (i.e., between the greater reduction and the lower reduction) between patients having an ITCHRO score of at least 4 at baseline and patients having an ITCHRO score of less than 4 at baseline is measured at about or at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years following first administration of the ASBTI at the first dose or at the second dose.

In various embodiments, reduction in severity of pruritus caused by administration of the ASBTI to the patient is positively correlated with a reduction in sBA concentration in the patient. In various embodiments, a greater reduction in sBA concentration in the patient correlates with a corresponding greater reduction in severity of pruritus.

In various embodiments, the administration of the ASBTI results in a reduction in serum LDL-C concentration relative to baseline. In some embodiments the serum LDL-C concentration is reduced by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline.

In some embodiments, the administration of the ASBTI results in a reduction in serum total cholesterol concentration relative to baseline. In some embodiments, the administration of the ASBTI results in a reduction in serum LDL-C levels relative to baseline. In some embodiments the serum total cholesterol concentration, the serum LDL-C levels, or both is reduced by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline. In various embodiments, the administration of the ASBTI results in a reduction in serum total cholesterol concentration, of serum LDL-C levels, or both of about or of at least about 1 mg/dL, 2 mg/dL, 3 mg/dL, 4 mg/dL, 5 mg/dL, 10 mg/dL, 12.5 mg/dL, 15 mg/dL, 20 mg/dL, 30 mg/dL, 40 mg/dL or 50 mg/dL relative to baseline.

In various embodiments, the administration of the ASBTI results in a decrease in serum autotaxin concentration. In some embodiments, the administration of the ASBTI results in a reduction in autotaxin concentration of about or of at least about 5%, 10%, 15%, 20%, 25%, 30%, 35%, 40%, 45%, 50%, 55%, 60%, 65%, 70%, 75%, or 80% relative to baseline.

In various embodiments, the administration of the ASBTI results in improvements to sleep. In some embodiments, the sleep is assessed using Exploratory Diary Questionnaire (EDQ(Obs)). In some embodiments the average morning EDQ(Obs) sleep disturbance scores are decreased by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline.

In some embodiments, the administration of the ASBTI results in an improvement in sleep as measured by a reduction of an EDQ(Obs) or EDQ(Pt) score of the subject by at least 1.0 points relative to baseline. In some embodiments, the administration of the ASBTI results in an improvement in sleep as measured by a reduction of an EDQ(Obs) or EDQ(Pt) score of the subject by at least 1.2 points relative to baseline. In some embodiments, the administration of the ASBTI results in an improvement in sleep as measured by a reduction of an EDQ(Obs) or EDQ(Pt) score of the subject by at least 1.4 points relative to baseline. In some embodiments, the administration of the ASBTI results in an improvement in sleep as measured by a reduction of an EDQ(Obs) or EDQ(Pt) score of the subject by at least 1.6 points relative to baseline.

In various embodiments, administration of the ASBTI results in an increase in a quality of life inventory score or in a quality of life inventory score related to fatigue. The quality of life inventory score can be a health-related quality of life (HRQoL) score. In some embodiments, the HRQoL score is a PedsQL score. In various embodiments, the administration of the ASBTI results an increase in the PedsQL score or in a PedsQL score related to fatigue of about or of at least about 5%, 10%, 15%, 20%, 25%, 30%, 45%, or 50% relative to baseline.

In various embodiments, administration of the ASBTI results in a decrease in a xanthoma score relative to baseline. In some embodiments, the xanthoma score is reduced by about or by at least about 2.5%, 5%, 10%, 15%, 20%, 35%, 30%, 35%, 40%, 45%, 50%, 55%, 60%, 65%, 70%, 75%, 80%, 85%, 90%, 95%, or 100% relative to baseline.

In various embodiments, the administration of the ASBTI results in the reduction in the symptom or a change in a disease-relevant laboratory measure by about 1 day, 2 days, 3 days, 4 days, 5 days, 6 days, 7 days, 8 days, 9 days, 10 days, 11 days, 12, days, 13 days, 14 days, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

In various embodiments, serum bilirubin concentration is at pre-administration baseline levels or at normal levels at about or by about 1 day, 2 days, 3 days, 4 days, 5 days, 6 days, 1 week, 2 weeks, 3 weeks, 4 weeks, 1 month, 5 weeks, 6 weeks, 7 weeks, 8 weeks, 2 months, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

In various embodiments, serum ALT concentration is at pre-administration baseline levels or at normal levels at about or by about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year. In some embodiments, the administration of the ASBTI results in a reduction in ALT levels relative to baseline of about or of at least about 1%, 2%, 3%, 4%, 5%, 6%, 7%, 8%, 9%, 10%, 11%, 12%, 13%, 14%, or 15%.

In various embodiments, serum ALT concentration, serum AST concentration, serum bilirubin concentration, serum conjugated bilirubin concentration, or various combinations thereof are within normal range or at pre-administration baseline levels at about or by about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year. In various embodiments, the administration of the ASBTI does not result in a statistically significant change from baseline in serum bilirubin concentration, serum AST concentration, serum ALT concentration, serum alkaline phosphatase concentration, or some combination thereof for a period of at least about or of about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year. In various embodiments, for adult patients with an ITCHRO score of at least 4 at baseline, the administration of the ASBTI does not result in a significant change from baseline in serum conjugated bilirubin concentration for a period of at least about or of about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

### Dose Modulation

In various embodiments, the method includes modulating a dosage of maralixibat administered to the patient. In some embodiments, modulating a dosage of maralixibat may comprise escalating the dosage of maralixibat. In some embodiments, the modulation includes administering maralixibat at a first dose level to the patient for a first week. If the patient tolerates the first dose level, the dose level is increased to a second dose level for a second week. If the patient tolerates the second dose level, the dose level is increased to a third dose level for a third week. If the patient tolerates the third dose level, the dose level is increased to a fourth dose level for the remainder of the treatment study.

In some embodiments, the method comprises a Dose Escalation period. In one non-limiting embodiment, the Dose Escalation period comprises the following weekly steps: a) Dose level 1: 150 μg/kg maralixibat BID for 1 week; b) Dose level 2: 300 μg/kg maralixibat BID for 1 week; c) Dose level 3: 450 μg/kg maralixibat BID for 1 week; d) Dose level 4: 600 μg/kg maralixibat BID for the remaining duration of the administration. In another non-limiting embodiment, dose escalation steps may be delayed or reversed to improve tolerability.

### Pharmaceutical Compositions

In some embodiments, maralixibat is administered as a pharmaceutical composition comprising maralixibat or maralixibat chloride). Any composition described herein can be formulated for ileal, rectal and/or colonic delivery. In more specific embodiments, the composition is formulated for non-systemic or local delivery to the rectum and/or colon. It is to be understood that, as used herein, delivery to the colon includes delivery to sigmoid colon, transverse colon, and/or ascending colon. In still more specific embodiments, the composition is formulated for non-systemic or local delivery to the rectum and/or colon is administered rectally. In other specific embodiments, the composition is formulated for non-systemic or local delivery to the rectum and/or colon is administered orally.

Provided herein, in certain embodiments, is a pharmaceutical composition comprising a therapeutically effective amount of any compound described herein. In certain instances, the pharmaceutical composition comprises an ASBT inhibitor (e.g., maralixibat or maralixibat chloride).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins 1999), all of which references are incorporated herein in their entirety for all purposes.

A pharmaceutical composition, as used herein, refers to a mixture of a compound described herein, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins 1999), all of which references are incorporated herein in their entirety for all purposes.

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Routes of administration, dosage forms, and dosing regimens

In some embodiments, the compositions described herein, and the compositions administered in the methods described herein are formulated to inhibit bile acid reuptake or reduce serum or hepatic bile acid levels. In certain embodiments, the compositions described herein are formulated for rectal or oral administration. In some embodiments, such formulations are administered rectally or orally, respectively. In some embodiments, the compositions described herein are combined with a device for local delivery of the compositions to the rectum and/or colon (sigmoid colon, transverse colon, or ascending colon). In certain embodiments, for rectal administration the composition described herein are formulated as enemas, rectal gels, rectal foams, rectal aerosols, suppositories, jelly suppositories, or retention enemas. In some embodiments, for oral administration the compositions described herein are formulated for oral administration and enteric delivery to the colon.

In certain embodiments, the compositions or methods described herein are non-systemic. In some embodiments, compositions described herein deliver the ASBTI to the distal ileum, colon, and/or rectum and not systemically (e.g., a substantial portion of the enteroendocrine peptide secretion enhancing agent is not systemically absorbed). In some embodiments, oral compositions described herein deliver the ASBTI to the distal ileum, colon, and/or rectum and not systemically (e.g., a substantial portion of the enteroendocrine peptide secretion enhancing agent is not systemically absorbed). In some embodiments, rectal compositions described herein deliver the ASBTI to the distal ileum, colon, and/or rectum and not systemically (e.g., a substantial portion of the enteroendocrine peptide secretion enhancing agent is not systemically absorbed). In certain embodiments, non-systemic compositions described herein deliver less than 90% w/w of the ASBTI systemically. In certain embodiments, non-systemic compositions described herein deliver less than 80% w/w of the ASBTI systemically. In certain embodiments, non-systemic compositions described herein deliver less than 70% w/w of the ASBTI systemically. In certain embodiments, non-systemic compositions described herein deliver less than 60% w/w of the ASBT1 systemically. In certain embodiments, non-systemic compositions described herein deliver less than 50% w/w of the ASBTI systemically. In certain embodiments, non-systemic compositions described herein deliver less than 40% w/w of the ASBTI systemically. In certain embodiments, non-systemic compositions described herein deliver less than 30% w/w of the ASBTI systemically. In certain embodiments, non-systemic compositions described herein deliver less than 25% w/w of the ASBTI systemically. In certain embodiments, non-systemic compositions described herein deliver less than 20% w/w of the ASBTI systemically. In certain embodiments, non-systemic compositions described herein deliver less than 15% w/w of the ASBTI systemically. In certain embodiments, non-systemic compositions described herein deliver less than 10% w/w of the ASBTI systemically. In certain embodiments, non-systemic compositions described herein deliver less than 5% w/w of the ASBTI systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the ASBTI are administered at least twice a day, while in other embodiments the formulations containing the ASBTI are administered at least three times a day. In certain embodiments, the formulations containing the ASBTI are administered up to five times a day. It is to be understood that in certain embodiments, the dosage regimen of composition containing the ASBTI described herein to is determined by considering various factors such as the patient's age, sex, and diet.

The concentration of the ASBTI administered in the formulations described herein ranges from about 1 mM to about 1 M. In certain embodiments the concentration of the ASBTI administered in the formulations described herein ranges from about 1 mM to about 750 mM. In certain embodiments the concentration of the ASBTI administered in the formulations described herein ranges from about 1 mM to about 500 mM. In certain embodiments the concentration of the ASBTI administered in the formulations described herein ranges from about 5 mM to about 500 mM. In certain embodiments the concentration of the ASBTI administered in the formulations described herein ranges from about 10 mM to about 500 mM. In certain embodiments the concentration of the administered in the formulations described herein ranges from about 25 mM to about 500 mM. In certain embodiments the concentration of the ASBTI administered in the formulations described herein ranges from about 50 mM to about 500 mM. In certain embodiments the concentration of the ASBTI administered in the formulations described herein ranges from about 100 mM to about 500 mM. In certain embodiments the concentration of the ASBTI administered in the formulations described herein ranges from about 200 mM to about 500 mM.

In certain embodiments, by targeting the distal gastrointestinal tract (e.g., distal ileum, colon, and/or rectum), compositions and methods described herein provide efficacy (e.g., in reducing microbial growth and/or alleviating symptoms of cholestasis or a cholestatic liver disease) with a reduced dose of enteroendocrine peptide secretion enhancing agent (e.g., as compared to an oral dose that does not target the distal gastrointestinal tract).

### Liquid Dosage Forms

The pharmaceutical liquid dosage forms of the invention may be prepared according to techniques well-known in the art of pharmacy.

A solution refers to a liquid pharmaceutical formulation wherein the active ingredient is dissolved in the liquid. Pharmaceutical solutions of the invention include syrups and elixirs. A suspension refers to a liquid pharmaceutical formulation wherein the active ingredient is in a precipitate in the liquid.

In a liquid dosage form, it is desirable to have a particular pH and/or to be maintained within a specific pH range. In order to control the pH, a suitable buffer system can be used. In addition, the buffer system should have sufficient capacity to maintain the desired pH range. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the liquid dosage form. In one embodiment, the concentration is within the range of 0.005 to 0.5 w/v % in the final liquid dosage form.

The pharmaceutical composition comprising the liquid dosage form of the present invention can also include a suspending/stabilizing agent to prevent settling of the active material. Over time the settling could lead to caking of the active to the inside walls of the product pack, leading to difficulties with redispersion and accurate dispensing. Suitable stabilizing agents include but are not limited to, the polysaccharide stabilizers such as xanthan, guar and tragacanth gums as well as the cellulose derivatives HPMC (hydroxypropyl methylcellulose), methyl cellulose and Avicel RC-591 (microcrystalline cellulose/sodium carboxymethyl cellulose). In another embodiment, polyvinylpyrrolidone (PVP) can also be used as a stabilizing agent.

In addition to the aforementioned components, the ASBTI oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4^{th} Edition, Pharmaceutical Press (2003), which is hereby incorporated by reference in its entirety for all purposes.

Addition of an alternative solvent may help increase solubility of an active ingredient in the liquid dosage form, and consequently the absorption and bioavailability inside the body of a subject. Preferably the alternative solvents include methanol, ethanol or propylene glycol and the like.

In another aspect, the present invention provides a process for preparing the liquid dosage form. The process comprises steps of bringing maralixibat or its pharmaceutically acceptable salts thereof into mixture with the components including glycerol or syrup or the mixture thereof, a preservative, a buffer system and a suspending/stabilizing agent, etc., in a liquid medium. In general, the liquid dosage form is prepared by uniformly and intimately mixing these various components in the liquid medium. For example, the components such as glycerol or syrup or the mixture thereof, a preservative, a buffer system and a suspending/stabilizing agent, etc., can be dissolved in water to form the aqueous solution, then the active ingredient can be then dispersed in the aqueous solution to form a suspension.

In some embodiments, the liquid dosage form provided herein can be in a volume of between about 5 ml to about 50 ml. In some embodiments, the liquid dosage form provided herein can be in a volume of between about 5 ml to about 40 ml. In some embodiments, the liquid dosage form provided herein can be in a volume of between about 5 ml to about 30 ml. In some embodiments, the liquid dosage form provided herein can be in a volume of between about 5 ml to about 20 ml. In some embodiments, the liquid dosage form provided herein can be in a volume of between about 10 ml to about 30 ml. In some embodiments, the liquid dosage form provided herein can be in a volume of about 20 ml. In some embodiments, maralixibat can be in an amount ranging from about 0.001% to about 90% of the total volume. In some embodiments, maralixibat can be in an amount ranging from about 0.01% to about 80% of the total volume. In some embodiments, maralixibat can be in an amount ranging from about 0.1% to about 70% of the total volume. In some embodiments, maralixibat can be in an amount ranging from about 1% to about 60% of the total volume. In some embodiments, maralixibat can be in an amount ranging from about 1% to about 50% of the total volume. In some embodiments, maralixibat can be in an amount ranging from about 1% to about 40% of the total volume. In some embodiments, maralixibat can be in an amount ranging from about 1% to about 30% of the total volume. In some embodiments, maralixibat can be in an amount ranging from about 1% to about 20% of the total volume. In some embodiments, maralixibat can be in an amount ranging from about 1% to about 10% of the total volume. In some embodiments, maralixibat can be in an amount ranging from about 5% to about 70% of the total volume. In some embodiments, maralixibat can be in an amount ranging from about 5% to about 60% of the total volume. In some embodiments, maralixibat can be in an amount ranging from about 5% to about 50% of the total volume. In some embodiments, maralixibat can be in an amount ranging from about 5% to about 40% of the total volume. In some embodiments, maralixibat can be in an amount ranging from about 5% to about 30% of the total volume. In some embodiments, maralixibat can be in an amount ranging from about 5% to about 20% of the total volume. In some embodiments, maralixibat can be in an amount ranging from about 5% to about 10% of the total volume. In some embodiments, maralixibat can be in an amount ranging from about 10% to about 50% of the total volume. In some embodiments, maralixibat can be in an amount ranging from about 10% to about 40% of the total volume. In some embodiments, maralixibat can be in an amount ranging from about 10% to about 30% of the total volume. In some embodiments, maralixibat can be in an amount ranging from about 10% to about 20% of the total volume. In one embodiment, the resulted liquid dosage form can be in a liquid volume of 10 ml to 30 ml, preferably 20 ml, and the active ingredient can be in an amount ranging from about 0.001 mg/ml to about 25 mg/ml, or from about 0.025 mg/ml to about 8 mg/ml, or from about 0.1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 10 mg/ml, or about 12 mg/ml, or about 14 mg/ml or about 16 mg/ml, or about 18 mg/ml, or about 20 mg/ml, or about 25 mg/ml. In one embodiment, the active ingredient is maralixibat present in an amount of 9.5 mg/ml. In one embodiment, the active ingredient is maralixibat chloride present in an amount of 10 mg/ml.

### Oral Solution

In some embodiments, the pharmaceutical composition is formulated as an oral solution comprising maralixibat chloride, a preservative, an antioxidant, a flavoring agent, a sweetener, and water.

### Preservative

In certain embodiments, the compositions of the present invention comprise a preservative. In certain embodiments, the preservative is an antimicrobial preservative.

In certain embodiments, the antimicrobial preservative is selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

In certain embodiments, the preservative is propylene glycol.

In certain embodiments, the preservative is present in an amount of at least about 10% w/w of the composition. In certain embodiments, the preservative is present in an amount of at least about 20% w/w of the composition. In certain embodiments, the preservative is present in an amount of at least about 25% w/w of the composition. In certain embodiments, the preservative is present in an amount of at least about 30% w/w of the composition.

In certain embodiments, the preservative is present in an amount of from about 30% to about 40% of the composition.

In certain embodiments, the preservative is present in an amount of from about 32% to about 37% of the composition. In certain embodiments, the preservative is present in an amount of from about 33% to about 36% of the composition.

In certain embodiments, the preservative is present in an amount of about 33% of the composition. In certain embodiments, the preservative is present in an amount of about 34% of the composition. In certain embodiments, the preservative is present in an amount of about 35% of the composition.

### Antioxidant

In certain embodiments, the compositions of the present invention comprise an antioxidant. In certain embodiments, the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

In certain embodiments, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid) In certain embodiments, the antioxidant is EDTA.

In certain embodiments, the antioxidant is present in an amount of about 0.001% to about 1% w/w of the composition. In certain embodiments, the antioxidant is present in an amount of about 0.005% to about 0.75% w/w of the composition. In certain embodiments, the antioxidant is present in an amount of about 0.01% to about 0.5% w/w of the composition. In certain embodiments, the antioxidant is present in an amount of about 0.05% to about 0.25% w/w of the composition. In certain embodiments, the antioxidant is present in an amount of about 0.075% to about 0.2% w/w of the composition. In certain embodiments, the antioxidant is present in an amount of about 0.1% w/w of the composition.

In some embodiments, the pharmaceutical composition comprises from about 5 mg/mL to about 50 mg/mL of maralixibat chloride; from about 300 mg/mL to about 400 mg/mL of propylene glycol; about 1 mg/mL of disodium EDTA; a sweetener, a flavoring agent, or a combination thereof, and water.

In some embodiments, the pharmaceutical composition comprises from about 5 mg/mL to about 50 mg/mL of maralixibat chloride; from about 300 mg/mL to about 400 mg/mL of propylene glycol; about 1 mg/mL of disodium EDTA; about 10 mg/mL sucralose, about 5 mg/mL grape flavor, and water.

In some embodiments, the pharmaceutical composition comprises about 10 mg/mL of maralixibat chloride; about 360 mg/mL of propylene glycol; about 1 mg/mL of disodium EDTA; about 10 mg/mL sucralose, about 5 mg/mL grape flavor, and water.

### Pediatric Dosage Formulations and Compositions

Provided herein, in certain embodiments, is a pediatric dosage formulation or composition comprising a therapeutically effective amount of any compound described herein. In certain instances, the pharmaceutical composition comprises an ASBT inhibitor (e.g., maralixibat or maralixibat chloride).

In certain embodiments, suitable dosage forms for the pediatric dosage formulation or composition include, by way of non-limiting example, aqueous or non-aqueous oral dispersions, liquids, gels, syrups, elixirs, slurries, suspensions, solutions, controlled release formulations, fast melt formulations, effervescent formulations, lyophilized formulations, chewable tablets, gummy candy, orally disintegrating tablets, powders for reconstitution as suspension or solution, sprinkle oral powder or granules, dragees, delayed release formulations, extended release formulations, pulsatile release formulations, multiparticulate formulations, and mixed immediate release and controlled release formulations. In some embodiments, provided herein is a pharmaceutical composition wherein the pediatric dosage form is selected from a solution, syrup, suspension, elixir, powder for reconstitution as suspension or solution, dispersible/effervescent tablet, chewable tablet, gummy candy, lollipop, freezer pops, troches, oral thin strips, orally disintegrating tablet, orally disintegrating strip, sachet, and sprinkle oral powder or granules.

In another aspect, provide herein is a pharmaceutical composition wherein at least one excipient is a flavoring agent or a sweetener. In some embodiments, provided herein is a coating. In some embodiments, provided herein is a taste-masking technology selected from coating of drug particles with a taste-neutral polymer by spray-drying, wet granulation, fluidized bed, and microencapsulation; coating with molten waxes of a mixture of molten waxes and other pharmaceutical adjuvants; entrapment of drug particles by complexation, flocculation or coagulation of an aqueous polymeric dispersion; adsorption of drug particles on resin and inorganic supports; and solid dispersion wherein a drug and one or more taste neutral compounds are melted and cooled, or co-precipitated by a solvent evaporation. In some embodiments, provided herein is a delayed or sustained release formulation comprising drug particles or granules in a rate controlling polymer or matrix.

Suitable sweeteners include sucrose, glucose, fructose or intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, xylitol, cyclamate, neomate, neohesperidine dihydrochalcone or mixtures thereof, thaumatin, palatinit, stevioside, rebaudioside, Magnasweet®. The total concentration of the sweeteners may range from effectively zero to about 300 mg/ml based on the liquid composition upon reconstitution.

In order to increase the palatability of the liquid composition upon reconstitution with an aqueous medium, one or more taste-making agents may be added to the composition in order to mask the taste of the ASBT inhibitor. A taste-masking agent can be a sweetener, a flavoring agent or a combination thereof. The taste-masking agents typically provide up to about 0.1% or 5% by weight of the total pharmaceutical composition. In a preferred embodiment of the present invention, the composition contains both sweetener(s) and flavor(s).

A flavoring agent herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of flavoring agents and/or sweeteners useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. Flavoring agents can be used singly or in combinations of two or more. In some embodiments, the aqueous liquid dispersion comprises a sweetening agent or flavoring agent in a concentration ranging from about 0.001% to about 5.0% the volume of the aqueous dispersion. In one embodiment, the aqueous liquid dispersion comprises a sweetening agent or flavoring agent in a concentration ranging from about 0.001% to about 1.0% the volume of the aqueous dispersion. In another embodiment, the aqueous liquid dispersion comprises a sweetening agent or flavoring agent in a concentration ranging from about 0.005% to about 0.5% the volume of the aqueous dispersion. In yet another embodiment, the aqueous liquid dispersion comprises a sweetening agent or flavoring agent in a concentration ranging from about 0.01% to about 1.0% the volume of the aqueous dispersion. In yet another embodiment, the aqueous liquid dispersion comprises a sweetening agent or flavoring agent in a concentration ranging from about 0.01% to about 0.5% the volume of the aqueous dispersion.

In certain embodiments, a pediatric pharmaceutical composition described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” for pediatric pharmaceutical compositions includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), all of which references are incorporated herein by reference in their entirety for all purposes.

Moreover, in certain embodiments, the pediatric pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In certain aspects, the pediatric composition or formulation containing one or more compounds described herein is orally administered for local delivery of maralixibat, maralixibat chloride, or other compounds described herein to the colon and/or rectum. Unit dosage forms of such compositions include a pill, tablet or capsules formulated for enteric delivery to colon. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry starch, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starches, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

In some embodiments, maralixibat, maralixibat chloride, or other compounds described herein are orally administered in association with a carrier suitable for delivery to the distal gastrointestinal tract (e.g., distal ileum, colon, and/or rectum).

In certain embodiments, a pediatric composition described herein comprises maralixibat, maralixibat chloride, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pediatric pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In further embodiments, a tablet or capsule comprising an ASBTI or other compounds described herein is film-coated for delivery to targeted sites within the gastrointestinal tract. Examples of enteric film coats include and are not limited to hydroxypropylmethylcellulose, polyvinyl pyrrolidone, hydroxypropyl cellulose, polyethylene glycol 3350, 4500, 8000, methyl cellulose, pseudo ethylcellulose, amylopectin and the like.

### Solid Dosage Forms for Pediatric Administration

Solid dosage forms for pediatric administration of the present invention can be manufactured by standard manufacturing techniques. Non-limiting examples of oral solid dosage forms for pediatric administration are described below.

### Effervescent Compositions

The effervescent compositions of the invention may be prepared according to techniques well-known in the art of pharmacy.

Effervescent formulations contain and effervescent couple of a base component and an acid component, which components reach in the presence of water to generate a gas. In some embodiments, the base component may comprise, for example, an alkali metal or alkaline earth metal carbonate, or bicarbonate. The acid component may comprise, for example, an aliphatic carboxylic acid or a salt thereof, such as citric acid. The base and acid components may each independently constitute, for example, 25% to 55% (w/w) of the effervescent composition. The ratio of acid component to base component may be within the range of 1:2 to 2:1.

The effervescent compositions of the invention may be formulated using additional pharmaceutically acceptable carriers or excipients as appropriate. For example, one or more taste masking agents may be used. Dyes may also be used, as pediatric patients often prefer colorful pharmaceutical combinations. The compositions may take the form of, for example, tablets, granules or powders, granules or powders presented in a sachet.

### Chewable Tablets

The chewable tablets of the invention may be prepared according to techniques well-known in the art of pharmacy.

Chewable tablets are tablets that are intended to disintegrate in the mouth under the action of chewing or sucking and where, in consequence, the active ingredient has greater opportunity to come into contact with the bitter-taste receptors on the tongue.

One method of overcoming this issue is to absorb the active ingredient onto a suitable substrate. This approach is known in the art and described for example in U.S. Pat. No. 4,647,459, which is incorporated herein by reference in its entirety for all purposes.

Another approach involves forming the active ingredient into an aggregate along with a pre-swelled substantially anhydrous hydrocolloid. The hydrocolloid absorbs saliva and acquires a slippery texture which enables it to lubricate the particles of aggregate and mask the taste of the active ingredient. This approach is known in the art and described for example in European patent application 0190826, which is incorporated herein by reference in its entirety for all purposes.

Another approach involves employing a water-insoluble hygroscopic excipient such as microcrystalline cellulose. This approach is known in the art and described for example in U.S. Pat. No. 5,275,823, which is incorporated herein by reference in its entirety for all purposes.

In addition to the above approaches, the chewable tablets of the present invention can also contain other standard tableting excipients such as a disintegrant and a taste-masking agent.

### Orodispersible Tablets

The orodispersible tablets of the invention may be prepared according to techniques well-known in the art of pharmacy.

In orodispersible tablets of the invention, the excipient mixtures are such as to provide it with a disintegration rate so that its disintegration in the buccal cavity occurs in an extremely short time and especially shorter than sixty seconds. In some embodiments, the excipient mixture is characterized by the fact that the active substance is in the form of coated or non-coated microcrystals of microgranules. In some embodiments, the orodispersible tablet comprises one or several disintegrating agents of the carboxymethylcellulose type or insoluble reticulated PVP type, one or several swelling agents which may comprise a carboxymethylcellulose, a starch, a modified starch, or a microcrystalline cellulose or optionally a direct compression sugar.

### Powders for Reconstitution

The powder for reconstitution pharmaceutical compositions of the invention may be prepared according to techniques well-known in the art of pharmacy.

In some embodiments, the powder for reconstitution compositions of the invention comprise an effective amount of at least one internal dehydrating agent. The internal dehydrating agent can enhance the stability of the powder. In some embodiments, the internal dehydrating agent is magnesium citrate or disodium carbonate. In some embodiments, the powder composition comprises a pharmaceutically acceptable diluents, such as sucrose, dextrose, mannitol, xylitol, or lactose.

Powder compositions of the inventions may be placed in sachets or bottles for contemporaneous dissolution or for short term storage in liquid form (e.g. 7 days).

### Gummy Candies

The gummy candies of the invention may be prepared according to techniques well-known in the art of pharmacy.

Traditional gummy candy is made from a gelatin base. Gelatin gives the candy its elasticity, the desired chewy consistency, and a longer shelf life. In some embodiments, the gummy candy pharmaceutical composition of the invention includes a binding agent, a sweetener, and an active ingredient.

In some embodiments, the binding agent is a pectin gel, gelatin, food starch, or any combination thereof.

In some embodiments, the gummy candy comprises sweeteners, a binding agent, natural and/or artificial flavors and colors and preservatives. In some embodiments, the gummy candy comprises glucose syrup, natural cane juice, gelatin, citric acid, lactic acid, natural colors, natural flavors, fractionated coconut oil, and carnauba wax.

An ASBT inhibitor (e.g., maralixibat) may be used in the preparation of medicaments for the prophylactic and/or therapeutic treatment of cholestasis or a cholestatic liver disease (e.g., PFIC). A method for treating any of the diseases or conditions described herein in an individual in need of such treatment, may involve administration of pharmaceutical compositions containing at least one ASBT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

### EXAMPLES

The following examples are provided to further describe some of the embodiments disclosed herein. The examples are intended to illustrate, not to limit, the disclosed embodiments.

### Example 1. Randomized Double-Blind Placebo-Controlled Phase 3 Clinical Study to Evaluate the Efficacy and Safety of Maralixibat in the Treatment of Subjects with PFIC

### Study Overview

The primary analysis was conducted in the intent-to-treat (ITT) population of subjects with documented biallelic mutations in ABCB11 (PFIC 2), based on standard-of-care genotyping (primary cohort criteria). Subjects with a predicted complete absence of bile salt excretion pump (BSEP) function based on the type of ABCB11 mutation (PFIC 2), as determined by a standard-of-care genotype, were excluded from the primary cohort and could enroll only in the supplemental cohort. Subjects with other PFIC subtypes (e.g., PFIC 1/3/4/5/6, or new PFIC mutation variants) or postsurgical subjects (e.g., after internal or external biliary diversion surgery or subjects with reversal of biliary diversion surgery) were enrolled in a separate supplemental cohort and evaluated as part of secondary and exploratory analyses.

Subjects in the primary cohort were randomized in a 1:1 ratio to receive maralixibat or placebo. Subjects in the supplemental cohort were randomized in a 1:1 ratio to receive maralixibat or placebo within the following subcohorts: a) PFIC 1; b) PFIC 3; c) all other supplemental cohort subjects. All subjects received study treatment in addition to standard-of-care therapy, including antipruritic concomitant treatment.

In the primary cohort, 31 subjects were enrolled in order to achieve at least 26 subjects completing the study. In the supplemental cohort, overall enrollment was 62 subjects.

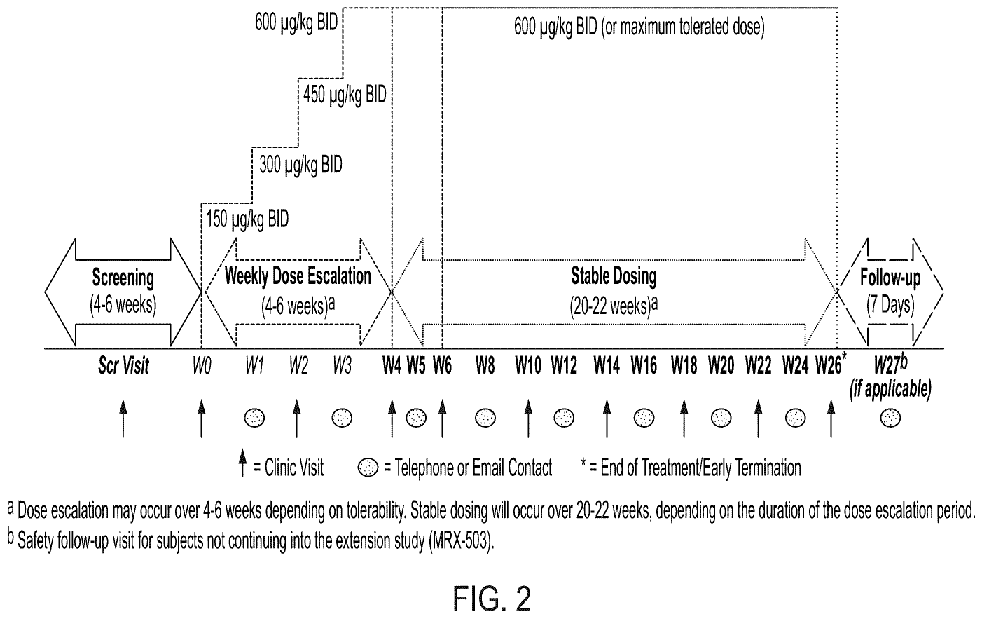

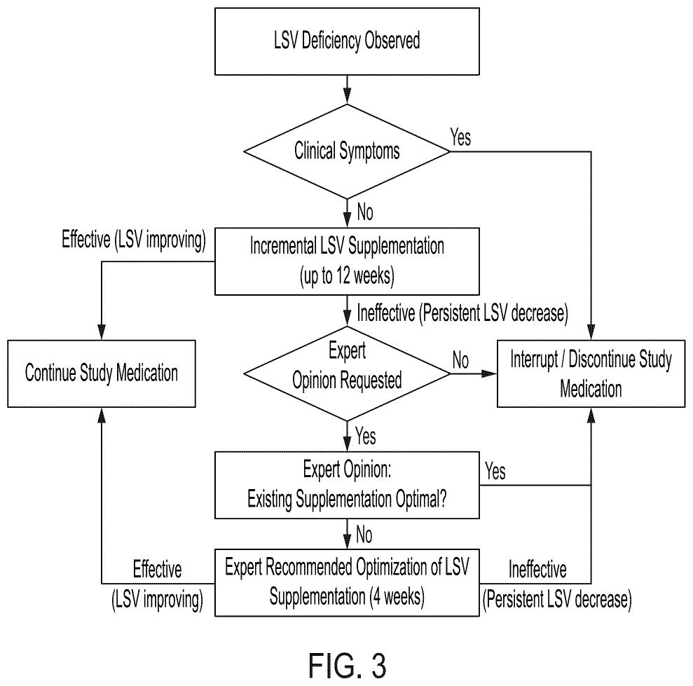

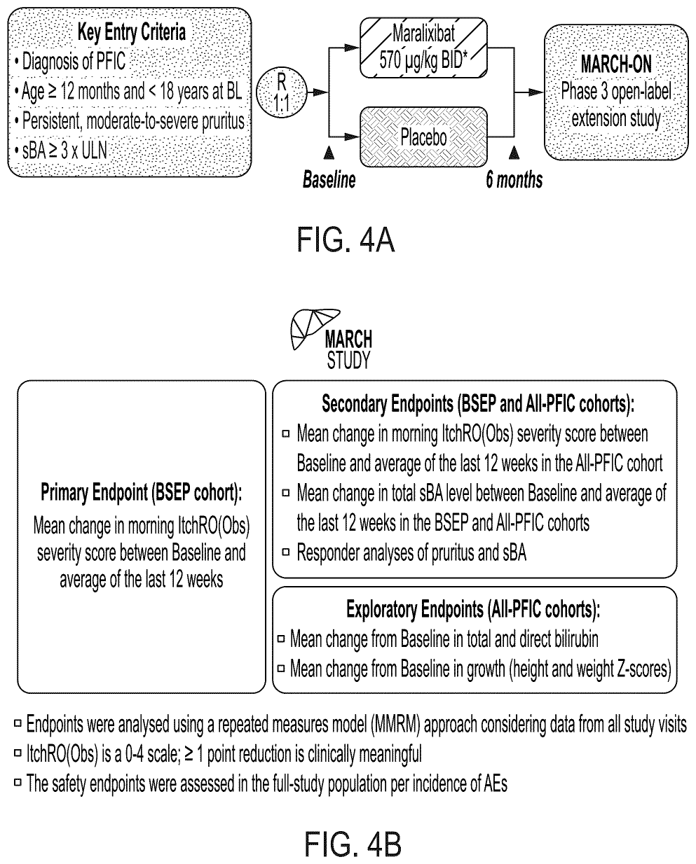

An overview of the Phase 3 clinical study is summarized in FIG. 2. The study was divided into 4 parts: 1) Screening period (up to 6 weeks); 2) Dose Escalation period (4-6 weeks); 3) Stable Dosing period (20-22 weeks); 4) Safety Follow-up period (7 days).

### Study Population

Key inclusion criteria for the Phase 3 clinical study were the following: 1) Subjects aged 1-18 years with a body weight ≥5.0 kg at time of baseline; 2) Cholestasis as manifested by total sBA ≥3×ULN; 3) An average AM ItchRO(Obs) score ≥1.5 during 4 consecutive weeks of the screening period, leading to the baseline visit; 4) Completion of at least 21 valid morning ItchRO(Obs) entries during 4 consecutive weeks of the screening period, leading to the baseline visit; 5) Diagnosis of PFIC based on: Chronic cholestasis as manifested by persistent (>6 months) pruritus in addition to biochemical abnormalities and/or pathological evidence of progressive liver disease and: a) Primary Cohort: Subjects with genetic testing results consistent with biallelic disease-causing variation in ABCB11 (PFIC 2), based on standard-of-care genotyping; b) Supplemental Cohort: i) Subjects with genetic testing results consistent with biallelic disease-causing variation in ATP8B1 (PFIC 1), ABCB4 (PFIC 3), or TJP2 (PFIC 4), based on standard-of-care genotyping; ii) Subjects with PFIC phenotype without a known mutation or with another known mutation not described above or with intermittent cholestasis as manifested by fluctuating sBA levels; iii) Subjects with PFIC after internal or external biliary diversion surgery or for whom internal or external biliary diversion surgery was reversed.

A valid entry is an entry completed and not answered as “I don't know”; maximum allowed invalid reports are 7, with no more than 2 invalid reports during the last 7 days before randomization.

Key exclusion criteria for the Phase 3 study were the following: 1) Predicted complete absence of bile salt excretion pump (B SEP) function based on the type of ABCB11 mutation (PFIC 2), as determined by a standard-of-care genotyping (applies to primary cohort only); 2) Recurrent intrahepatic cholestasis, indicated by a history of sBA levels <3× ULN or intermittent pruritus (applies to primary cohort only); 3) Current or recent history (<1 year) of atopic dermatitis or other non-cholestatic diseases associated with pruritus; 4) History of surgical disruption of the enterohepatic circulation (applies to primary cohort only); 5) Chronic diarrhea requiring intravenous fluid or nutritional intervention for the diarrhea and/or its sequelae at screening or during the 6 months prior to screening; 6) Previous or need for imminent liver transplant; 7) Decompensated cirrhosis (international normalized ratio [INR]>1.5, and/or albumin <30 g/L, history or presence of clinically significant ascites, and/or variceal hemorrhage, and/or encephalopathy); 8) ALT or TSB>15× ULN at screening; 9) Presence of other liver disease; 10) Presence of any other disease or condition known to interfere with the absorption, distribution, metabolism, or excretion of drugs, including bile salt metabolism in the intestine (e.g., inflammatory bowel disease), per investigator discretion; 11) Possible malignant liver mass on imaging, including screening ultrasound; 12) Known diagnosis of human immunodeficiency virus (HIV) infection; 13) Any prior cancer diagnosis (except for in situ carcinoma) within 5 years before the screening visit; 14) Any known history of alcohol or substance abuse; 15) Administration of bile acids or lipid binding resins or phenylbutyrates during the screening period; 16) Administration of any investigational drug, biologic, or medical device during the screening period; 17) Previous use of an ileal bile acid transporter inhibitor (IBATi); 18) History of nonadherence to medical regimens, unreliability, medical condition, mental instability or cognitive impairment that, in the opinion of the investigator or sponsor medical monitor, could compromise the validity of informed consent, compromise the safety of the subject, or lead to nonadherence with the study protocol or inability to conduct the study procedures; 19) Known hypersensitivity to maralixibat or any of its excipients.

### Study Medication

The study medication administered was maralixibat chloride, provided as an oral solution form (e.g., 5, 10, 15, and 20 mg/mL) along with either 0.5, 1.0, or 3.0-mL sized dosing dispensers. The reference/comparator product was a placebo which was also provided as an oral solution along with dosing dispensers of the same size (either 0.5 mL, 1.0 mL, or 3.0 mL). Maralixibat and placebo (the study medication) were presented in 30-mL volumes packaged in 30-mL size amber colored PET bottles and require refrigerated storage conditions (2° C.-8° C.).

One of the excipients in the maralixibat oral solution is propylene glycol (PG); in order to limit subject exposure to PG, a specific strength of oral solution was prescribed to a given subject based on body weight and target dose. The dosing plan limited PG exposure to ≤26 mg/kg/day and, at the same time, provided reasonable (not too high or too low) dosing volumes to ensure accurate dosing.

The placebo solution contained all components of the study medication except the active drug substance. All packaged study medication components, including the dosing dispensers, were identical in order to maintain the blind.

### Administration of Study Medication

An interactive response technology (IRT) was used for screening and enrolling subjects, randomization, study medication supply dispensation and management, inventory management and supply ordering, study medication expiration tracking and management, and emergency unblinding. Individual subject treatment was automatically assigned by the IRT. Subjects were randomized after confirmation of study eligibility in a 1:1 ratio via a computer-generated randomization schedule to receive maralixibat or placebo, stratified by cohort.

Subjects were administered (or self-administered) varying volumes of ready-to-use oral solution study medication at each dosing visit, starting with the baseline visit (Visit 1). The dosing volume is determined based on the individual body weight, the dose level according to the dose escalation plan (150, 300, 450, or 600 μg/kg), and the strength of solution being administered (5, 10, 15, or 20 mg/mL).

Study medication administration took place during the Dose Escalation and Stable Dosing periods of the study based on a BID regimen. The morning dose was administered approximately 30 minutes before breakfast and the evening dose approximately 30 minutes before the main evening meal. Study medication was administered approximately at the same time each day throughout the study.

For subjects assigned to maralixibat, the Dose Escalation period consisted of the following weekly steps: Dose level 1, 150 μg/kg maralixibat BID for 1 week; Dose level 2, 300 μg/kg maralixibat BID for 1 week; Dose level 3, 450 μg/kg maralixibat BID for 1 week; Dose level 4, 600 μg/kg maralixibat BID for the remaining duration of the study.

### Study Schedule

The study procedures and assessments performed throughout the study can be found in the schedule of assessments in Table 1.

Genetic Testing Results. ATP8B1 (PFIC 1), ABCB11 (PFIC 2), ABCB4 (PFIC 3), TJP2 (PFIC 4), NR1H4 (PFIC 5), and MYO5B (PFIC 6) mutations are predictive of PFIC. Standard of care genotyping results were reviewed and documented by the sponsor or designee for confirmation of PFIC subtype and for determination of cohort assignment.

Efficacy. Severity of pruritus was assessed using the Itch caregiver/patient reported outcome measure (ItchRO™) administered as a twice daily electronic diary. Caregivers for all subjects completed the Observer instrument: ItchRO(Obs). The ItchRO(Obs) was completed by the same caregiver for consistency, whenever possible. Only subjects ≥9 years of age at screening completed the patient instrument: ItchRO(Pt). If a subject turned 9 years of age at any point after screening, they did not complete the ItchRO(Pt). Pruritus was assessed and recorded twice daily, via ItchRO, beginning with the day after the screening visit and every day throughout the duration of the study. The severity of pruritus was measured by the completion of the first question in the ItchRO(Obs) (how severe were your child's itch-related symptoms) or ItchRO(Pt) (how itchy did you feel). The frequency of pruritus was measured by completion of the third question in the ItchRO(Obs) (how much of the time was your child rubbing or scratching) or ItchRO(Pt) (how much of last night/today did feeling itchy make you rub or scratch). Caregivers and subjects rated the severity and frequency of pruritus using 5 choices to describe their itching condition. ItchRO(Pt) and ItchRO(Obs) have been previously described in Kamath, et al., “Development of a Novel Tool to Assess the Impact of Itching in Pediatric Cholestasis,” Patient, 11:69-82 (2018), which is hereby incorporated by reference in its entirety for all purposes.

TABLE 1
Schedule of Assessments

| | Procedures | | | | | | | | |
|---|---|---|---|---|---|---|---|---|---|
| | Screening^{a} | Dose Escalation (4-6 weeks) | | | | | | Stable Dosing (20-22 weeks) | |
| | Visit/Subject Contact # | | | | | | | | |
| | Screening/ | Baseline/ | Subject | | Subject | | Subject | | Subject |
| | V0 | V1 | contact | V2 | contact | V3 | contact | V4 | contact |
| | Study Week | | | | | | | | |
| | −6 | 0 | 1 | 2 | 3 | 4 | 5 | 6 | 8 |
| | Study Day | | | | | | | | |
| | Day −42 | 0 | 7 | 14 | 21 | 28 | 35 | 42 | 56 |
| | Window (in days) | | | | | | | | |
| | (−5) | | (±2) | (±2) | (±2) | (±2) | (±2) | (±2) | (±2) |
| Informed Consent/Assent | X | | | | | | | | |
| Eligibility Assessment | X | X | | | | | | | |
| Demographics | X | | | | | | | | |
| Medical History | X | | | | | | | | |
| Physical & Vital signs^{c} | X | X | | X | | X | | X | |
| ECG | X | X | | | | | | | |
| Liver Ultrasound^{d} | X | | | | | | | | |
| Pregnancy Test^{e} | S | U | | | | U | | U | |
| Confirm PFIC Genotype | X | | | | | | | | |
| Results^{f} | | | | | | | | | |
| eDiary Device Provided/ | P | | | | | | | | |
| Returned^{g} | | | | | | | | | |
| ItchRO(Obs), ItchRO(Pt) | Twice-daily completion of ItchRO & EDQ | | | | | | | | |
| & EDQ | | | | | | | | | |
| Review eDiary & Assess | | X | | X | | X | | X | |
| Compliance | | | | | | | | | |
| Randomized | | X | | | | | | | |
| CIS^{h} | | X | | | | X | X | X | |
| PISⁱ | | X | | | | X | X | X | |
| PIC | | | | | | | | X | |
| CIC | | | | | | | | X | |
| PedsQL^{j} | | X | | | | X | | X | |
| Clinician Scratch Scale | X | X | | X | | X | | X | |
| CBC with Differential | X | X | | X | | | | X | |
| Coagulation | X | X | | X | | | | X | |
| Chemistry Panel | X | X | | X | | | | X | |
| Lipid Panel^{k} | X | X | | X | | | | X | |
| Urinalysis | | X | | | | | | X | |
| FGF-19, Autotaxin, C4^{k} | | X | | | | | | X | |
| sBA collection^{k} | X | X | | X | | | | X | |
| Lipid Soluble Vitamins^{k,l} | | X | | | | | | X | |
| AFP Sample | | X | | | | | | | |
| PK Sample^{m} | | | | | | | | | |
| Serum storage sample | | X | | X | | | | X | |
| Healthcare Utilization | | | | X | | | | X | |
| Study Medication | | X | | X | | X | | X | |
| Suppliedⁿ | | | | | | | | | |
| Study Medication | | Twice-daily | | | | | | | |
| Administration^{o} | | administration | | | | | | | |
| Assess AEs | | X | X | X | X | X | X | X | X |
| Prior and Concomitant | X | X | X | X | X | X | X | X | X |
| Treatments | | | | | | | | | |

| | Procedures | | | | | | | | | |
|---|---|---|---|---|---|---|---|---|---|---|
| | Stable Dosing (20-22 weeks) | | | | | | | | | |
| | Visit/Subject Contact # | | | | | | | | | |
| | | Subject | | Subject | | Subject | | Subject | EOT/ET | Follow- |
| | V5 | contact | V6 | contact | V7 | contact | V8 | contact | V9^{b} | up |
| | Study Week | | | | | | | | | |
| | 10 | 12 | 14 | 16 | 18 | 20 | 22 | 24 | 26 | 27 |
| | Study Day | | | | | | | | | |
| | 70 | 84 | 98 | 112 | 126 | 140 | 154 | 168 | 182 | 189 |
| | Window (in days) | | | | | | | | | |
| | (±3) | (±3) | (±3) | (±3) | (±3) | (±3) | (±3) | (±3) | (±3) | (±2) |
| Informed Consent/Assent | | | | | | | | | | |
| Eligibility Assessment | | | | | | | | | | |
| Demographics | | | | | | | | | | |
| Medical History | | | | | | | | | | |
| Physical & Vital signs^{c} | X | | X | | X | | X | | X | |
| ECG | X | | | | | | | | X | |
| Liver Ultrasound^{d} | | | | | | | | | X | |
| Pregnancy Test^{e} | U | | U | | U | | U | | S | |
| Confirm PFIC Genotype | | | | | | | | | | |
| Results^{f} | | | | | | | | | | |
| eDiary Device Provided/ | | | | | | | | | R | |
| Returned^{g} | | | | | | | | | | |
| ItchRO(Obs), ItchRO(Pt) & | Twice-daily completion of ItchRO & EDQ | | | | | | | | | |
| EDQ | | | | | | | | | | |
| Review eDiary & Assess | X | | X | | X | | X | | X | |
| Compliance | | | | | | | | | | |
| Randomized | | | | | | | | | | |
| CIS^{h} | X | | X | | X | | X | | X | |
| PISⁱ | X | | X | | X | | X | | X | |
| PIC | | | | | | | | | X | |
| CIC | | | | | | | | | X | |
| PedsQL^{j} | X | | X | | X | | X | | X | |
| Clinician Scratch Scale | X | | X | | X | | X | | X | |
| CBC with Differential | X | | X | | X | | X | | X | |
| Coagulation | X | | X | | X | | X | | X | |
| Chemistry Panel | X | | X | | X | | X | | X | |
| Lipid Panel^{k} | X | | X | | X | | X | | X | |
| Urinalysis | X | | | | X | | | | X | |
| FGF-19, Autotaxin, C4^{k} | X | | | | X | | | | X | |
| sBA collection^{k} | X | | X | | X | | X | | X | |
| Lipid Soluble Vitamins^{k,l} | X | | X | | X | | X | | X | |
| AFP Sample | X | | | | | | | | X | |
| PK Sample^{m} | X | | | | | | | | X | |
| Serum storage sample | X | | X | | X | | X | | X | |
| Healthcare Utilization | X | | X | | X | | X | | X | X |
| Study Medication | X | | X | | X | | X | | | |
| Suppliedⁿ | | | | | | | | | | |
| Study Medication | | | | | | | | | | |
| Administration^{o} | | | | | | | | | | |
| Assess AEs | X | X | X | X | X | X | X | X | X | X |
| Prior and Concomitant | X | X | X | X | X | X | X | X | X | X |
| Treatments | | | | | | | | | | |

AFP = a-fetoprotein;
C4 = 7a-hydroxy-4-cholesten-3-one;
CBC = complete blood count;
CIS = Caregiver Impression of Severity;
CIC = Caregiver Impression of Change;
ECG = electrocardiogram;
EDQ = exploratory diary questionnaire;
EOT = end of treatment;
ET = early termination;
FGF-19 = fibroblast growth factor-19;
ItchRO = Itch Reported Outcome;
P = Provided;
PedsQL = Pediatric Quality of Life Inventory;
PIC = Patient Impression of Change;
PIS = Patient Impression of Severity of Pruritus;
PK = pharmacokinetic;
R = Returned;
S = Serum;
U = Urine;
V = visit
^{a} Subjects who initially fail to meet eligibility criteria may be re-assessed during the 6-week screening period prior to being captured as a screen failure. Subjects may also be re-screened.
^{b}Study sites should record dates of any future scheduled procedures related to PFIC (e.g., PEBD, ileal exclusion, liver transplant, or listed for liver transplant), if known at this visit.
^{c}Blood pressure, heart rate, temperature, respiration rate. Height and weight will be measured by trained staff using standardized methodology, incl. calibrated stadiometer or headboard and calibrated balance, respectively.
^{d}Screening ultrasound not required if an ultrasound or liver MRI less than 6 months old is available.
^{e}Females of childbearing potential only; result must be reviewed prior to dispensing study medication.
^{f}Genotyping results will be reviewed by sponsor or designee.
^{g}Caregivers and age-appropriate subjects will be instructed to complete their eDiary twice daily (morning and evening).
^{h}To be completed by caregivers for all subjects
ⁱTo be completed only by subjects aged ≥9 years at screening
^{j}To be completed by subjects and caregivers using the age-appropriate PedsQL module
^{k}Subjects should make every effort to fast at least 6 hours prior to collection. Water intake is permitted if necessary but not recommended.
^{l}Blood samples must be drawn before administration of vitamin supplementation.
^{m}PK samples will be drawn pre-dose, and approximately 2.5 hours after the morning dose.
ⁿIf needed, study medication may be supplied via direct-to-patient shipments in between site visits.
^{o}Subjects will self-administer (or will be administered) the first dose of study medication in the clinic on Visit 1/Day 0 after breakfast under the supervision of the investigator or trained site staff

Serum bile acids and other cholestasis biomarkers. Blood samples were collected as described in Table 1 to measure levels of cholestasis biomarkers including total sBA, sBA subspecies, C4, FGF-19, and autotaxin as well as liver related parameters. Subjects were encouraged to fast at least 6 hours prior to collection (water intake was permitted if necessary but not recommended). Total sBA and targeted bile acid subspecies were quantified with liquid chromatography mass spectrometry (LC-MS) methodology for exploratory assessments. In addition, screening total sBA will be assessed with an enzymatic assay for inclusion criteria evaluation. C4, a key intermediate in the pathway for bile acid synthesis from cholesterol, will be determined by a validated LC-MS/MS method.

Clinical Laboratory Evaluations. Clinical laboratory assessments were performed as listed in Table 2. Serum bile acid results were blinded to sites and to the blinded study team until after database lock of study MRX-502 except for results at screening. Anion gap and osmolar gap as well as corrected sodium, α-Tocopherol/Total Lipids Ratio, Retinol/RBP Molar Ratio, FIB-4, and APRI were calculated.

Health-related Quality of Life and Pruritus Assessment. Patients were also administered a health-related quality of life (HRQoL) assessment throughout the study. The PedsQL™ is a questionnaire that was administered to subjects and caregivers using the age-appropriate PedsQL module. Subjects aged 8 to 12 years, and 13 to 18 years self-completed the PedsQL Child Report, and the PedsQL Teenager Report, respectively. Caregivers of subjects completed the age-appropriate Parent PedsQL report (i.e., Report for Infants, Toddlers, Young Children, Children, and Teenagers).

Caregiver Impression of Severity of Pruritus (CIS). The CIS is a questionnaire that was administered to caregivers as outlined in Table 2. The CIS is designed to assess the caregiver's perception of itch severity of their children. The questionnaire was administered with a recall period of 1 week.

Patient Impression of Severity of Pruritus (PIS). The PIS is a questionnaire that was administered to caregivers as outlined in Table 2. Subjects aged ≥9 years self-completed the questionnaire. The PIS is designed to assess the subject's perception of their itch severity. The questionnaire was administered with a recall period of 1 week.

Clinician Scratch Scale (CSS). The CSS provides an assessment of itch severity. The clinician's assessment of the subject's pruritus focused on scratching and visible damage to the skin as a result of scratching as observed by the physician. The CSS uses a 5-point scale, in which 0 designates no evidence of scratching and 4 designates cutaneous mutilation with bleeding, hemorrhage and scarring. A clinician's assessment of pruritus made by the principal investigator or sub-investigator using the CSS was recorded at screening, baseline, and at additional study visits as outline in Table 2.

TABLE 2
List of Laboratory Analytes

| | | | |
|---|---|---|---|
| Serum β-hCG | Clinical Chemistry | Lipid Panel^{a} | Urinalysis |
| (if indicated) | Albumin | Total cholesterol | pH |
| CBC with | ALP | LDL-C (direct) | Specific gravity |
| Differential | Amylase | HDL-C | Protein |
| Hematocrit | ALT (SGPT) | Triglycerides (TG) | Glucose |
| Hemoglobin | AST (SGOT) | Cholestasis | Ketones |
| MCV, MCH, | Bicarbonate | Biomarkers^{a} | Bilirubin |
| MCHC | Bilirubin, direct | Total serum bile acids | Occult blood and cells |
| Red blood cells | (conjugated) | (sBA) | Nitrite |
| Platelets | Total serum bilirubin | Serum bile acid | Urobilinogen |
| White blood cells | (TSB) | subspecies | Leukocyte esterase |
| WBC Differential | Blood urea nitrogen | 7α-hydroxy-4-cholesten- | Microscopic examination^{b} |
| (% and absolute) | (BUN) | 3-one (C4) | Oxalate |
| Neutrophils | Calcium | FGF-19 | Urinary creatinine |
| Eosinophils | Chloride | Autotaxin | Maralixibat |
| Basophils | Creatinine | Lipid Soluble | Levels |
| Lymphocytes | GGT | Vitamins^{a} | Maralixibat in plasma |
| Monocytes | Glucose | 25-hydroxy vitamin D | Marker of hepatocellular |
| Coagulation | Lipase | Retinol | carcinoma |
| aPTT (sec) | Phosphate | Retinol binding protein | α-Fetoprotein (AFP) |
| NR | Potassium | (RBP) | |
| PT (sec) | Sodium | α-Tocopherol | |
| | Total protein | | |
| | Uric Acid | | |
| | Measured serum | | |
| | Osmolality | | |

AFP = α-fetoprotein; ALP-alkaline phosphatase; ALT = alanine aminotransferase; aPTT = activated partial thromboplastin time; AST = aspartate aminotransferase; β-hCG = beta human chorionic gonadotropin; FGF-19 = fibroblast growth factor 19; FIB-4 = fibrosis-4; GGT = gamma-glutamyl transferase; PT = prothrombin time; MCH = mean corpuscular hemoglobin; MCHC = mean corpuscular hemoglobin concentration; MCV = mean corpuscular volume; RBP = Retinol binding protein: SGOT = serum glutamic-oxaloacetic transaminase; SGPT = serum glutamic-pyruvic transaminase; WBC = white blood cell.
^{a}Blood samples for the analysis of cholestasis biomarkers, lipid panel and lipid soluble vitamins should be drawn prior to administration of vitamin supplementation and as much as possible approximately 6 hours after food or formula (water intake is permitted if necessary but not recommended). Other biomarkers [e.g., lysophosphatidic acid (LPA),] may be measured. At the discretion of the sponsor, samples will be collected and appropriately stored for subsequent analysis, as needed.
^{b}Will be performed on abnormal findings unless otherwise specified.

Patient Impression of Change (PIC). The PIC is designed to assess the subject's perception of his/her itching at Week 26 (EOT) compared to his/her itching prior to the start of treatment with study drug. The PIC was completed by subjects who were 9 years of age or older at the Week 26 (EOT) visit.

Caregiver Impression of Change (CIC). The CIC is designed to assess the caregiver's perception of the subject's itch related symptoms and xanthoma severity at Week 26 (EOT) compared to his/her itch related symptoms and xanthoma severity prior to the start of treatment with study drug. The CIC was completed by all caregivers at the Week 26 (EOT) visit.

Exploratory Diary Questionnaire. Pruritus was assessed using the EDQ caregiver/patient reported outcome measure administered as a twice daily electronic diary. Caregivers for all subjects aged <9 years completed the Observer instrument: EDQ(Obs). Subjects ≥9 years of age completed the patient instrument: EDQ(Pt). Subjects and caregivers were trained on the use of the electronic diary during the screening visit (Visit 0). Pruritus was assessed and recorded twice daily by subjects or caregivers, via EDQ, beginning with the day after the screening visit and every day throughout the duration of the study, as described in Table 2.

Clinical Pharmacology Assessments. Blood samples (anticoagulant K3 EDTA) were drawn pre-dose and 2.5 hours (with a 30-minute window) post-morning dose at Week 10 (Visit 5) and Week 26 (Visit 9; EOT/ET) to assess the plasma level of maralixibat. Actual PK blood sample collection time versus time of dosing was recorded.

Healthcare Utilization. The number of hospitalizations related to underlying disease, length of stay for hospitalizations, and emergency room visits (days) and any surgeries and procedures specific to subject's PFIC condition were collected during post-baseline visits as outlined in Table 2. The date of visit/admission, date of discharge, relationship/issue, and discharge status were collected.

### PFIC Populations for Analyses

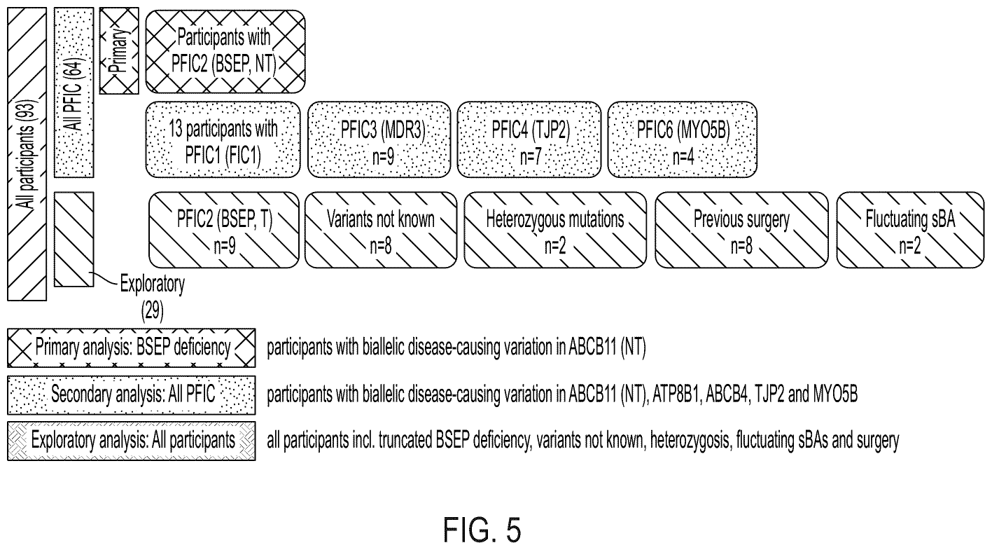

The following definitions are used for the PFIC populations in the analyses of the various endpoints in this study, as summarized in FIG. 5. Primary and secondary endpoints: 1) PFIC 2, participants with PFIC 2 who fulfill criteria for the primary cohort; 2) PFIC, participants with PFIC 1, PFIC 2 (who fulfill criteria for the primary cohort), PFIC 3, PFIC 4, PFIC 5, and PFIC 6. Exploratory Endpoints: PFIC 2 and PFIC 1, PFIC 2, PFIC 4, PFIC 5, PFIC 6, truncated PFIC 2.

In the PFIC population (PFIC 1, PFIC 2, PFIC 3, PFIC 4, PFIC 5, and PFIC 6), participants with biallelic disease causing variation in ATP8B1 (PFIC 1), ABCB11 (PFIC 2, truncated and non-truncated variants), ABCB4 (PFIC 3), TJP2 (PFIC 4), MYO5B (PFIC 6), as well as PFIC patients with a history of surgical biliary diversion or Kasai, PFIC 1 and PFIC 2 patients with heterozygosis and participants with an unknown variant were included. The number of participants with each variation was dependent on the genotype prevalence within the enrolled study population, see FIG. 5 and Tables 3 and 4.

Percentages are 100*n/N, unless otherwise noted. Subjects with PFIC 1 through PFIC 6 all have the biallelic disease. [1] nt-PFIC 2=non-truncated PFIC 2 (primary cohort); nt-PFIC 2-IsBA=non-truncated PFIC 2 with low or fluctuating serum bile acids; nt-PFIC 2-surg=non-truncated PFIC 2 with a history of surgery; t-PFIC 2=truncated PFIC 2; PFIC 1-surg=PFIC 1 with a history of surgery; PFIC 4-surg=PFIC 4 with a history of surgery; nt PFIC 2-het=non-truncated PFIC 2 with heterozygosis; PFIC 1-het=PFIC 1 with heterozygosis; No-variant-found =No established variant linked to PFIC disease. [2] nt PFIC 2=partial loss of B SEP function; t-PFIC 2=complete loss of BSEP function. Only applicable for PFIC 2 subjects, with exception of one nt PFIC 2 subject (015002) with heterozygosis ABCB11 mutation; N/A for all other subjects. [3] One subject (015002) had heterozygous ABCB11 mutation, and another (021006) had heterozygous ATP8B1 mutation.

Table 3 provides a summary of disposition and demographics for participants in the MARCH clinical study. Table 4 provides a summary of baseline health-related parameters for participants in the MARCH clinical study. Described table results of Tables 3 and 4 include subject disposition, demographics and baseline characteristics, PFIC disease history, prior medications, and treatment exposure and compliance. Table 3 shows that baseline characteristics and demographics were balanced between the cohorts.

TABLE 3
Demographics for participants in the clinical study

| | Primary Cohort | | | | | | PFIC Cohort | | | |
|---|---|---|---|---|---|---|---|---|---|---|
| Status o [symbol] | Mara [symbol] | | Placebo | | Overall | | Mara [symbol] | | Placebo | |
| Category | (N = 14) | | (N = 17) | | (N = 31) | | (N = 33) | | (N = 31) | |
| Screened for Eligibility | | | | | | | | | | |
| Screen Failure | | | | | | | | | | |
| Randomized | 14 | | 17 | | 31 | | 33 | | 31 | |
| Total Number of Siblings | 2 | | 0 | | 2 | | 4 | | 4 | |
| Safety Population | [symbol] 4 | | 17 | | [symbol] 1 | | [symbol] | | 31 | |
| ITT Population | [symbol] 4 | (100.0%) | 17 | (100.0%) | 31 | (100.0%) | 3 [symbol] | (100.0%) | 31 | (100.0%) |
| Per-Protocol Population | 9 | (64.3%) | 17 | (100.0%) | 2 [symbol] | (83.9%) | 27 | (81. [symbol] %) | 31 | ( [symbol] 00.0%) |
| Completed Study Treatment | 1 [symbol] | (92.9%) | 15 | ( [symbol] 8.2%) | 2 [symbol] | ( [symbol] 0.3%) | 32 | (97.0%) | 28 | ( [symbol] .3%) |
| Discontinued Early | 1 | (7. [symbol] %) | 2 | (1 [symbol] .8%) | 3 | (9.7%) | 1 | (3.0%) | 3 | (9.7%) |
| Reason for Discontinuation | | | | | | | | | | |
| Adverse event | 0 | | 0 | | 0 | | 0 | | 0 | |
| Death | 0 | | 0 | | 0 | | 0 | | 0 | |
| Lost to follow-up | 0 | | 0 | | 0 | | 0 | | 0 | |
| Non-compliance with study drug | 0 | | 0 | | 0 | | 0 | | 0 | |
| Lack of efficacy | 0 | | 0 | | 0 | | 0 | | 0 | |
| Physician decision | 0 | | 0 | | 0 | | 0 | | 0 | |
| Pregnancy | 0 | | 0 | | 0 | | 0 | | 0 | |
| Liver transplant | 0 | | 0 | | 0 | | 0 | | 0 | |
| PE [symbol] D surgery | 0 | | 0 | | 0 | | 0 | | 0 | |
| Protocol violation | 0 | | 0 | | 0 | | 0 | | 0 | |
| Withdrawal of consent | 1 | (7.1%) | [symbol] | (5. [symbol] %) | 2 | ( [symbol] .5%) | [symbol] | (3.0%) | 2 | ( [symbol] .5%) |

| Study terminated by sponsor | 0 | 0 | | 0 | | 0 | 0 | |
|---|---|---|---|---|---|---|---|---|
| Disease progression | 0 | 1 | (5.9%) | [symbol] | (3.2%) | 0 | [symbol] | (3.2%) |

| | | | | | |
|---|---|---|---|---|---|
| Other | 0 | 0 | 0 | 0 | 0 |

| | PFIC Cohort | | All subjects | | | | | |
|---|---|---|---|---|---|---|---|---|
| Status of | Overall | | Mara [symbol] | | Placebo | | Overall | |
| Category | (N = 64) | | (N = 47) | | (N = 46) | | (N = 125) | |
| Screened for Eligibility | | | | | | | 125 | |
| Screen Failure | | | | | | | [symbol] 2 | |
| Randomized | 64 | | 47 | | 46 | | [symbol] 3 | |
| Total Number of Siblings | [symbol] | | 4 | | 6 | | 10 | |
| Safety Population | 64 | | 47 | | 4 [symbol] | | [symbol] 3 | |
| ITT Population | [symbol] 4 | (100.0%) | 47 | (100.0%) | 4 [symbol] | (100.0%) | [symbol] 3 | (100.00%) |
| Per-Protocol Population | [symbol] | (90.6%) | 41 | (87.2%) | 45 | (97.8%) | 86 | (92.5%) |
| Completed Study Treatment | 60 | (93.8%) | 44 | (9 [symbol] . [symbol] %) | 42 | (91.3%) | [symbol] | (92.5%) |
| Discontinued Early | 4 | (6.3%) | [symbol] | (6.4%) | 4 | ( [symbol] .7%) | 7 | (7.5%) |
| Reason for Discontinuation | | | | | | | | |
| Adverse event | 0 | | 1 | (2.1%) | 0 | | 1 | (1.1%) |
| Death | 0 | | 0 | | 0 | | 0 | |
| Lost to follow-up | 0 | | 0 | | 0 | | 0 | |
| Non-compliance with study drug | 0 | | 0 | | 0 | | 0 | |
| Lack of efficacy | 0 | | 0 | | 0 | | 0 | |
| Physician decision | 0 | | 0 | | 0 | | 0 | |
| Pregnancy | 0 | | 0 | | 0 | | 0 | |
| Liver transplant | 0 | | 1 | (2.1%) | 0 | | 1 | (1.1%) |
| PE [symbol] D surgery | 0 | | 0 | | 0 | | 0 | |
| Protocol violation | 0 | | 0 | | 0 | | 0 | |
| Withdrawal of consent | 3 | (4.7%) | 1 | (2.1%) | 3 | (6.5%) | 4 | (4.3%) |

| Study terminated by sponsor | 0 | | 0 | 0 | | 0 | |
|---|---|---|---|---|---|---|---|
| Disease progression | 1 | ( [symbol] .6%) | 0 | 1 | (2.2%) | 1 | (1.1%) |

| | | | | |
|---|---|---|---|---|
| Other | 0 | 0 | 0 | 0 |

[1] Age at baseline visit.
[2] Height, weight, and BMI Z-scores are based on a subject's sex and age at the baseline visit.
The World Health Organization (WHO) growth charts were used to derive Z-scores for subjects less than 24 months old and the Center for Disease Control (CDC) growth charts were used to derive Z-scores for subjects equal to or greater than 24 months old. [symbol]
indicates data missing or illegible when filed

TABLE 4
Baseline disease characteristics for participants in the clinical study

| | | Primary Cohort | | | | | | PFIC Cohort | | | |
|---|---|---|---|---|---|---|---|---|---|---|---|
| | Statistic | Maralixibat | | Placebo | | Overall | | Maralixibat | | Placebo | |
| Variable | or Category | (N = 14) | | (N = 17) | | (N = 31) | | (N = 33) | | (N = 31) | |
| Clinician Scratch | Mean | 2.8 | | 2.5 | | 2.6 | | 2.8 | | 2.6 | |
| Scale (CSS) Score | 0 | 0 | | 0 | | 0 | | 0 | | 0 | |
| | 1 | 0 | | 3 | (17.6%) | 3 | (9.71%) | 1 | (3.0%) | 6 | (19.4%) |
| | 2 | 6 | (42.9%) | 4 | (23.5%) | 10 | (32.3%) | 10 | (30.3%) | 5 | (16.1%) |
| | 3 | 5 | (35.7%) | 8 | (47.1%) | 13 | (41.9%) | 17 | (51.5%) | 15 | (48.4%) |
| | 4 | 3 | (21.4%) | 2 | (11.8%) | 5 | (16.1%) | 5 | (15.2%) | 5 | (16.1%) |
| ItchRO (Obs) 4-wk Morning | Mean | 2.876 | | 2.611 | | 2.731 | | 2.850 | | 2.732 | |
| Avg Severity Score | | | | | | | | | | | |
| ItchRO (Obs) 4-wk Evening | Mean | 2.823 | | 2.554 | | 2.676 | | 2.794 | | 2.657 | |
| Avg Severity Score | | | | | | | | | | | |
| ItchRO (Obs) 4-wk Highest | Mean | 3.029 | | 2.796 | | 2.901 | | 3.010 | | 2.877 | |
| Daily Avg Severity Score | | | | | | | | | | | |
| Time Since Original Diagnosis | Mean | 45.9 | | 33.5 | | 39.1 | | 34.3 | | 27.5 | |
| of PFIC (months) | | | | | | | | | | | |
| Baseline UDCA Usage | Mean | 11 | (78.6%) | 17 | (100.0%) | 28 | (90.3%) | 27 | (81.8%) | 30 | (96.8%) |
| Baseline Rifampicin Usage | Mean | 6 | (42.9%) | 9 | (52.9%) | 15 | (48.4%) | 18 | (54.5%) | 15 | (48.4%) |

| | | | | | | |
|---|---|---|---|---|---|---|
| Total Serum Bile Acid (umol/L) | Mean | 331.879 | 312.278 | 312.113 | 254.327 | 272.297 |
| Alkaline Phosphatase (U/L) | Mean | 535.75 | 442.89 | 484.83 | 630.14 | 517.73 |
| Aspartate Aminotransferase (U/L) | Mean | 103.75 | 158.07 | 133.54 | 96.89 | 129.81 |
| Alanine Aminotransferase (U/L) | Mean | 98.39 | 154.91 | 129.39 | 87.83 | 127.34 |
| Total Bilirubin (mg/dL) | Mean | 3.475 | 2.714 | 3.058 | 4.118 | 4.041 |
| Direct Bilirubin (mg/dL) | Mean | 2.421 | 1.919 | 2.146 | 2.979 | 2.934 |
| GGT (U/L) | Mean | 18.43 | 22.28 | 20.54 | 64.91 | 70.33 |
| Albumin (g/dL) | Mean | 4.561 | 4.875 | 4.733 | 4.573 | 4.717 |

| | | PFIC Cohort | | All Subjects | | | | | |
|---|---|---|---|---|---|---|---|---|---|
| | Statistic or | Overall | | Maralixibat | | Placebo | | Overall | |
| Variable | Category | (N = 64) | | (N = 47) | | (N = 46) | | (N = 93) | |
| Clinician Scratch | Mean | 2.7 | | 2.7 | | 2.7 | | 2.7 | |
| Scale (CSS) Score | 0 | 0 | | 1 | (2.1%) | 0 | | 1 | (1.1%) |
| | 1 | 7 | (10.9%) | 1 | (2.1%) | 6 | (13.0%) | 7 | (7.5%) |
| | 2 | 15 | (23.4%) | 15 | (31.9%) | 10 | (21.7%) | 25 | (26.9%) |
| | 3 | 32 | (50.0%) | 22 | (46.8%) | 20 | (43.5%) | 42 | (45.2%) |
| | 4 | 10 | (15.6%) | 8 | (17.0%) | 10 | (21.7%) | 18 | (19.4%) |
| ItchRO (Obs) 4-wk Morning | Mean | 2.793 | | 2.842 | | 2.921 | | 2.881 | |
| Avg Severity Score | | | | | | | | | |
| ItchRO (Obs) 4-wk Evening | Mean | 2.728 | | 2.786 | | 2.812 | | 2.799 | |
| Avg Severity Score | | | | | | | | | |
| ItchRO (Obs) 4-wk Highest | Mean | 2.945 | | 2.995 | | 3.051 | | 3.023 | |
| Daily Avg Severity Score | | | | | | | | | |
| Time Since Original Diagnosis | Mean | 31.0 | | 31.7 | | 32.7 | | 32.2 | |
| of PFIC (months) | | | | | | | | | |
| Baseline UDCA Usage | Mean | 57 | (89.1%) | 39 | (83.0%) | 39 | (84.8%) | 78 | (83.9%) |
| Baseline Rifampicin Usage | Mean | 33 | (51.6%) | 26 | (55.3%) | 23 | (50.0%) | 49 | (52.7%) |

| | | | | | |
|---|---|---|---|---|---|
| Total Serum Bile Acid (umol/L) | Mean | 263.312 | 262.615 | 243.306 | 252.854 |
| Alkaline Phosphatase (U/L) | Mean | 575.69 | 594.87 | 512.85 | 554.30 |
| Aspartate Aminotransferase (U/L) | Mean | 112.84 | 119.65 | 125.58 | 122.58 |
| Alanine Aminotransferase (U/L) | Mean | 106.97 | 107.86 | 121.23 | 114.47 |
| Total Bilirubin (mg/dL) | Mean | 4.081 | 4.099 | 3.796 | 3.950 |
| Direct Bilirubin (mg/dL) | Mean | 2.957 | 2.989 | 2.769 | 2.880 |
| GGT (U/L) | Mean | 67.54 | 51.56 | 59.45 | 55.46 |
| Albumin (g/dL) | Mean | 4.643 | 4.667 | 4.686 | 4.676 |

TABLE 5
Baseline Characteristics and Demographics Between the Cohorts

| | BSEEP | | All-PFIC | | Full Study | |
|---|---|---|---|---|---|---|
| | Maralixibat | Placebo | Maralixibat | Placebo | Maralixibat | Placebo |
| Variable | (n = 14) | (n = 17) | (n = 33) | (n = 31) | (n = 47) | (n = 46) |
| Age (years); mean | 6.3 | 4.2 | 4.9 | 4.4 | 4.8 | 4.7 |
| Sex (male); % | 50 | 35 | 52 | 42 | 43 | 48 |
| Pruritus (ItchRO[Obs]); mean | 2.9 | 2.6 | 2.9 | 2.7 | 2.8 | 2.9 |
| Total sBA (\|imol/L); mean | 312 | 312 | 254 | 272 | 263 | 243 |
| UDCA usage (%) | 79 | 100 | 82 | 97 | 83 | 85 |
| Rifampicin usage (%) | 43 | 53 | 55 | 48 | 55 | 50 |
| Alanine aminotransferase (U/L); mean | 98.4 | 154.9 | 87.8 | 127.3 | 107.9 | 121.2 |
| Total bilirubin (mg/dL); mean | 3.5 | 2.7 | 4.1 | 4.0 | 4.1 | 3.8 |
| Direct bilirubin (mg/dL); mean | 2.4 | 1.9 | 3.0 | 2.9 | 3.0 | 2.8 |
| Height Z-score; mean | −2.0 | −2.2 | −2.1 | −2.1 | −2.0 | −1.9 |
| Weight Z-score; mean | −1.5 | −1.2 | −1.8 | −1.3 | −1.6 | −1.2 |

### Example 2. Primary Efficacy Endpoint

For this study, the primary estimand is the improvement in pruritus measured as change from baseline in the average morning ItchRO(Obs) severity score in the maralixibat treatment group relative to the placebo group. The primary analysis on the primary efficacy endpoint was conducted in 1) participants with PFIC 2 who fulfill criteria for the primary cohort in the ITT population (non-truncated primary “B SEP cohort” or “Primary cohort” with N=31), 2) all PFIC participants (excluding the participants with prior history of surgery, heterozygous participants and unknown types, “All PFIC cohort” or “PFIC cohort” with N=64), and 3) all study participants cohort, including the participants with prior history of surgery, heterozygous participants and unknown types. A restricted maximum likelihood (REML)-based mixed-effects model for repeated measures (HHRH) was used as the primary analysis method. The repeated measures includes post-baseline visits during the dose escalation phase (i.e., Week 6) and stable dosing phase (i.e., Weeks 10, 14, 18, 22, and 26), with change from baseline in the 6- or 4-week average morning ItchRO(Obs) severity score as the dependent variable. The MMRM model includes the fixed, categorical effects of treatment group, visit, and treatment group-by-visit interaction as well as the continuous, fixed covariates of baseline 4-week average morning ItchRO(Obs) severity score and the baseline score-by-visit interaction.

The primary efficacy analysis compared maralixibat and placebo using the contrast (difference in least squares [LS] means) between treatment groups across the last 12 weeks of the study (i.e., Weeks 15-18, 19-22, and 23-26 combined). The analytical solution of the overall treatment effect obtained from MMRM is an equally weighted average of the 3 individual period-specific estimates over the time period of interest (i.e., the last 12 weeks of the study). Significance tests were based on LS means using a 2-sided significance level (2-sided 95% confidence intervals [CIs]). The null hypothesis for the primary efficacy endpoint of the equality of maralixibat and placebo is: H₀₁: mean change in average morning ItchRO(Obs) severity score between baseline and Week 15 through Week 26 in the 2 treatment groups are equal.

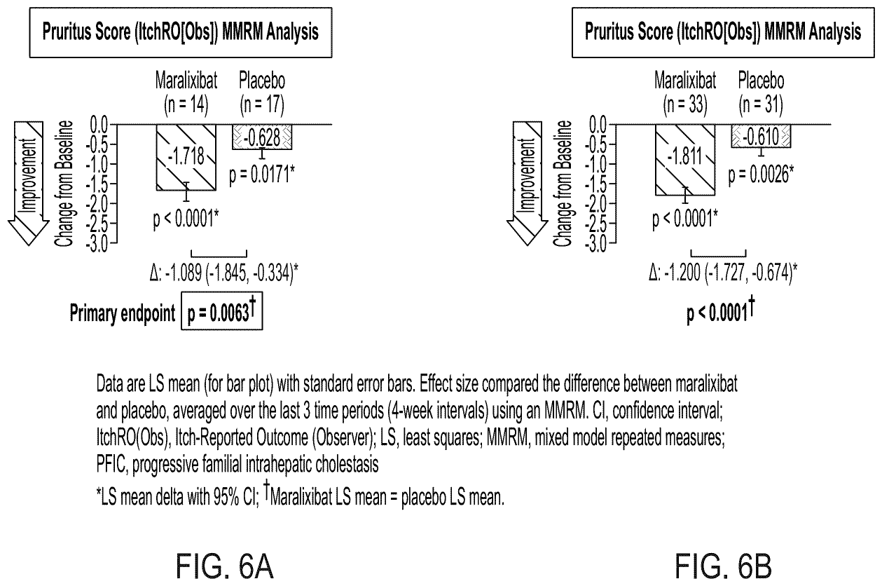

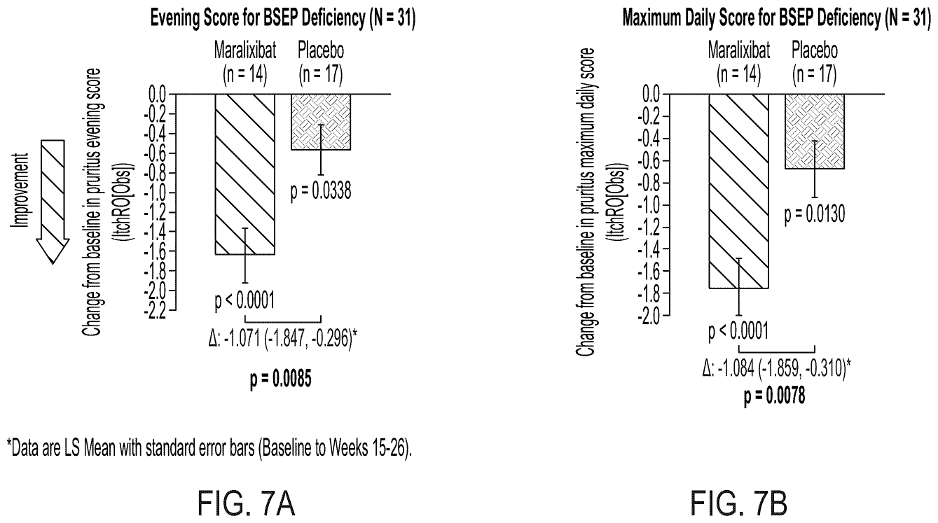

The primary efficacy endpoint (as mean change from baseline in pruritus severity score (ItchRO[Obs])) in BSEP deficiency cohort and All PFIC cohort are summarized in FIGS. 6-8. The BSEP deficiency cohort showed a reduction of 1.7 points from baseline in pruritus morning ItchRO[Obs] score, a 1.089 point difference from the placebo group (FIG. 6A). The BSEP deficiency cohort showed a reduction of 1.7 points from baseline in pruritus evening ItchRO[Obs] score, a 1.111 point difference from the placebo group (FIG. 7A). The BSEP deficiency cohort showed a reduction of 1.8 points from baseline in maximum pruritus daily ItchRO[Obs] score, a 1.130 point difference from the placebo group (FIG. 7B).

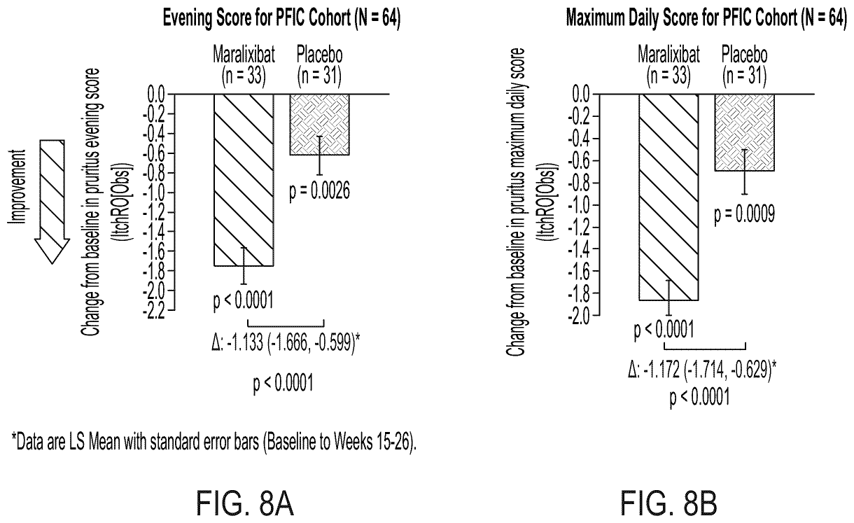

The All PFIC cohort showed a reduction of 1.8 points from baseline in pruritus morning ItchRO[Obs] score, a 1.20 point difference from the placebo group (FIG. 6B). The All PFIC cohort showed a reduction of 1.8 points from baseline in pruritus evening ItchRO[Obs] score, a 1.157 point difference from the placebo group (FIG. 8A). The All PFIC cohort showed a reduction of 1.9 points from baseline in maximum pruritus daily ItchRO[Obs] score, a 1.198 point difference from the placebo group (FIG. 8B).

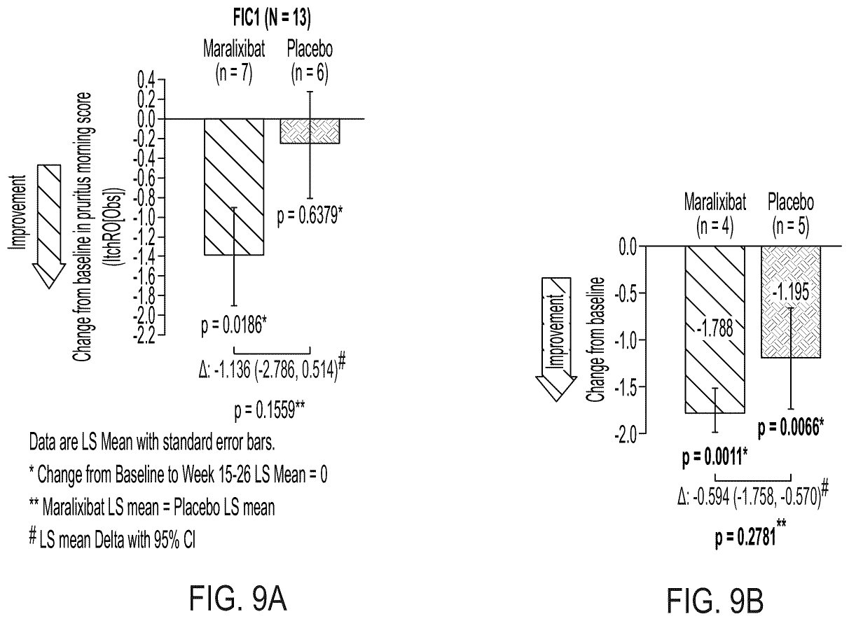

The primary efficacy endpoint (as mean change from baseline in pruritus severity score (ItchRO[Obs])) in FIC1 (PFIC 1) cohort and MDR3 (PFIC 3) cohort are summarized in FIG. 9. The FIC1 cohort showed a reduction of 1.4 points from baseline in pruritus morning ItchRO[Obs] score, a 1.136 point difference from the placebo group (FIG. 9A). The MDR3 cohort showed a reduction of 1.8 points from baseline in pruritus morning ItchRO[Obs] score, a 0.594 point difference from the placebo group (FIG. 9B).

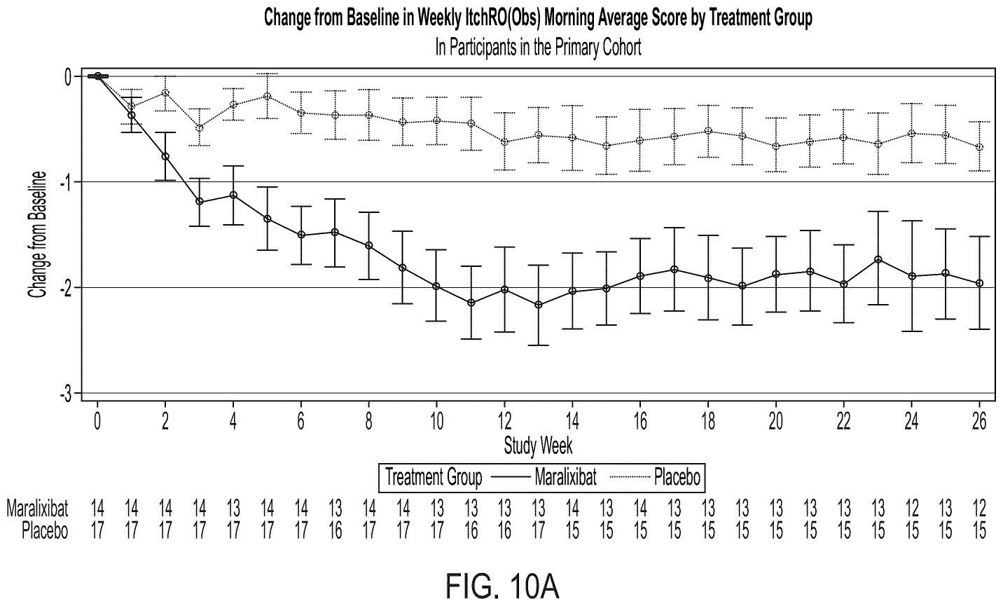

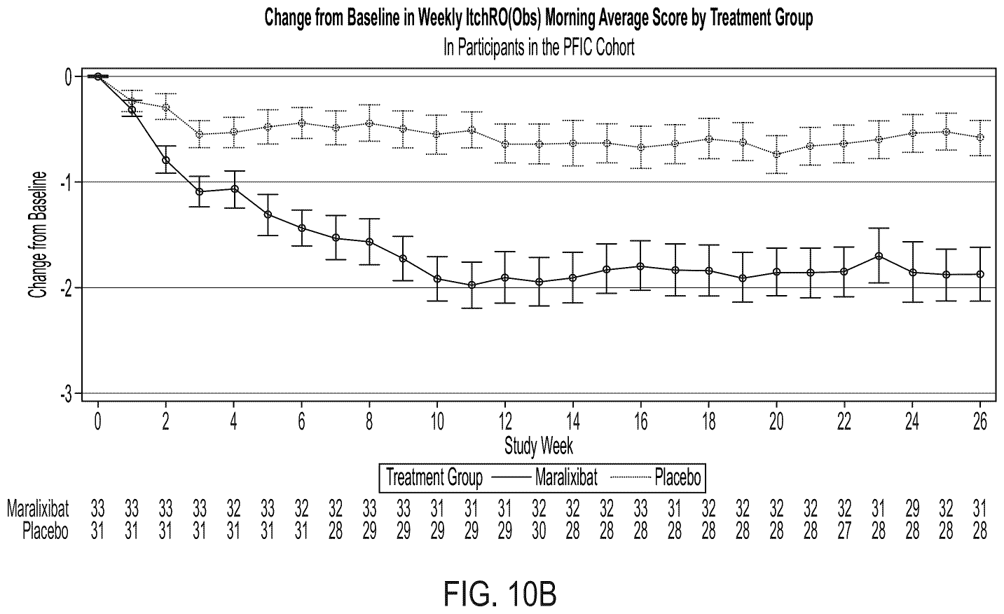

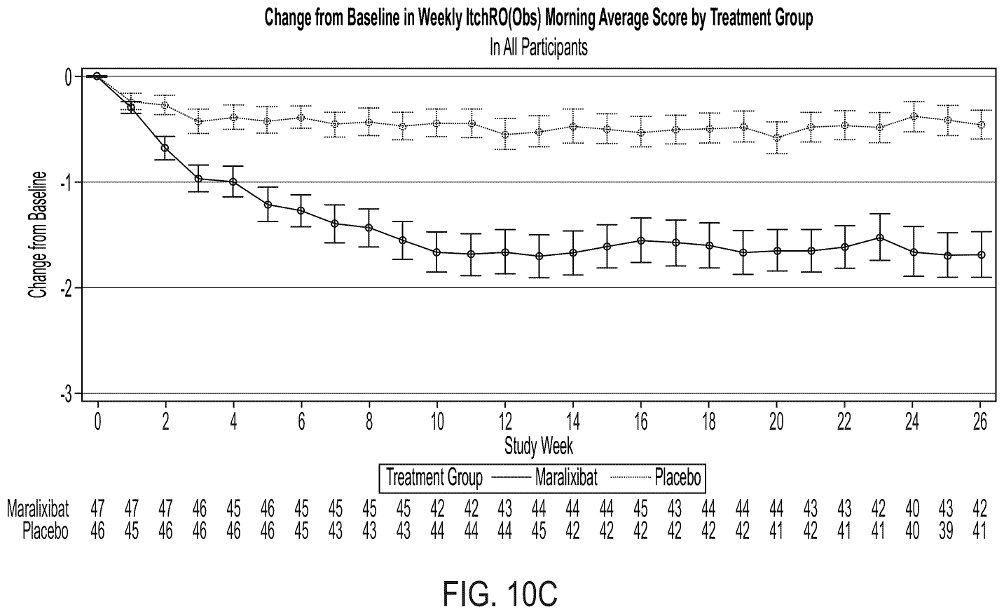

The change from baseline in weekly morning average ItchRO[Obs] score over time for the BSEP Deficiency (aka Primary) group is shown in FIG. 10A. The change from baseline in weekly morning average ItchRO[Obs] score over time for the PFIC group is shown in FIG. 10B. The change from baseline in weekly morning average ItchRO[Obs] score over time for all study participants is shown in FIG. 10C. It is clear from these figures that ItchRO[Obs] score for the maralixibat-treated participants is significantly lower at each time point from Week 2 to Week 26, indicating a very strong response to maralixibat treatment across all PFIC types.

These results demonstrate that maralixibat is highly effective as compared to placebo to significantly reduce pruritus in all PFIC patients, including PFIC 1 participants, PFIC 2 participants, PFIC 3 participants and all PFIC groups taken together.

### Example 3. Secondary Efficacy Endpoints

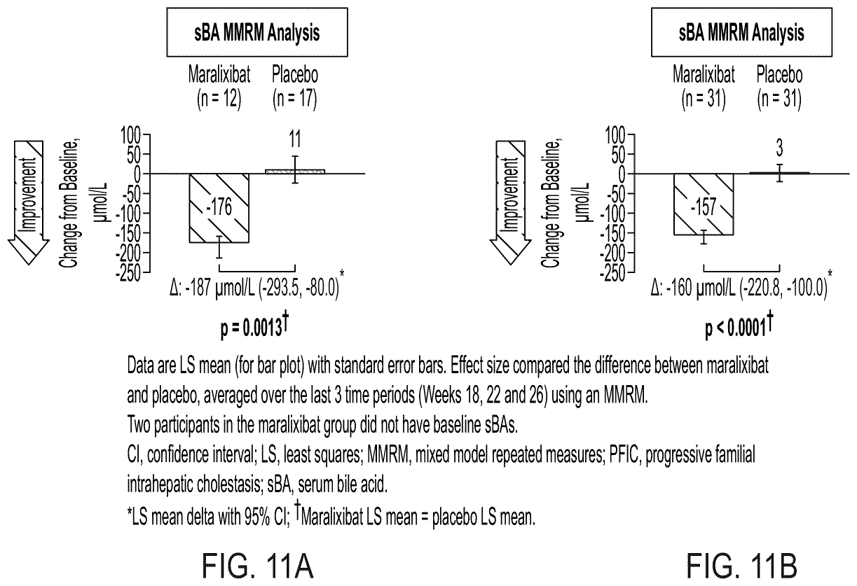

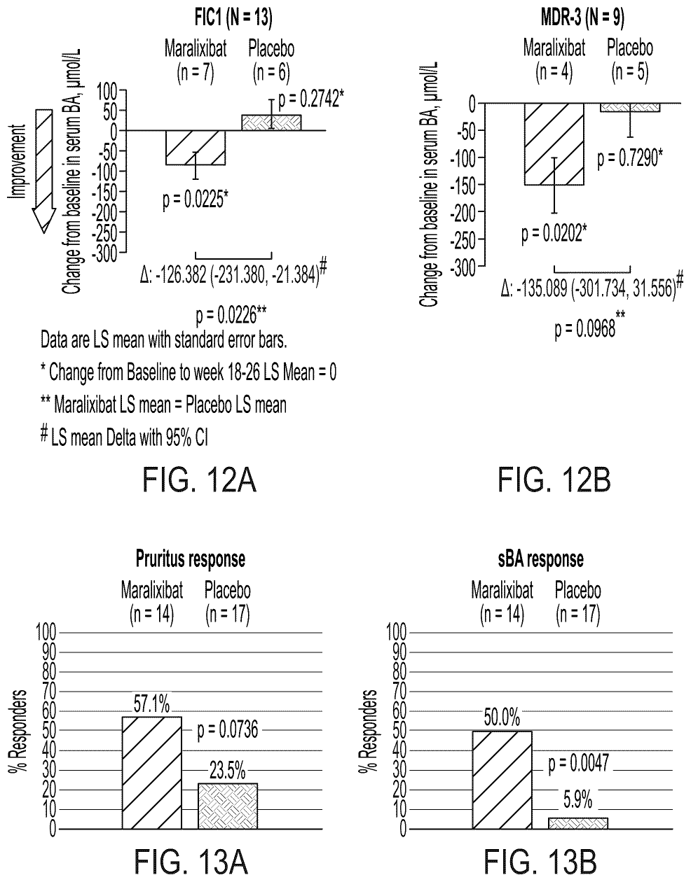

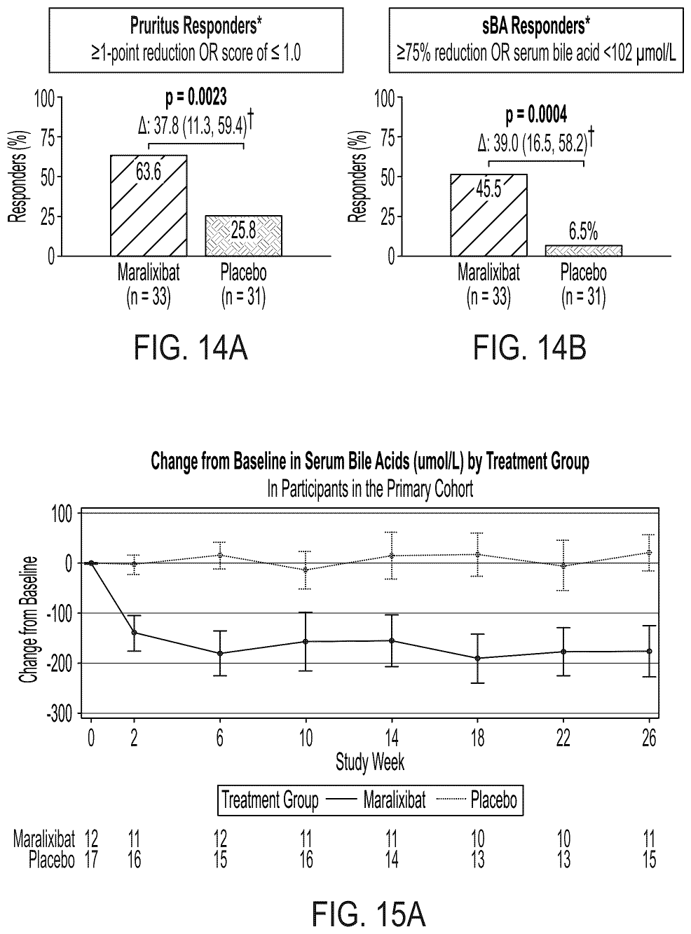

The secondary efficacy endpoints are defined as Mean change in total serum bile acid (sBA) level between baseline and average of Weeks 18, 22, and 26 (FIGS. 11-12); 2) Percentage of ItchRO(Obs) responders from Week 15 to Week 26 (FIGS. 13A and 14A); 3) Percentage of sBA responders from Week 18 to Week 26 (FIGS. 13B and 14B). Pruritus responders are defined as a subject having a 4-week average morning ItchRO (Obs) severity change from baseline of ≤1.0 OR an average severity score of ≤1.0. For the purpose of determining response, the average severity score from the three 4-week periods (weeks 15-18, 19-22 and 23-26) are used. A subject is defined as an ItchRO non-responder if the 4-week average baseline score is missing OR all three 4-week average (post-baseline) scores are missing. sBA responders are defined as a subject having an average sBA level of <102 μmon (applies only if baseline sBA was ≥102 μmon) OR a ≤−75% average percent change from baseline. For the purpose of determining response, the average sBA value from Weeks 18, 22 and 26 values are used. A subject is defined as an sBA non-responder if the baseline sBA value is missing OR sBA values are missing at all 3 time points (i.e., Weeks 18, 22, and 26). p-values comparing maralixibat to placebo treatment groups are calculated using Barnard's exact test.

The key secondary efficacy endpoint (as mean change from baseline in sBA levels) is shown in FIGS. 11-12. The BSEP deficiency cohort achieved a reduction of sBA levels of nearly 200 μmol/L, which is 186.723 μmol/L difference from placebo (FIG. 11A). The All PFIC cohort achieved a reduction of sBA levels of over 150 μmon, which is 160.403 μmon difference from placebo (FIG. 11B). The FIC1 cohort achieved a reduction of sBA levels of nearly 100 μmol/L, which is 126.382 μmol/L difference from placebo (FIG. 12A). The MDR3 cohort achieved a reduction of sBA levels of 150 μmon, which is 135.089 μmon difference from placebo (FIG. 12B).

Percentages of pruritus and sBA response for the BSEP deficiency cohort are shown in FIG. 13. The percentage of pruritus response in B SEP deficiency cohort taking maralixibat was 57.1%, vs 23.5% of placebo group, which represents a p value of 0.0736 (FIG. 13A). The percentage of sBA response in BSEP deficiency cohort taking maralixibat was 35.7%, vs 5.9% of placebo group, which represents a p value of 0.0410 (FIG. 13B).

Percentages of pruritus and sBA response for the All PFIC cohort are shown in FIG. 14. The percentage of pruritus response in All PFIC cohort taking maralixibat was 63.6%, vs 25.8% of placebo group, which represents a p value of 0.0023 (FIG. 14A). The percentage of sBA response in BSEP deficiency cohort taking maralixibat was 45.5%, vs 6.5% of placebo group, which represents a p value of 0.0004 (FIG. 14B).

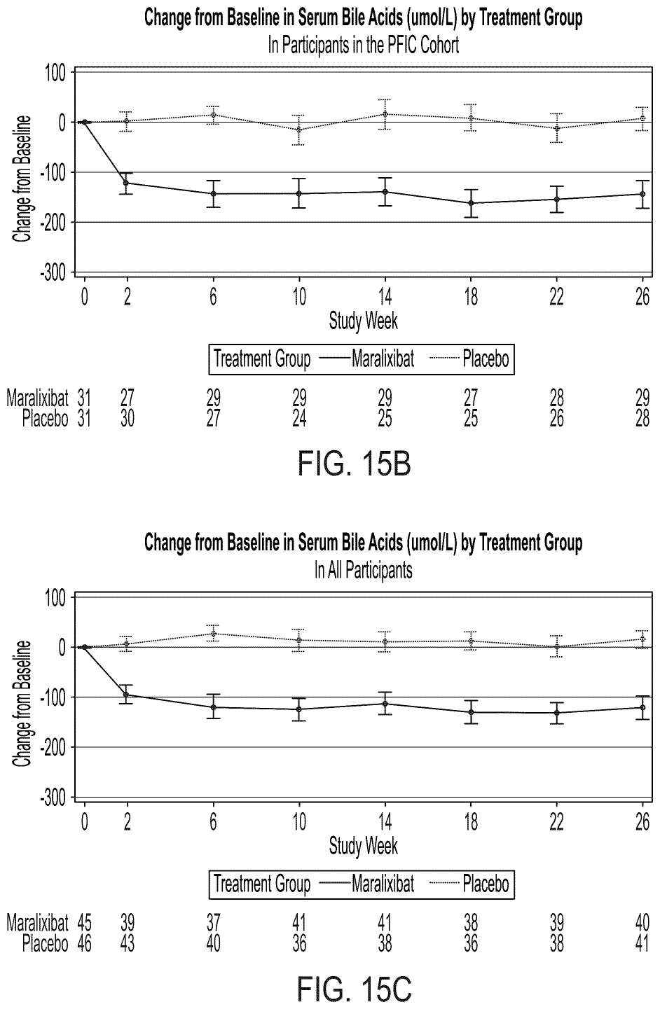

The change from baseline in sBA levels (μmol/L) over time for the BSEP Deficiency (aka Primary) group is shown in FIG. 15A. The change from baseline in sBA levels (μmol/L) over time for the PFIC group is shown in FIG. 15B. The change from baseline in sBA levels (μmol/L) over time for all study participants is shown in FIG. 15C. It is clear from these figures that sBA levels for the maralixibat-treated participants are more than 100 μmon lower at each time point from Week 2 to Week 26, indicating a very strong response to maralixibat treatment across all PFIC types.

There results demonstrate that maralixibat is highly effective in significantly reducing serum bile acids in all PFIC patients, including PFIC 1 patients, PFIC 2 patients, PFIC 3 patients and all PFIC groups taken together. The results further demonstrate that maralixibat is effective to reduce both pruritus and sBA in the majority of participants, at a much higher fraction of participants than placebo, with a very high confidence degree.

### Example 4. Additional Efficacy Assessments

The additional efficacy endpoints are defined as: 1) pruritus response: proportion of assessments ≤1; 2) pruritus response: proportion assessments ≤1 or decrease ≥1; 3) mean change from baseline in Clinician Scratch Scale (CSS) score; 4) mean change from baseline in total bilirubin; and 5) mean change from baseline in direct bilirubin.

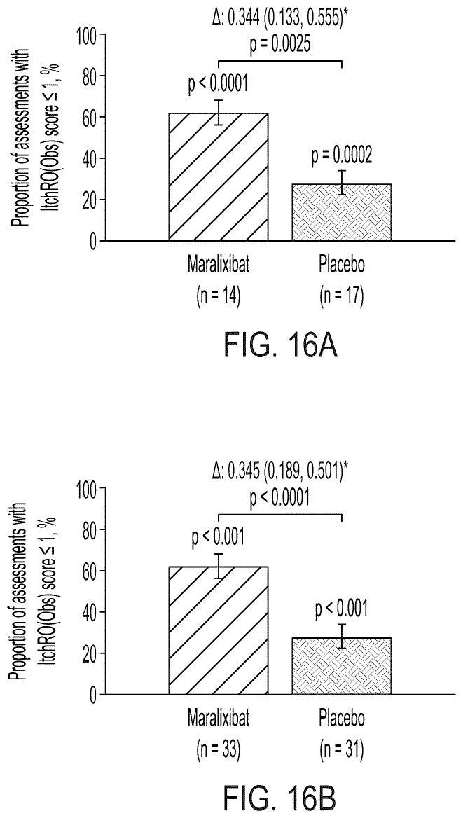

The pruritus response according to proportion of assessments ≤1 for BSEP deficiency cohort and PFIC cohort is shown in FIG. 16. A responder is defined as a scratching score of ≤=1 on the ItchRO(Obs) severity score. Morning and evening were used in the calculation of the proportion. [1] Model includes treatment arm, baseline morning pruritus score and baseline evening pruritus score. [2] Model includes treatment arm, baseline morning pruritus score, baseline evening pruritus score and PFIC type. The proportion of pruritus responders in the BSEP deficiency cohort participants taking maralixibat was over 0.6 vs. 0.3 for the placebo group, indicating a difference of 0.344 in proportion of pruritus responders taking maralixibat (FIG. 16A). The proportion of pruritus responders in the PFIC cohort participants taking maralixibat was over 0.6 vs. 0.3 for the placebo group, indicating a difference of 0.345 in proportion of pruritus responders taking maralixibat (FIG. 16B).

The pruritus response according to proportion of assessments ≤1 or decrease ≥1 for BSEP deficiency cohort and PFIC cohort is shown in FIG. 17. A responder is defined as a scratching score of <=1 or at least a one-point drop from baseline on the ItchRO(Obs) severity score. At each assessment, the morning severity score was compared to the baseline morning average severity score, and the evening severity score was compared to the baseline evening severity score average. Morning and evening were used in the calculation of the proportion. [1] Model includes treatment arm, baseline morning pruritus score and baseline evening pruritus score. [2] Model includes treatment arm, baseline morning pruritus score, baseline evening pruritus score and PFIC type. The proportion of pruritus responders in the B SEP deficiency cohort participants taking maralixibat was over 0.7 vs. 0.3 for the placebo group, indicating a difference of 0.398 in proportion of pruritus responders taking maralixibat (FIG. 17A). The proportion of pruritus responders in the PFIC cohort participants taking maralixibat was over 0.7 vs. 0.3 for the placebo group, indicating a difference of 0.380 in proportion of pruritus responders taking maralixibat (FIG. 17B).

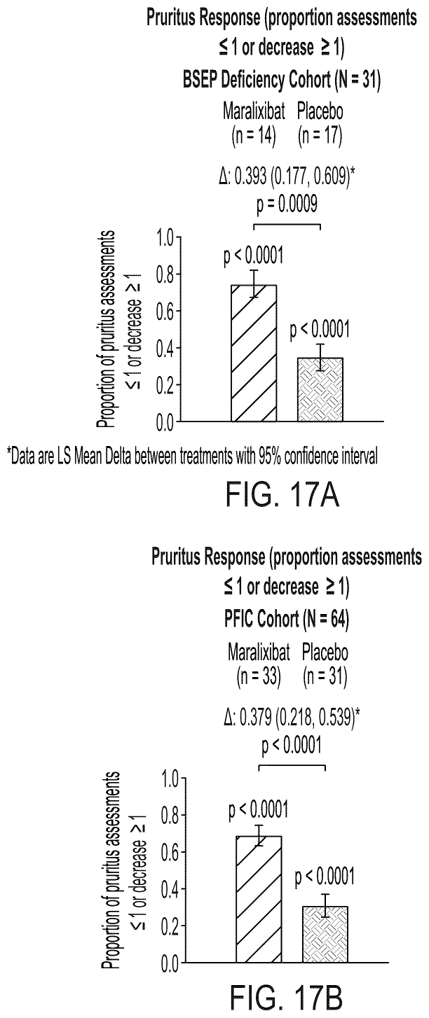

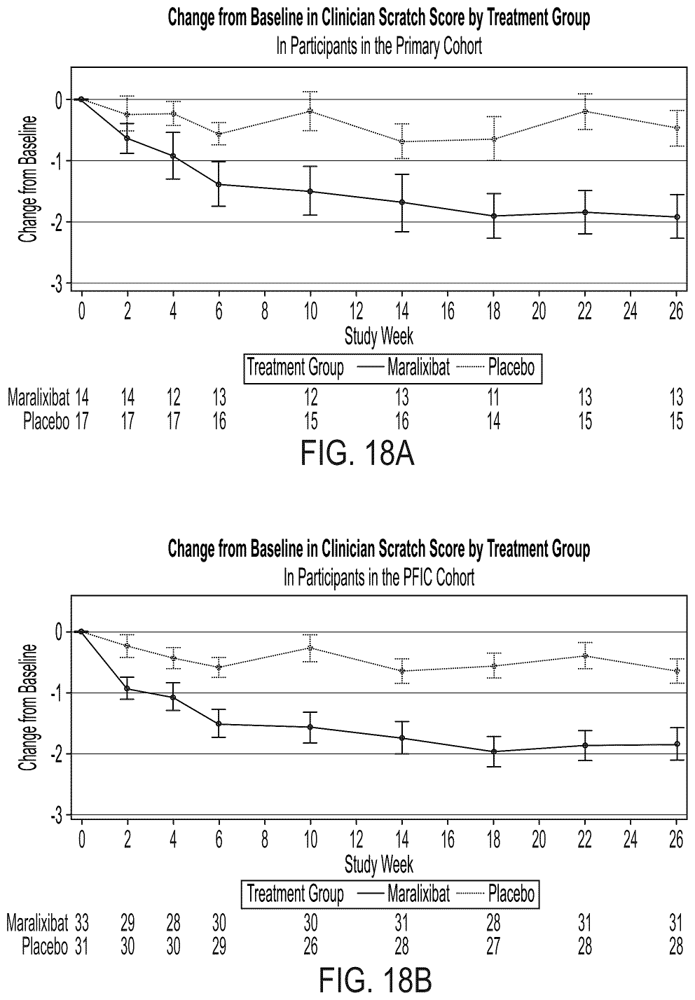

The CSS score over time for the BSEP Deficiency (aka Primary) group is shown in FIG. 18A. The CSS score over time for the PFIC group is shown in FIG. 18B. It is clear from these figures that CSS score for the maralixibat-treated participants is significantly lower at each time point from Week 2 to Week 26.

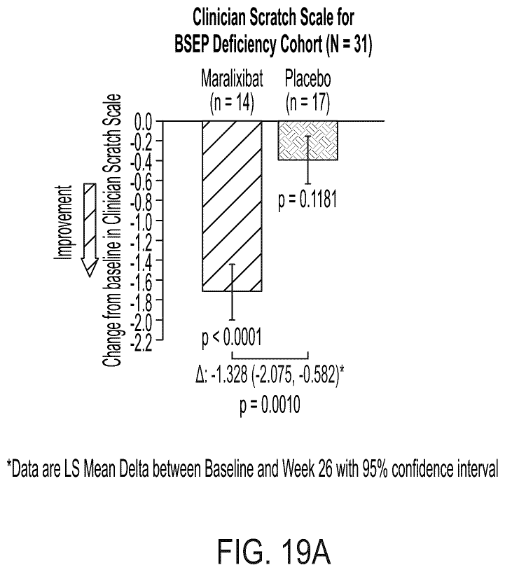

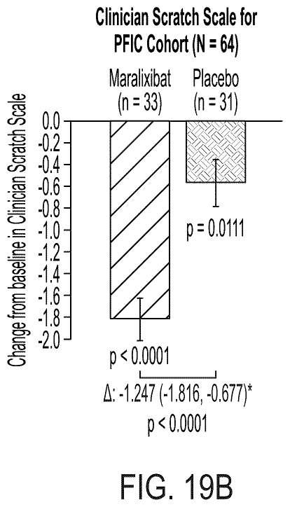

The mean change from baseline in clinician scratch scale (CSS) score is shown in FIG. 19. Estimates are from a mixed model for repeated measures (MMRM) with change from baseline as the dependent variable and fixed categorical effects of treatment group, analysis visit and treatment-by-visit interaction as well as the continuous fixed covariates of baseline score and baseline score-by-visit interaction. For the PFIC Cohort, PFIC Type is included in the model as an additional covariate. The improvement in CSS score for the BSEP deficiency cohort was about 1.7 points relative to baseline, vs. about 0.4 points for the placebo group, indicating a 1.328 point difference in improvement for the maralixibat-treated group (FIG. 19A). The improvement in CSS score for the PFIC cohort was about 1.8 points relative to baseline, vs. about 0.5 points for the placebo group, indicating a 1.247 point difference in improvement for the maralixibat-treated group (FIG. 19B).

These additional efficacy endpoint results further highlight the surprising and unexpected efficacy of maralixibat in decreasing pruritus in all PFIC patients. In addition, in a prior study of odevixibat in PFIC, efficacy measures on pruritus and serum bile acids did not increase efficacy on either endpoint. The higher dose group had lower efficacy parameters. Thus it was assumed that this was an expectation for maximal effect size in PFIC for an MAT inhibitor. The magnitude of reductions in pruritus and sBA seen in the MARCH study are larger than the odevixibat prior data.

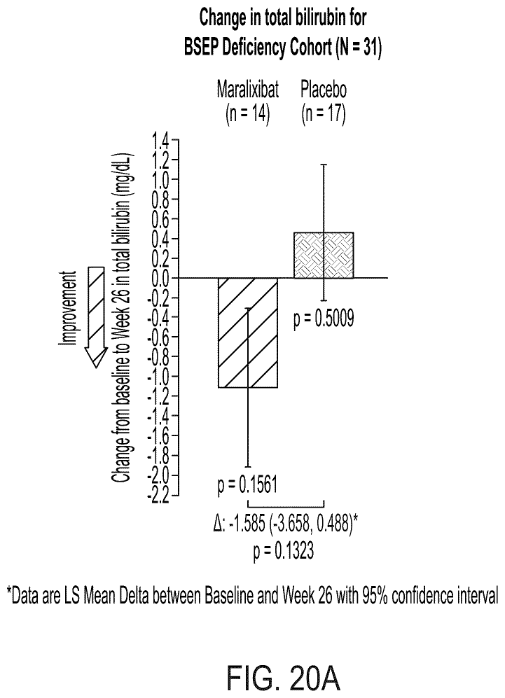

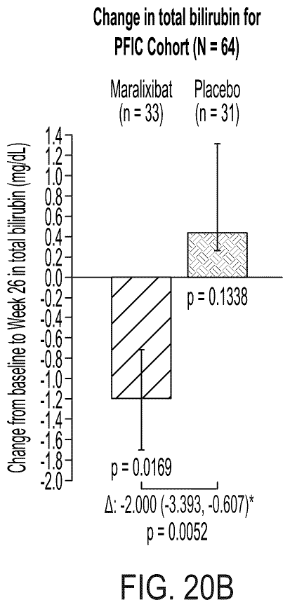

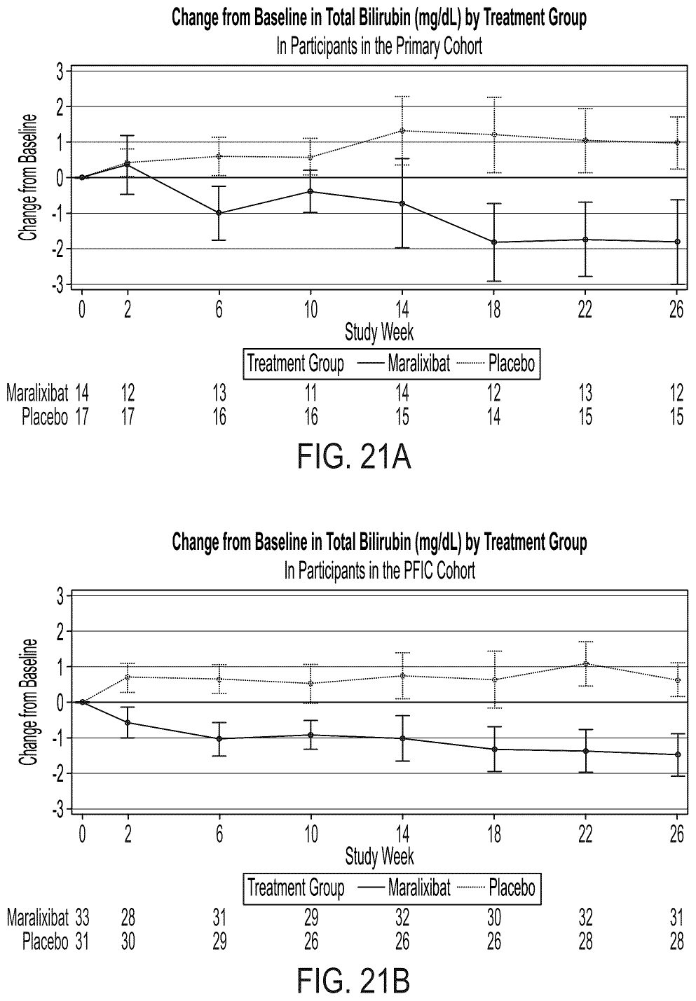

Mean change from baseline in total bilirubin is shown in FIGS. 20-21. Estimates are from a mixed model for repeated measures (MMRM) with change from baseline as the dependent variable and fixed categorical effects of treatment group, time period and treatment-by-time period interaction as well as the continuous fixed covariates of average baseline score and baseline score-by-time period interaction. [2] Average of time periods Weeks 15-18, 19-22, and 2326 obtained from the MMRM as an equally weighted average of the 3 individual visit-specific estimates. The improvement in (reduction of) total bilirubin for the BSEP deficiency cohort was about 1.2 mg/dL relative to baseline, vs. an increase of about 0.4 mg/dL for the placebo group, indicating a 1.585 mg/dL difference in improvement for the maralixibat-treated group (FIG. 20A). The improvement in (reduction of) total bilirubin for the PFIC cohort was about 1.2 mg/dL relative to baseline, vs. an increase of about 0.8 mg/dL for the placebo group, indicating a 2.000 mg/dL difference in total bilirubin improvement for the maralixibat-treated group (FIG. 20B). The change from baseline of total bilirubin over time for the B SEP Deficiency (aka Primary) group is shown in FIG. 21A. The change from baseline of total bilirubin over time for the PFIC group is shown in FIG. 21B. It is clear from these figures that total bilirubin for the maralixibat-treated participants is significantly lower (at each time point from Week 2 to Week 26 than baseline, with Week 18 and later points in time being at least 1.0 mg/dL lower than baseline.

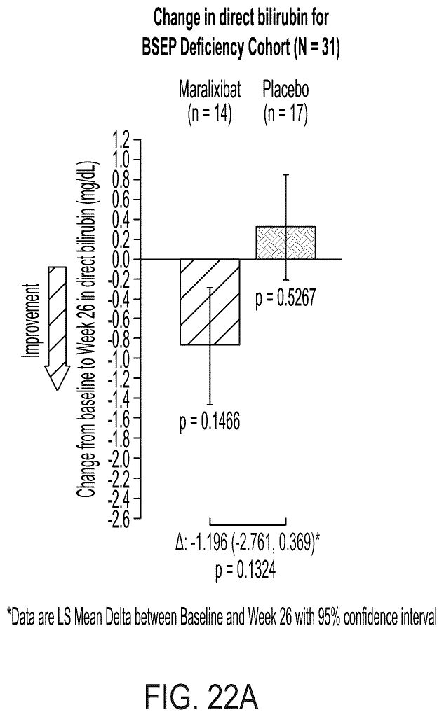

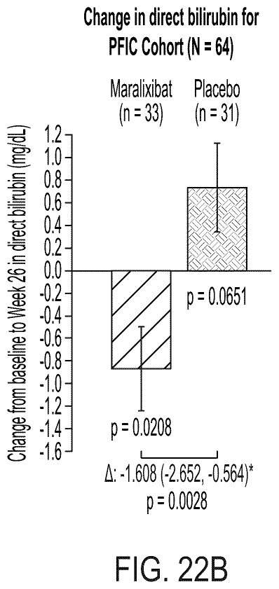

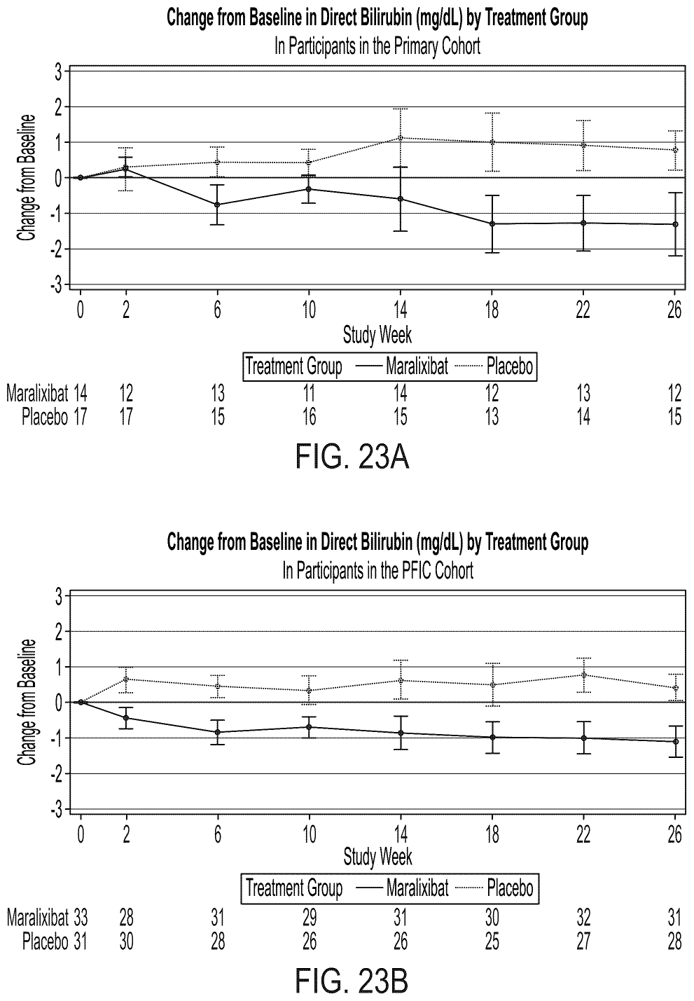

Mean change from baseline in direct bilirubin is shown in FIGS. 22-23. Estimates are from a mixed model for repeated measures (MMRM) with change from baseline as the dependent variable and fixed categorical effects of treatment group, time period and treatment-by-time period interaction as well as the continuous fixed covariates of average baseline score and baseline score-by-time period interaction. [2] Average of time periods Weeks 15-18, 19-22, and 2326 obtained from the MMRM as an equally weighted average of the 3 individual visit-specific estimates. The improvement in (reduction of) direct bilirubin for the BSEP deficiency cohort was over 0.8 mg/dL relative to baseline, vs. an increase of about 0.3 mg/dL for the placebo group, indicating a 1.196 mg/dL difference in improvement for the maralixibat-treated group (FIG. 22A). The improvement in (reduction of) direct bilirubin for the PFIC cohort was about 0.9 mg/dL relative to baseline, vs. an increase of about 0.7 mg/dL for the placebo group, indicating a 1.608 mg/dL difference in direct bilirubin improvement for the maralixibat-treated group (FIG. 22B). The change from baseline of direct bilirubin over time for the B SEP Deficiency (aka Primary) group is shown in FIG. 23A. The change from baseline of direct bilirubin over time for the PFIC group is shown in FIG. 23B. It is clear from these figures that direct bilirubin for the maralixibat-treated participants is significantly lower at each time point from Week 2 to Week 26 than baseline, with Week 18 and later points in time being at least 1.0 mg/dL lower than baseline.

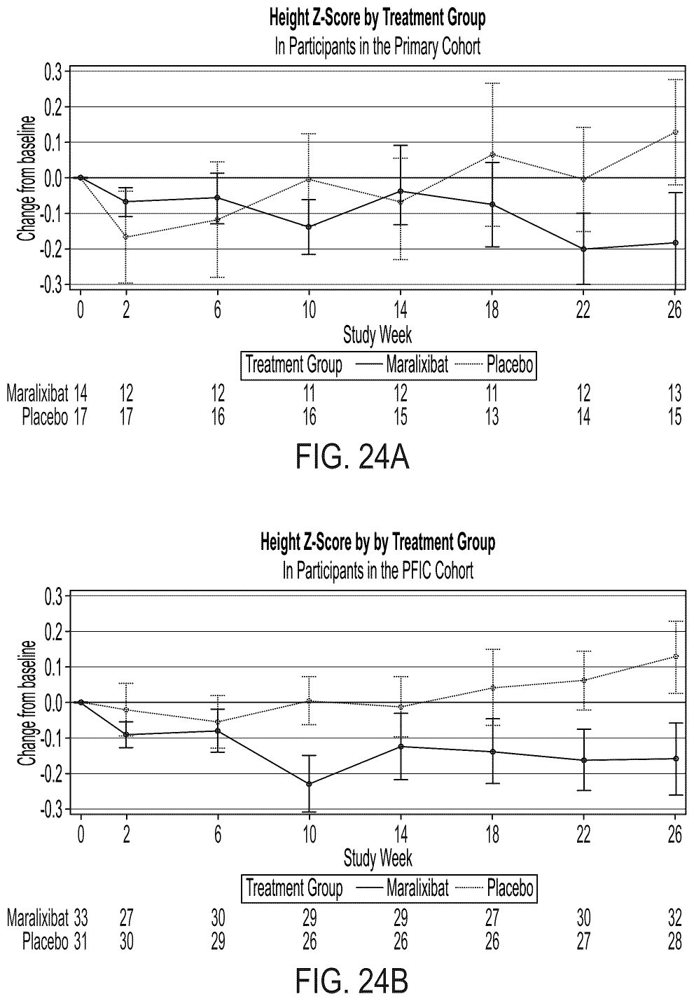

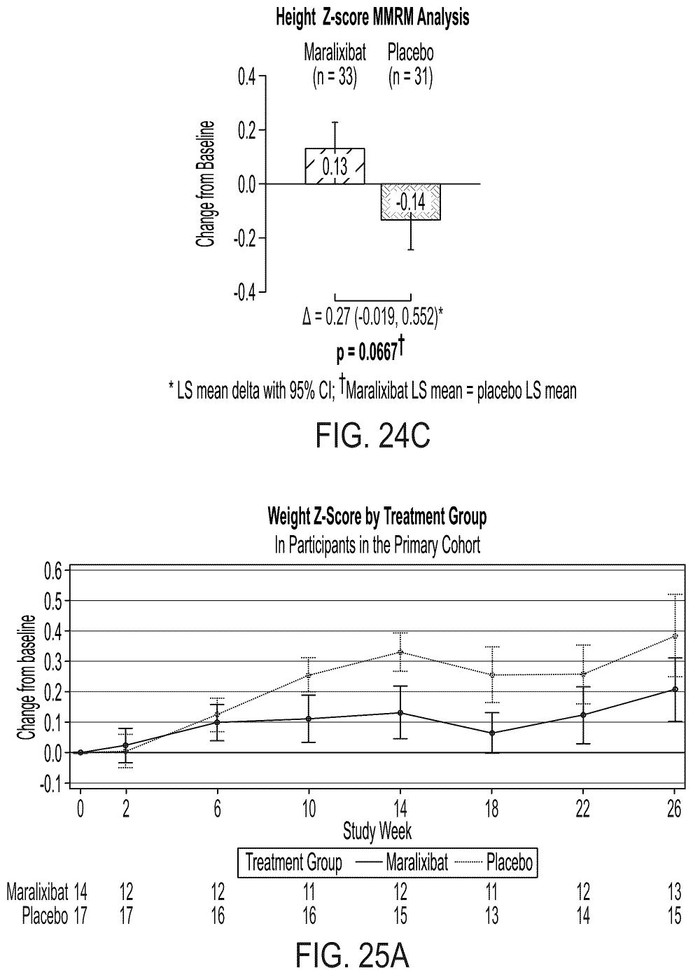

The change of participant height Z-score over time for the B SEP Deficiency (aka Primary) group is shown in FIG. 24A. The change of participant height Z-score over time for the PFIC group is shown in FIGS. 24B and 24C. These figures show an overall positive trend for increase in participant height Z-scores in the maralixibat-treated groups vs. placebo groups.

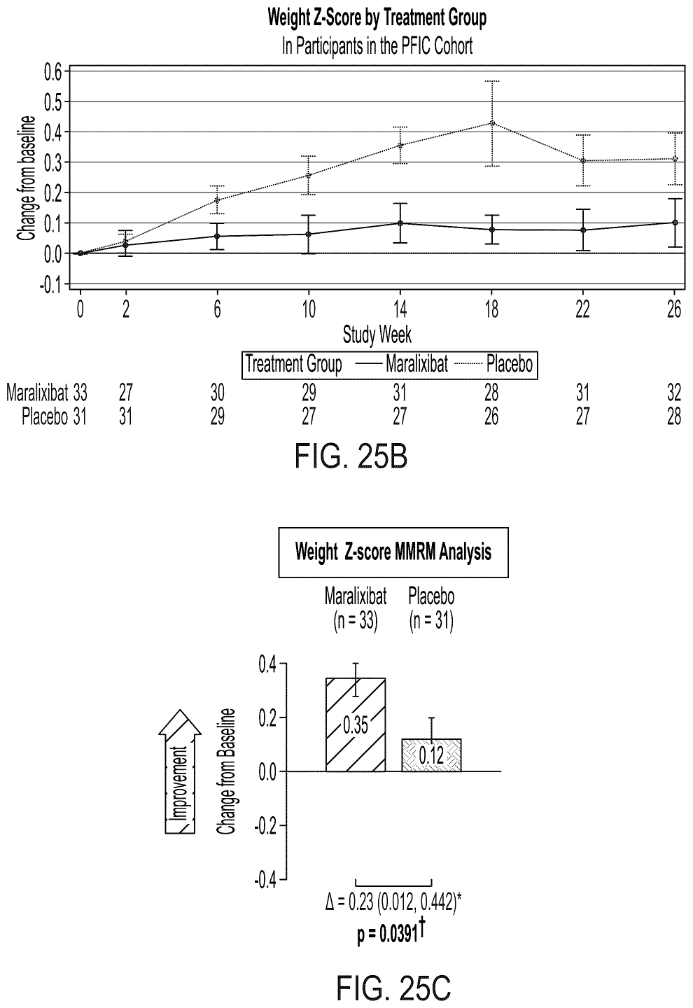

The change of participant weight Z-score over time for the B SEP Deficiency (aka Primary) group is shown in FIG. 25A. The change of participant weight Z-score over time for the PFIC group is shown in FIGS. 25B and 25C. Children in both maralixibat-treated groups and placebo groups gained weight, but it is very clear from the figures that participants in the maralixibat-treated groups gained significantly more weight over time than placebo participants.

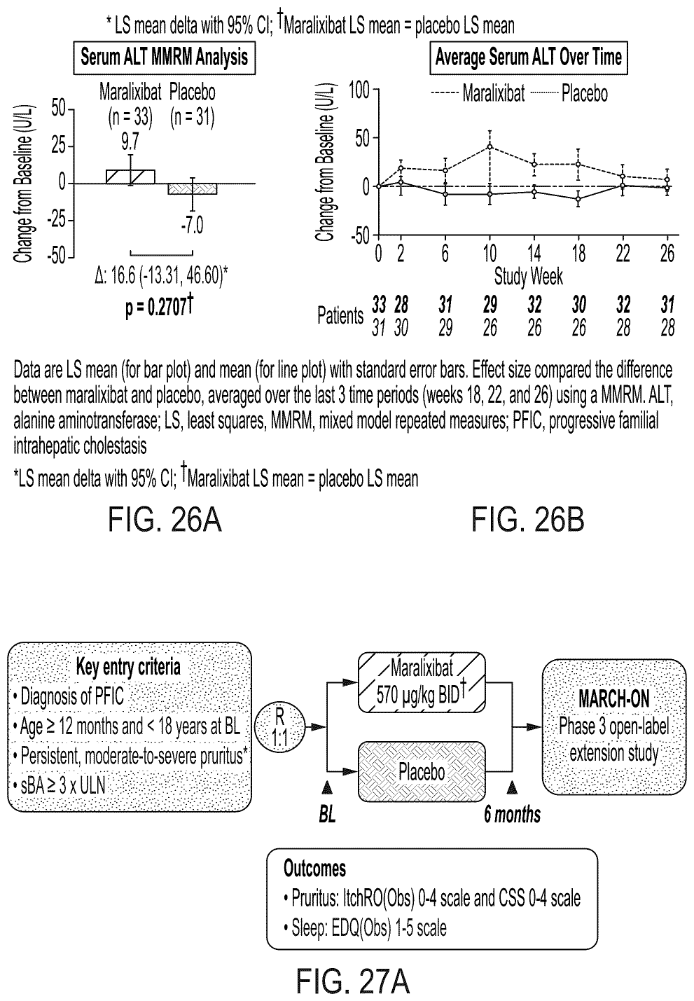

No significant changes were observed in serum ALT following MRX treatment in the All-PFIC Cohort (FIGS. 26A and 26B).

In the Indigo Phase 2 study of maralixibat, only participants with PFIC 2 had a response. Patients with PFIC 1 did not have a response. Thus the present MARCH study was designed with separate cohorts, because the other (non-PFIC 2) subtypes were not expected to have a response.

However, surprisingly, significant improvements in sBA, pruritus, total and direct bilirubin were all observed in the present study across all PFIC types.

Furthermore, in the non PFIC 2 patients, the magnitude of the effect on sBA reduction and pruritus reduction was the same or stronger than in PFIC 2 patients, which is unexpectedly contrary to prior study results.

Another surprising finding is that the group in the full population incudes not only the other PFIC subtypes but also PFIC patient post surgery. One would not expect these patients to respond at all given that surgery has the same mechanism to interrupt the enterohepatic circulation. However, these patients unexpectedly showed a significant response to maralixibat treatment.

### Pharmacokinetic Analysis

Systemic concentrations of maralixibat in plasma were determined at pre-dose and at approximately 2.5 hours after the morning dose at Week 10 and Week 26 (EOT/ET). Summary statistics (number of observations, mean, standard deviation, coefficient of variation, median, minimum, maximum, and geometric mean) were determined for maralixibat concentrations at each nominal time point and sampling time.

### Example 5. Adverse and Serious Adverse Events Assessment

An Adverse Event (AE) or Treatment-Emergent Adverse Event (TEAE) is any untoward medical occurrence in a clinical investigation subject administered a pharmaceutical product and that does not necessarily have a causal relationship with this treatment. A TEAE can therefore be any unfavorable and unintended sign (including an abnormal laboratory finding), symptom, or disease temporally associated with the use of a medicinal (investigational) product, whether or not related to the medicinal (investigational) product (ICH Guidance E2A 1995). All TEAEs are collected from the time the informed consent is signed until the defined follow-up period. This includes events occurring during the screening phase of the study, regardless of whether or not study medication is administered.

Table 6, below, summarizes TEAEs across all PFIC cohorts for the MARCH Phase 3 study. The most common TEAEs were GI disorders [n (%)]35 (74.5%) for Maralixibat and 17 (37.0%) for Placebo. In total, 90 patients experienced ≥1 TEAE, with 84 being either mild or moderate in severity (93.3%) and transient in nature. The maximum grade experienced was mild or moderate in 84 out of the 90 participants with AE.

TABLE 6
Summary of TEAEs Across All PFIC Cohorts

| | Maralixibat | | Placebo | |
|---|---|---|---|---|
| Adverse event | (n = 47) | | (n = 46) | |
| Any TEAE, n (%) | 47 | (100%) | 43 | (93.5%) |
| Grade 3/4 TEAE, n (%) | 3 | (6.4%) | 3 | (6.5%) |
| Serious TEAE, n (%) | 5 | (10.6%) | 3 | (6.5%) |
| TEAE leading to discontinuation, n (%) | 1 | (2.1%) | 0 | |
| TEAE leading to death, n (%) | 0 | | 0 | |
| TEAE potentially related to study | 18 | (38.3%) | 2 | (4.3%) |
| drug, n (%) | | | | |
| GI events potentially related to study | 14 | (29.8%) | 2 | (4.3%) |
| drug, n (%) | | | | |
| Diarrhoea | 27 | (57.4%) | 9 | (19.6%) |
| Diarrhoea related to study drug n (%) | 13 | (27.7%) | 1 | (2.2%) |

A serious adverse event (SAE) is any untoward medical occurrence (whether considered to be related to study medication or not) that at any dose: 1) Results in death; 2) Is life-threatening; 3) Requires inpatient hospitalization or prolongation of existing hospitalization; 4) Results in persistent or significant disability/incapacity; or 5) Is an important medical event.

All SAEs (regardless of relationship to study) are collected from the time the subject signs the informed consent until the defined follow-up period and must be reported within 24 hours of the first awareness of the event.

Of 47 maralixibat-treated participants, 5 experienced an SAE (10.6%). Of 46 placebo participants, 3 experienced an SAE (6.5%).

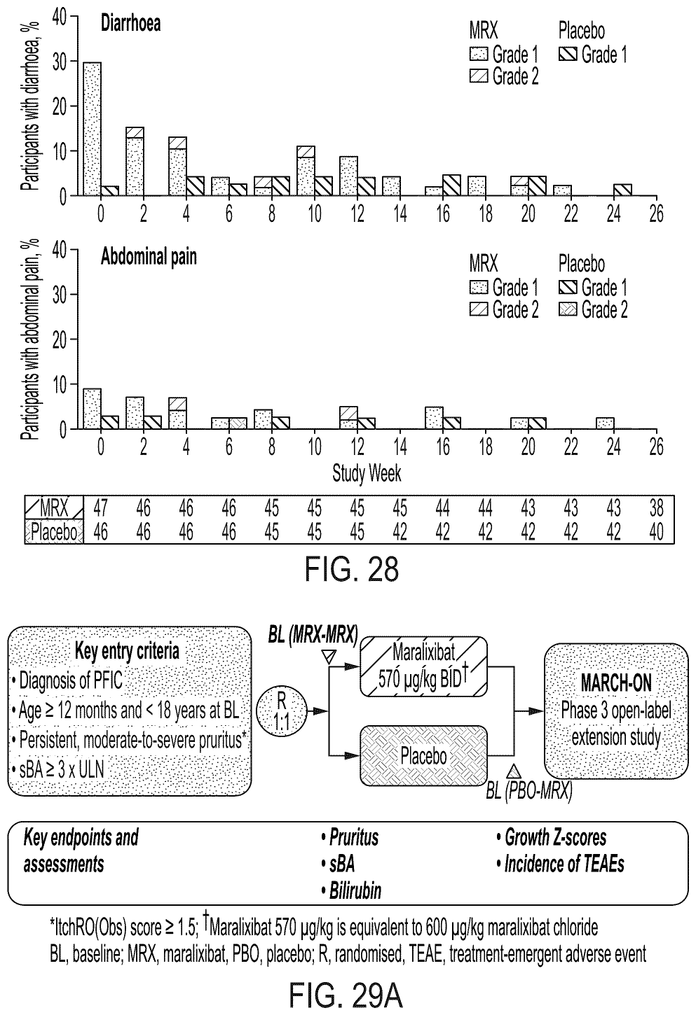

TEAEs included diarrhea (57.4% vs 19.6%) and abdominal pain (25.5% vs 13%) for MRX vs PBO, respectively (Table 7). Diarrhea was mostly grade 1, transient, with a median duration of 5.5 days; there were no severe or serious events. One patient with mild diarrhea discontinued therapy (Table 6 and FIG. 28). Abdominal pain was also mostly mild and transient and, in nearly all instances, was concurrent with diarrhea. There were no clinically meaningful changes observed in either group from baseline in transaminase levels.

TABLE 7
TEAEs of Clinical Interest Occurring in ≥5% of
Participants in Either Arm, by FMQ and Preferred Term

| | | |
|---|---|---|
| TEAE, n (%) | MRX (n = 47) | Placebo (n = 46) |

| | | | | |
|---|---|---|---|---|
| Gastrointestinal | | | | |
| Diarrhoea | 27 | (57.4) | 9 | (19.6) |
| Abdominal pain | 12 | (25.5) | 6 | (13) |
| Constipation | 4 | (8.5) | 2 | (4.3) |
| Gastroenteritis | 3 | (6.4) | 2 | (4.3) |
| Haematochezia | 3 | (6.4) | 11 | (2.2) |
| Vomiting | 3 | (6.4) | 5 | (10.9) |
| Liver function tests | | | | |
| Transaminase AEs | 8 | (17) | 3 | (6.5) |
| Increased ALT | 6 | (12.8) | 3 | (6.5) |
| Hyperbilirubinemia | 7 | (14.9) | 9 | (19.6) |
| FSV deficiency | 13 | (27.7) | 16 | (34.8) |

| | | | |
|---|---|---|---|
| Fractures | 3 | (6.4) | 0 |

Transaminase adverse events (AEs) were observed in 17% and 6.5% of MRX and PBO patients, respectively (Table 7). Among the 8 participants receiving MRX who had transaminase elevations, 6 had resolution of the elevation without drug interruption; 2 patients had ongoing stable elevation even after drug interruption (n=1) or dose reduction (n=1), and both ultimately resumed prior maximum dose. No patients discontinued MRX due to transaminase elevation.

There was no evidence to suggest that MRX treatment contributes to Fat Soluble Vitamin (FSV) deficiency. FSV deficiency, which was reported as an AE, was also less common in MRX vs PBO (27.7% vs 34.8%). No clinically meaningful changes from baseline in setrum levels of vitamins A, D, and E were observed in the study population under regular FSV supplementation due to chronic cholestasis. Bilirubin increase was less common in MRX vs PBO (14.9% vs 19.6%). International normalized ratio (INR) decreased (improved) at every timepoint assessed with a mean change from Baseline of −0.3 for MRX vs −0.03 for placebo at week 26.

Fractures were seen in 6.4% of MRX and in 0% of PBO; none were considered treatment related as all had clear alternative causes for fracture, including pre-existing vitamin D deficiency which was stable or improved on MRX. Serious AEs were reported in 10.6% of MRX and 6.5% of PBO patients; none were deemed related (except 1 event of mild bilirubin increase in MRX); all resolved without any dose modifications.

MRX was well-tolerated, with GI effects being the most frequent event but generally mild and self-limiting. FSV deficiency and bilirubin increase were more frequently seen in the PBO group. Overall, no change in liver enzymes were observed over the duration of the study, and individual elevations were mild and transient; there was no discontinuations.

### Example 6. Safety Monitoring of Selected Parameters

### Liver Parameters

Table 8 provides the criteria for close monitoring of liver parameters.

For subjects with a confirmed increase in ALT or TSB level, meeting the close monitoring criteria, the following investigations were considered, as clinically indicated: 1) Close and frequent monitoring of liver enzyme and serum bilirubin tests as clinically indicated; 2) Symptoms as well as prior, current, and intercurrent diseases/illnesses; 3) Use of or recent change in use of concomitant treatment (including non-prescription medications, herbal and dietary supplement preparations), alcohol use, recreational drug use, and special diets; 4) History for exposure to environmental chemical agents and travel; 5) Serology for viral hepatitis (HAV IgM, HBsAg, HCV antibody, HCVRNA, CMV IgM, and EBV antibody panel); 6) Serology for autoimmune hepatitis (e.g., antinuclear antibody [ANA]); 7) AST, creatine phosphokinase (CPK), and lactate dehydrogenase (LDH); 8) CBC with differential blood count (eosinophils); 9) Reticulocyte count, PT/INR.

TABLE 8
Close Monitoring Criteria for Treatment-
Emergent Elevated ALT and TSB

| Parameter | Baseline Value | Close Monitoring Criteria |
|---|---|---|
| ALT | ≤30 U/L | ≥100 U/L |
| | >30 to ≤150 U/L | >3 × BL or ≥250 U/L |
| | | whichever comes first |
| | >150 to ≤450 U/L | ≥(BL + 100 U/L) |
| TSB | Any | ≥(BL + 3 mg/dL) |

ALT = alanine aminotransferase; BL = baseline; TSB = total serum bilirubin.

### Example 7. Additional Results for FIC1, MDR3, TJP2, and MYO5B Cohorts

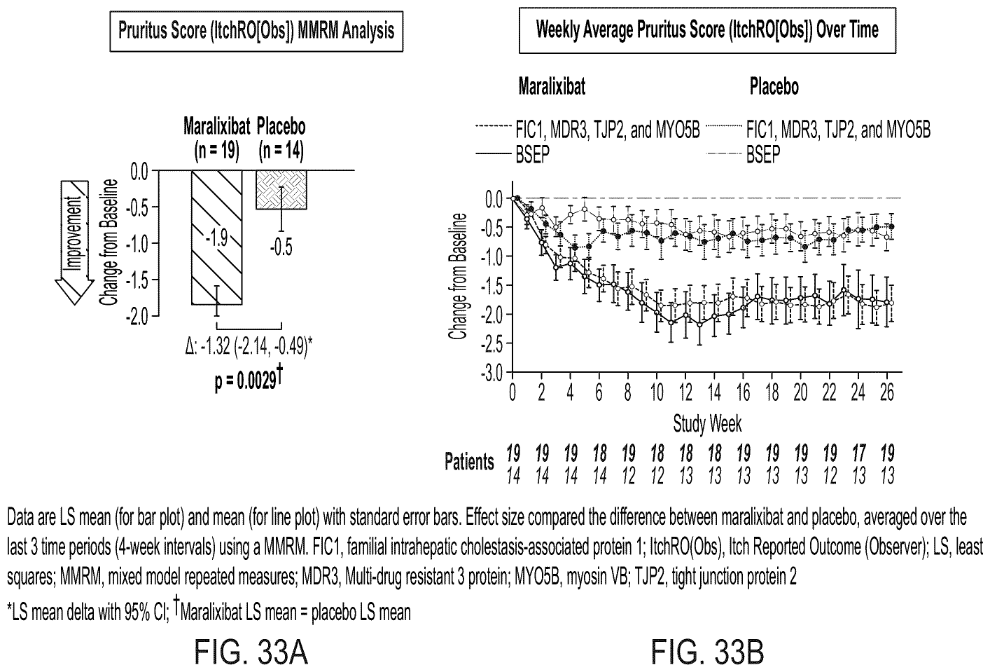

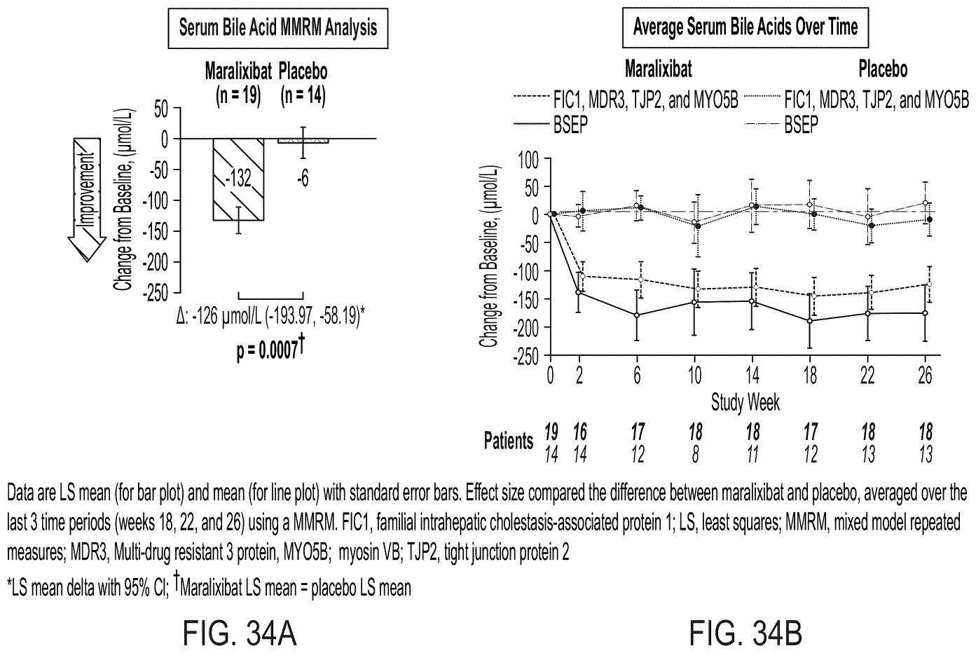

Treatment with MRX resulted in statistically significant and clinically meaningful improvements in pruritus in in familial intrahepatic cholestasis-associated protein 1 (FIC1), multidrug resistant 3 protein (MDR3), tight junction protein 2 (TJP2), myosin VB (MYO5B) cohorts (FIGS. 33A and 33B). Significant improvements in serum bile acid levels were observed in FIC1, MDR3, TJP2, and MYO5B cohorts (FIGS. 34A and 34B).

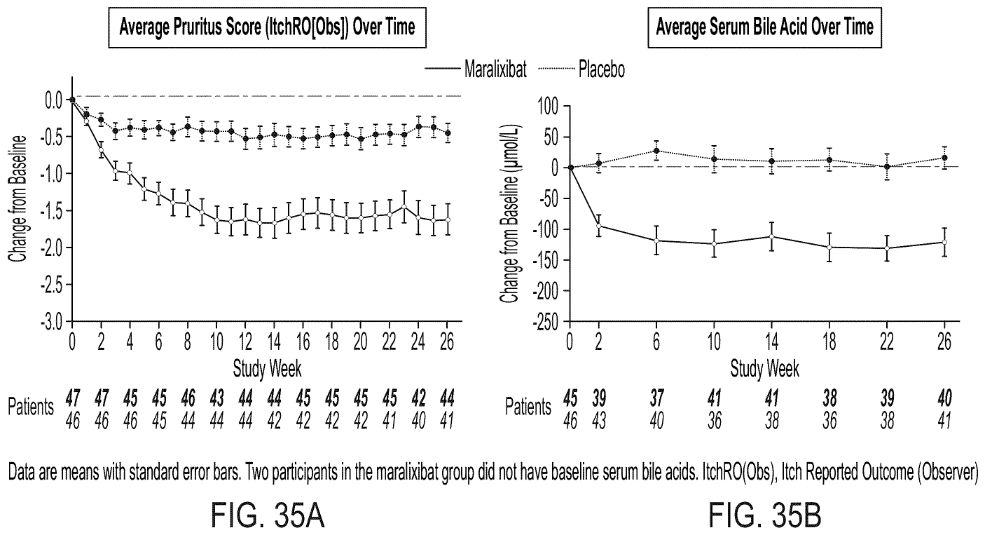

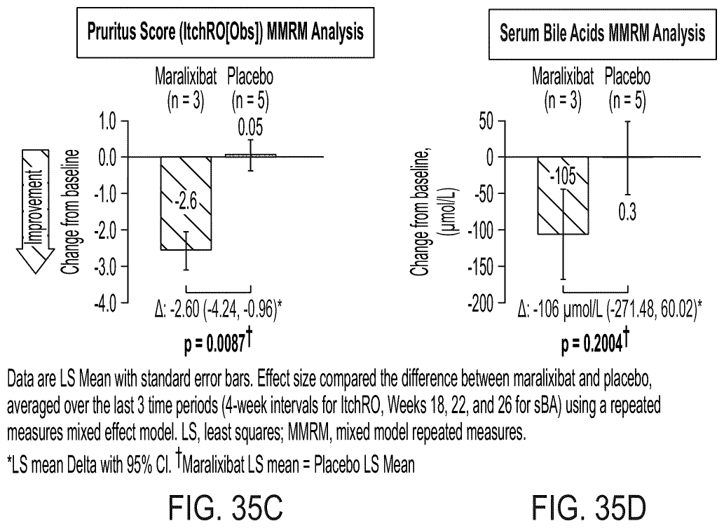

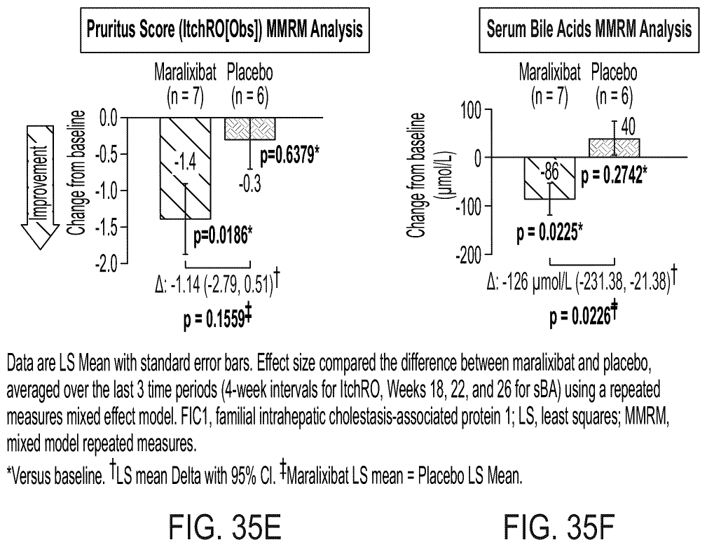

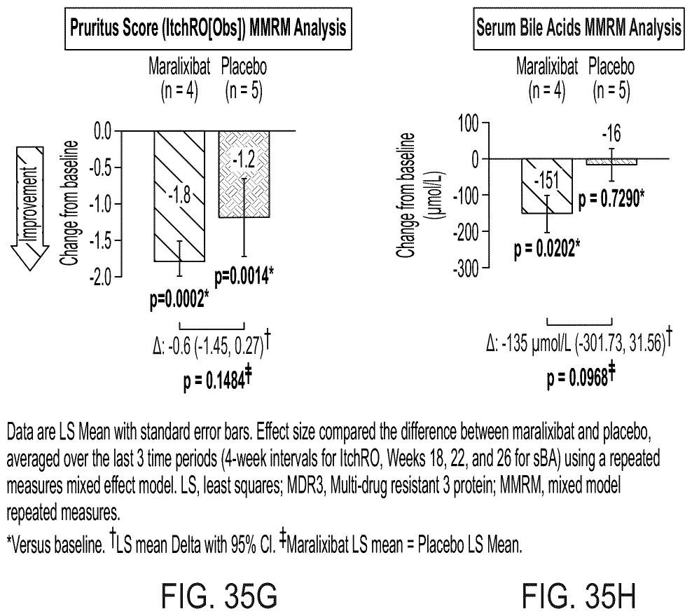

Treatment with MRX resulted in statistically significant and clinically meaningful improvements in pruritus severity and serum bile acid levels across the full-study population (FIGS. 35A and 35B). In patients with no-variant found, MRX demonstrated improvements in pruritus and serum bile acid levels (FIGS. 35C and 35D). Change in weekly ltchRO(Obs) score and serum bile acids (sBA) were also observed in FIC1 (FIGS. 35E and 35F) and MDR3 (FIGS. 35G and 35H) cohorts.

### Example 8. Maralixibat Leads to Significant Reductions in Pruritus and Improvements in Sleep for Children with Progressive Familial Intrahepatic Cholestasis

Methods: Participants were randomized to receive MRX 570 μg/kg BID or placebo (PBO) (FIG. 27A). The primary analysis evaluated pruritus as assessed by the caregiver using ItchRO(Obs) 0-4 scale; as well as by physician using the Clinician Scratch Scale (CSS). Sleep was assessed using the Exploratory Diary Questionnaire (Observer) (EDQ[Obs]). Responses for pruritus and sleep were determined as the average in weekly score using a mixed-effects model with recurrent measurements for Change from Baseline (CFB) through Week 26.

Itch-Reported-Outcome (Observer) (ItchRO(Obs)) was on a 0-4 scale, where 0=no itch, 1=moild, 2=moderate, 3=severe, and 4=very severe. A ≥1-point reduction in ItchRO(Obs) is considered clinically meaningful.

Clinician Scratch Scale (CSS) was scored on a 0-4 scale, where 0=none and 4=cutaneous mutilations, haemorrage, and scarring (worst scratching).

Exploratory Diary Questionnaire (Observer) (EDQ[Obs]) was on a 1-5 scale (1=never/no itch to 5=almoust always/very severe) that includes a question on sleep disturbances related to pruritus.

Results: 64 patients (nt-BSEP [n=31], FIC1 [n=13], MDR3 [n=9], TJP2 [n=7], and MYO5B [n=4]) were randomized to MRX (n=33) or PBO (n=31). Baseline characteristics were well-balanced between treatment arms (Table 9). Baseline pruritus score (ItchRO[Obs]) was 2.9 vs 2.7 for MRX and PBO. From Baseline to Week 26, the proportion (SE) of pruritus assessments ≤1 was greater in MRX (0.62 [0.06]) vs PBO (0.27 [0.06]) groups and the difference was significant (delta [95% CI]: 0.35 [0.19, 0.50]) (FIG. 27B). From Weeks 15-26, the median proportion of reported days with an ItchRO(Obs) score of 0-1 was 95% for MRX and 9% for PBO (p=0.0005). Pruritus response from Weeks 15-26 using ItchRO(Obs) was greater in the MRX group when measured in the morning (delta [95% CI]: −1.20 [−1.73, −0.67]), evening (delta: −1.16 [−1.69, −0.63]), or max daily (delta: −1.20 [−1.74, −0.66]) (FIG. 27C). Pruritus response measured with the CSS was greater in MRX (delta: −1.8 [−2.2, −1.53]) vs PBO (delta: −0.7 [−1.1, −0.3]) groups and the difference was significant (delta: −1.1 [−1.7, −0.6]; p<0.0002). The CFB in sleep was greater in MRX (delta: −1.74 [−2.15, −1.33]) vs PBO (delta: −0.55 [−0.99, −0.11]) groups and the difference was significant (delta:−1.19 [−1.78, −0.60]; p=0.0002) (FIG. 27D). Throughout the study, there was a strong correlation between absolute values in pruritus and sleep scores (Spearman's r=0.93; p<0.0001) and also between change for Baseline for pruritus and sleep (Spearman's r=0.93; p<0.0001) (FIG. 27E).

TABLE 9
Baseline Characteristics Between Treatment Arms

| | Maralixibat | Placebo |
|---|---|---|
| Variable | (n = 33) | (n = 31) |
| Age, years | 4.9 | 4.4 |
| Male, % | 52 | 42 |
| Pruritus, ItchRO(Obs) score | 2.85 | 2.73 |
| Baseline CSS score | 2.8 | 2.6 |
| Baseline EDQ(Obs) sleep disturbance | 3.70 | 3.66 |
| Total sBA, pmol/L | 254 | 272 |
| UDCA usage, % | 82 | 97 |
| Rifampicin usage, % | 55 | 48 |
| Alanine aminotransferase, U/L | 88 | 127 |
| Total bilirubin, mg/dL | 4.12 | 4.04 |
| Direct bilirubin, mg/dL | 2.98 | 2.93 |
| Height Z-score | −2.08 | −2.06 |
| Weight Z-score | −1.75 | −1.28 |

All data are mean unless otherwise indicated. Percentages are 100×n/N. UDCA is urodeoxycholic acid.

Conclusions: MRX was associated with complete or near-complete resolution of pruritus in the majority of patients with PFIC, and the effect was independent of how it was measured, or who made the assessments. Changes with pruritus were strongly correlated with changes in sleep, suggesting that use of MRX may yield meaningful improvements in this domain of quality of life.

### Example 9. Long-Term Maintenance of Response and Improved Liver Health with Maralixibat in Patients with Progressive Familial Intrahepatic Cholestasis (PFIC)

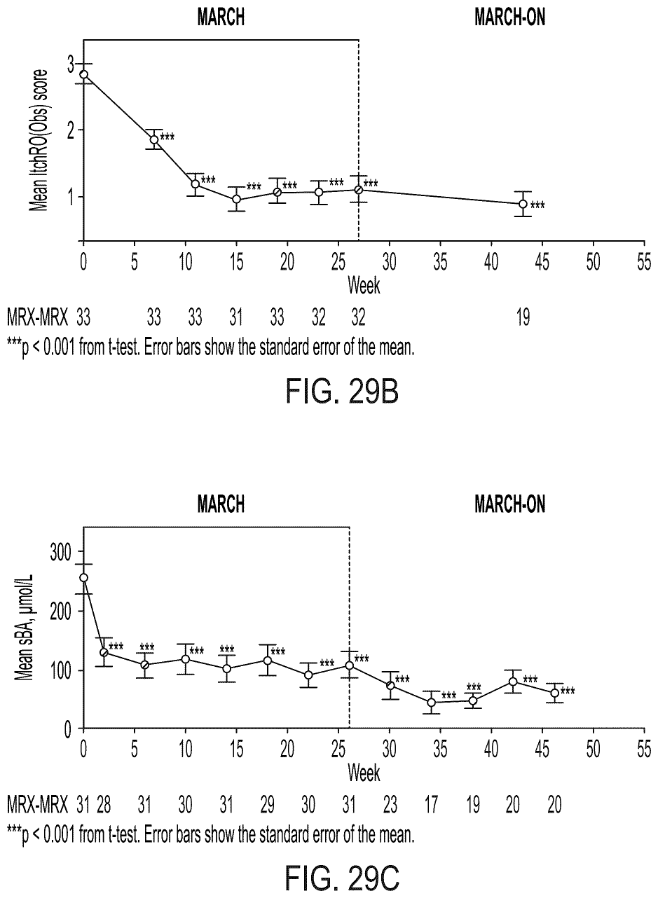

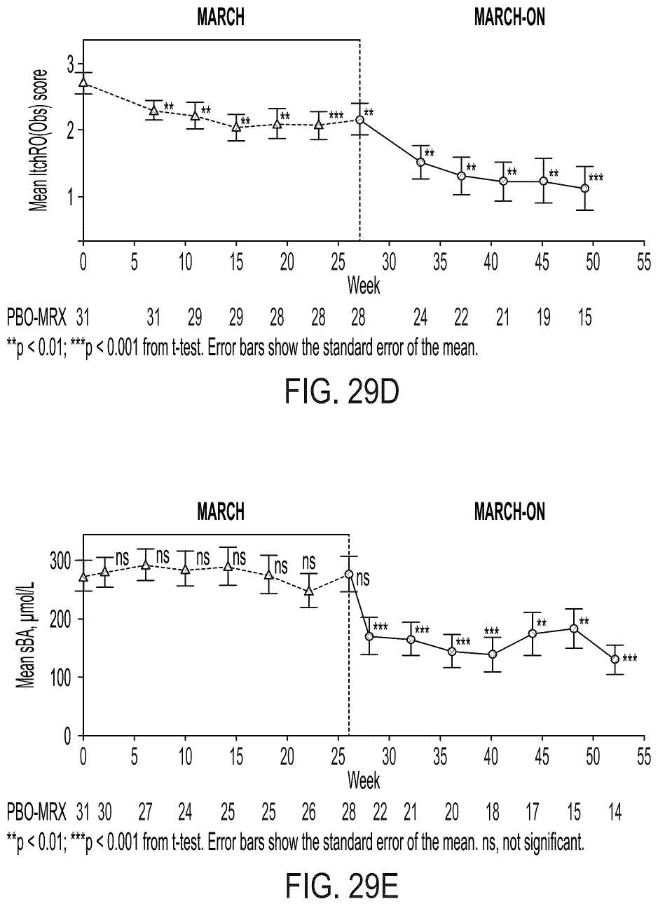

Eighty-five patients from MARCH trial enrolled in MARCH-ON (Table 10, FIG. 29A). Forty-seven patients had received Maralixibat (MRX-MRX) and 38 had received placebo (PBO-MRX) during MARCH trial.

TABLE 10
Baseline Characteristics Between Treatment Arms

| | MRX-MRX | PBO-MRX |
|---|---|---|
| Variable | (n = 47) | (n = 38) |
| Age, years | 4.8 | 5.1 |
| Male, % | 43 | 42 |
| Pruritus, ItchRO(Obs) score | 2.8 | 2.5 |
| Total sBA, pmol/L | 263 | 253 |
| UDCA usage, % | 83 | 82 |
| Rifampicin usage, % | 55 | 58 |
| Alanine aminotransferase, U/L | 108 | 102 |
| Total bilirubin, pmol/L | 70 | 77 |
| Direct bilirubin, pmol/L | 51 | 57 |
| Height Z-score | −1.9 | −2.0 |
| Weight Z-score | −1.5 | −1.2 |

All data are mean unless otherwise indicated. Percentages are 100×n/N. UDCA is urodeoxycholic acid.

PFIC subtypes of patients included in the study were: non-truncated bile salt export pump (nt-B SEP, n=27), familial intrahepatic cholestasis-associated protein type 1 (F1C1, n=13); multidrug-resistance 3 protein (MDR3, n=9); tight junction protein 2 (TJP2, n=6); myosin VB (MYO5B, n=2); heterozygosis (n=2); truncated B SEP (t-B SEP, n=9); variant not found (n=8), fluctuating sBA (n=2); and surgery (n=7). Efficacy analyses included n=33 in the MRX-MRX group and n=24 in the PRO-MRX group. Subtypes nt-BSEP, F1C1, MDR3, TJP2, and MYO5B were included in the efficacy analyses. Baseline was defined as the start of maralixibat (MRX) treatment for each group.

Methods: Long-term maintenance of response was assessed for patients who were originally randomized to receive MRX in MARCH and continued with treatment in MARCH-ON (MRX-MRX group; n=33). MRX response was assessed for patients who received PBO in the MARCH study and switched to open-label MRX in MARCH-ON (PBO-MRX group; n=24). Assessments included: pruritus measured by 0-4 scale of ItchRO[Obs], sBA, bilirubin, and growth z-scores, as well as incidence of treatment-emergent adverse events(TEAEs). Baseline (BL) was defined as the start of MRX for each group.

Results: For the MRX-MRX group, the median (min, max) time on MRX was 394 days (108, 836). In total, 20 of 33 patients reached Week 52 at time of analysis. Significant improvements observed in the first 26 weeks of the MARCH study were sustained from Baseline (BL) through Week 52 in MARCH-ON for pruritus severity (−2.17, p<0.0001), sBA (−200 p=0.0004), bilirubin (−2.64 mg/dL, p=0.0084), height z-score (+0.54, p<0.0001), and weight z-score (+0.44, p=0.0010) (FIG. 29B-C, Table 11). In the PBO-MRX group, the median time on MRX was 256 days (29,569). In total, 15 of 24 patients reached Week 26 at time of analysis. Newly gained statistically significant reductions in pruritus and sBA levels were observed in the key efficacy end points from BL through Week 26 for pruritus (−1.05, p=0.0017) and sBA (−141 μmol/L, p=0.0003) (FIG. 29D-E, Table 11), in line with observations from the initial MARCH MRX group. Overall, there were no new safety signals identified (Table 12). The most frequent TEAEs were gastrointestinal (GI)-related with early onset of diarrhea (51%) in line with the mechanism of MAT inhibition, mostly mild and transient. In the MRX-MRX sub-group, fewer patients experienced diarrhea in MARCH-ON supporting these effects are early and transient in nature.

TABLE 11
Significant Improvements in Key Endpoints were Observed
from Baseline to Week 52 in the MRX-MRX group and
Basemine to Week 26 in the PBO-MRX group

| | MRXMRX | | PBO-MRX | |
|---|---|---|---|---|
| Mean change from Baseline | (n = 20)^{a} | | (n = 15)^{b} | |
| Pruritus. ItchRO(Obs) score | −2.17 | (p < 0.0001) | −1.05 | (p = 0.0017) |
| sBA, μmol/L | −200 | (p = 0.0004) | −141 | (p = 0.0003) |
| Total bilirubin, μmol/dL | −45 | (p = 0.0084) | −27 | (p = 0.1878) |
| Height Z-score | +0.54 | (p < 0.0001) | +0.46 | (p = 0.0152} |
| Weight Z-score | +0.44 | (p = 0.00101) | +0.09 | (p = 0.5134) |

^{a}Analysis includes n = 20 patients in the MRX-MRX group with follow-up to Week 52.
^{b}Analysis includes n = 15 patients in the PBO-MRX group with follow-up to Week 26.

TABLE 12
No New Safety Signals were Identifies
During Treatment with Maralixibat

| | | |
|---|---|---|
| | MRX-MRX | PBO-MRX |
| TEAEs, n(%) | (n = 47) | (n = 38) |

| Any TEAE | 47 | (1M) | 35 | (92.1) |
|---|---|---|---|---|
| Severe TEAE | 5 | (10.6) | 2 | (5.3) |
| Serious TEAE | 8 | (17.0) | 6 | (15.0) |
| TEAE leading to discontinuation | 3 | (6.4) | 0 | |
| TEAE leading to death | 1 | (2.1) | 0 | |
| Most common TEAE: diarrhoea | 30 | (63.8) | 13 | (34.2) |

Percentages are 100×n/N. TEAE is treatment-emergent adverse effect.

Conclusions: Significant and sustained responses in pruritus, sBA, bilirubin as well as growth are observed with 52 weeks of MRX treatment across the broadest range of genetic PFIC types studied to date. The PBO-MRX group demonstrated significant improvements in pruritus severity and sBA levels similar to those observed in the original MARCH maralixibat group. These data suggest overall improved liver health with MRX treatment which can be maintained over time.

### Example 10. Impact of Maralixibat (MRX) on Cholestatic Pruritus in Adults Aged 16 Years and Older with Alagille Syndrome (ALGS)

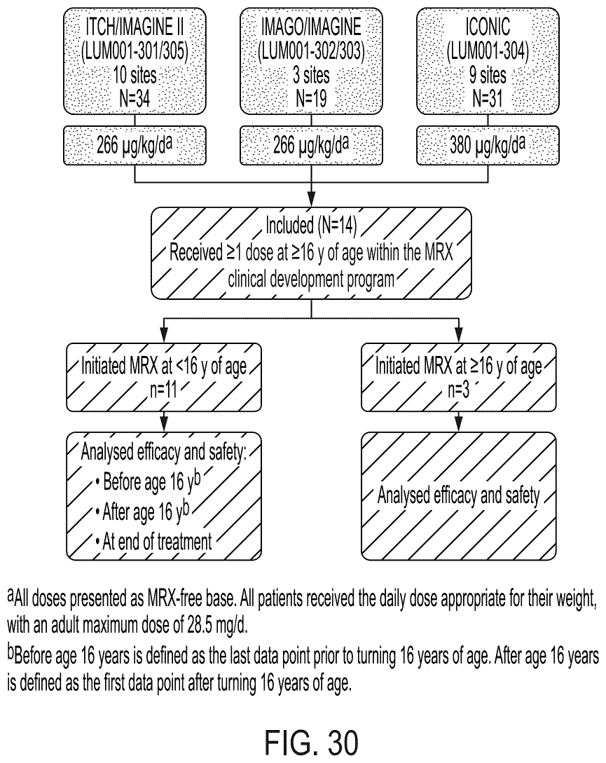

The efficacy and safety of MRX in participants with ALGS aged ≥16 years transitioning to adult care and participant aged ≥16 years who initiate MRX treatment were studied (FIG. 30). The median (min, max) duration of therapy for participants who initiated MRX at <16 years of age was 4.1 years (1.5, 5.9), with the oldest patient taking MRX at 21 years of age (Table 13). Three participants began MRX at ≥16 years of age and were followed for a median of 3.8 years.

TABLE 13
Key Demographics and Baseline Characteristics (N = 14)

| | | |
|---|---|---|
| | Participants <16 y | Participants >16 y |
| Variable Mean (SE) | at MRX initiation | at MRX initiation |
| Unless specified | (n = 11) | (n = 3) |

| | | | | |
|---|---|---|---|---|
| Age, y | 13.2 | (0.4) | 16.3 | (0.3) |

| | | |
|---|---|---|
| Sex, male, % | 45.5 | 66.7 |

| | | | | |
|---|---|---|---|---|
| Pruritus, ItchRO(Obs) | 2.5 | (0.2) | 3.1 | (0.4) |
| Total sBA, μmol/L | 130 | (39) | 82 | (57) |
| Total bilirubin, mg/dL | 36.4 | (12.6) | 27.9 | (14.3) |
| Direct bilirubin, mg/dL | 28.1 | (11.5) | 17.7 | (10.0) |
| Height z score | −1.8 | (0.5) | −1.0 | (0.3) |
| Weight z score | −1.8 | (0.3) | −1.1 | (0.4) |
| ALT, U/L | 174 | (52) | 106 | (12) |

ALT is alanine aminotransferase. Adverse events (AEs) decreased as patients got older (Table 14).

TABLE 14
Summary of treatment-emerged adverse effects
(TEAEs) in full study cohort (N = 14).

| | Participants aged <16 y | | | | Participants | |
|---|---|---|---|---|---|---|
| | at MRX initiation | | | | aged >16 y at | |
| | (n = 11)^{a} | | | | MRX initiation | |
| TEAE, n (%) | Before 16 y | | After 16 y | | (n = 3) | |
| Any TEAEs | 11 | (100) | 7 | (63.6) | 3 | (100) |
| TEAEs > grade 3 | 1 | (9.1) | 1 | (9.1) | 1 | (33.3) |
| Treatment-related AEs | 9 | (81.8) | 2 | (18.2) | 2 | (66.7) |
| Treatment-related | 0 | | 0 | | 1 | (33.3) |
| AEs ≥ grade 3^{b} | | | | | | |
| SAEs | 2 | (18.2) | 1 | (9.1) | 0 | |
| Treatment-related SAEs | 0 | | 0 | | 0 | |
| AEs leading to study drug | 0 | | 0 | | 0 | |
| discontinuation | | | | | | |
| AEs leading to study | 0 | | 0 | | 0 | |
| discontinuation | | | | | | |
| AEs leading to death | 0 | | 0 | | 0 | |
| Gastrointestinal TEAEs | | | | | | |
| Diarrhoea | 6 | (54.5) | 1 | (9.1) | 1 | (33.3) |

| | | | | | |
|---|---|---|---|---|---|
| Abdominal pain | 5 | (45.5) | 2 | (18.2) | 0 |

^{a}Subjects were counted only once for each preferred term per period.
^{b}Before age 16 years is defined as the last data point prior to turning 16 years of age. After age 16 years is defined as the first data point after turning 16 years of age.
^{b}The treatment-related AE ≥ grade 3 was increased pruritus (grade 3) that was resolved without dose modification.

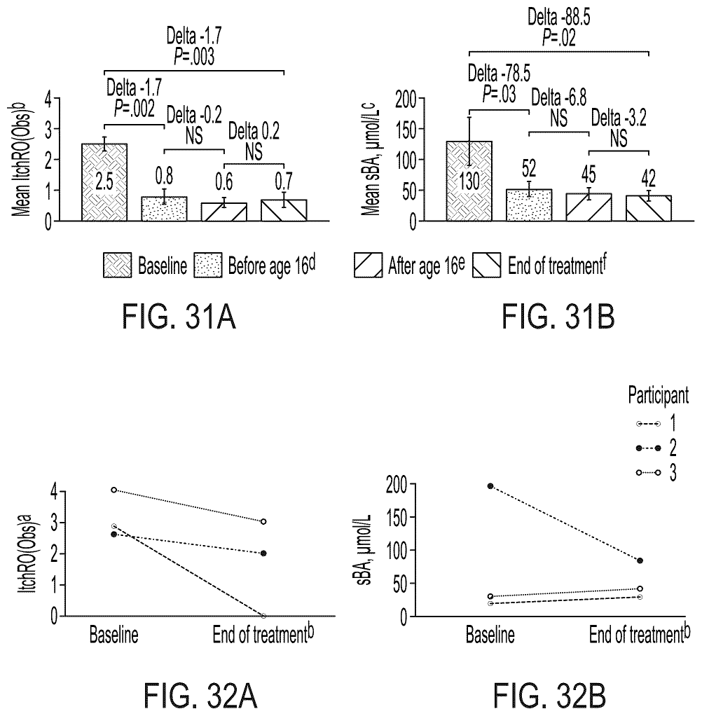

Patients receiving MRX had significant improvements in pruritus and sBA during childhood that were maintained into early adulthood (FIGS. 31A-B and 32A-B). Participants receiving MRX in early adulthood showed significant improvements in pruritus and sBA upon treatment, which persisted throughout therapy. MRX was generally well tolerated and demonstrated a safety and tolerability profile consistent with data reported previously.

These results provide critical data for patients who transition to adulthood while on MRX therapy. MRX showed the potential for a positive impact on the management of adults with ALGS who survive with their native livers into adulthood.

### Example 11. Maralixibat Leads to Significant Reductions in Bilirubin for Patients with Progressive Familial Intrahepatic Cholestasis (PFIC)

Methods: MARCH enrolled patients with a genetic diagnosis of PFIC, pruritus, and elevated sBA. Patients were randomized to MRX 57011 g/kg BID or placebo (PBO) for 26 weeks. Changes were determined as the difference between MRX and PBO groups for Change from Baseline (CFB) to the average of the final 3 measurements (Weeks 18, 22 and 26) using a mixed-effects model with recurrent measurements. Total/direct bilirubin (TB/DB) categories were defined as normal (≤1.2/0.3 mg/dL) or abnormal (>1.2/0.3 mg/dL).

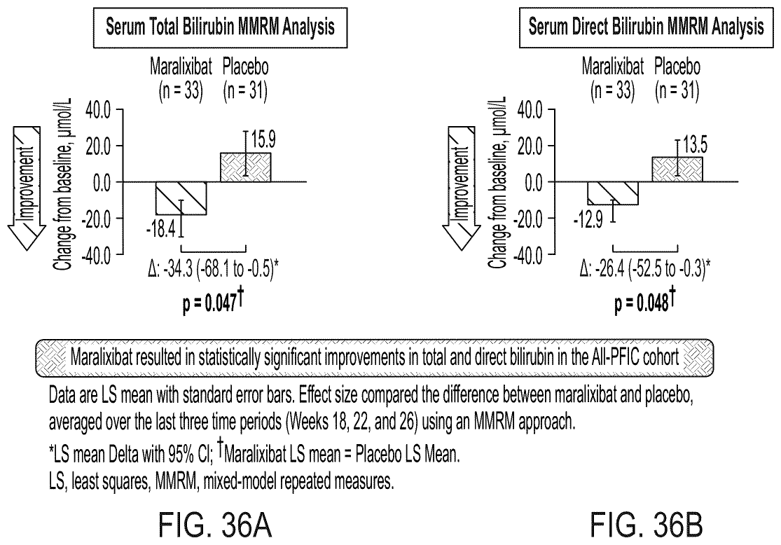

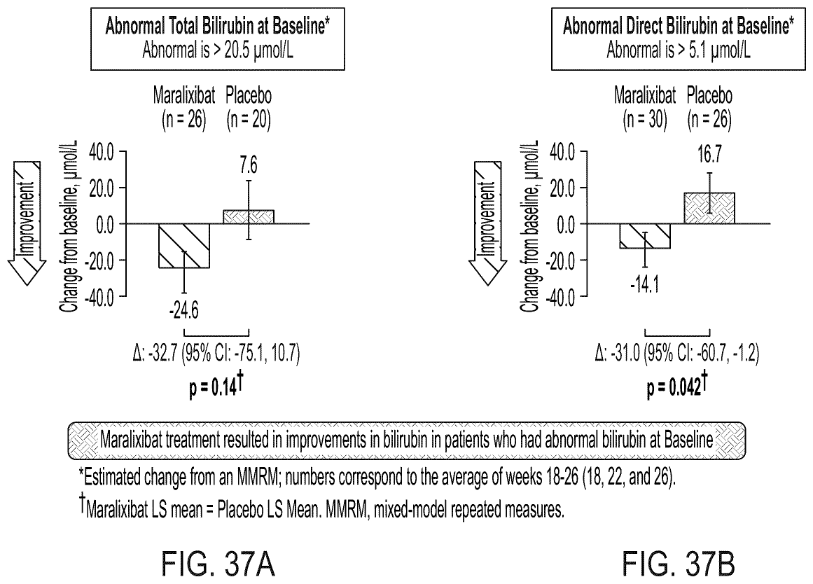

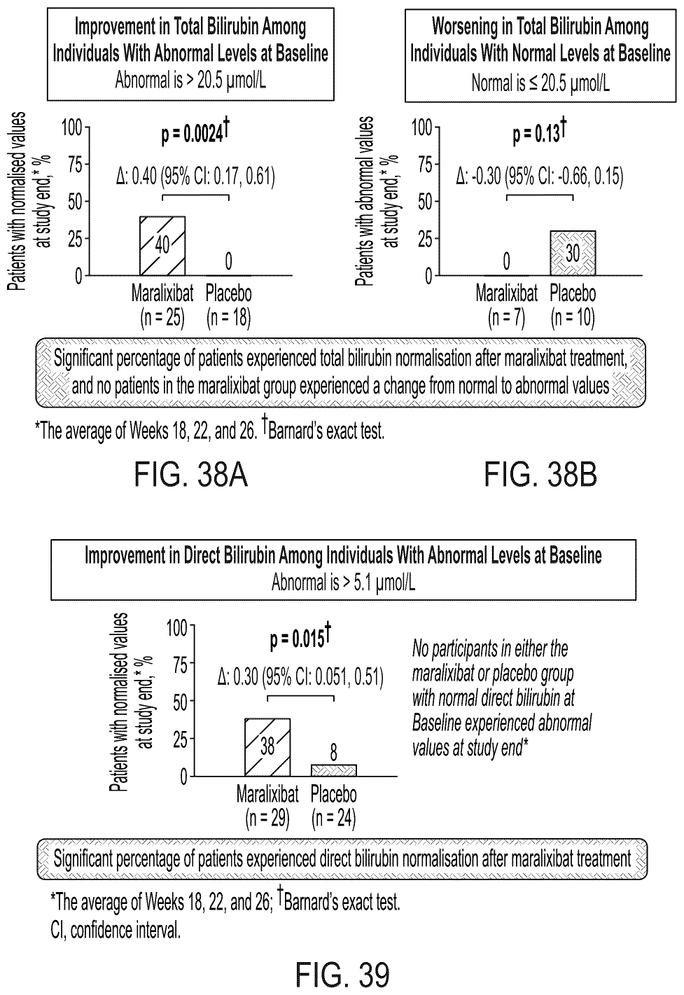

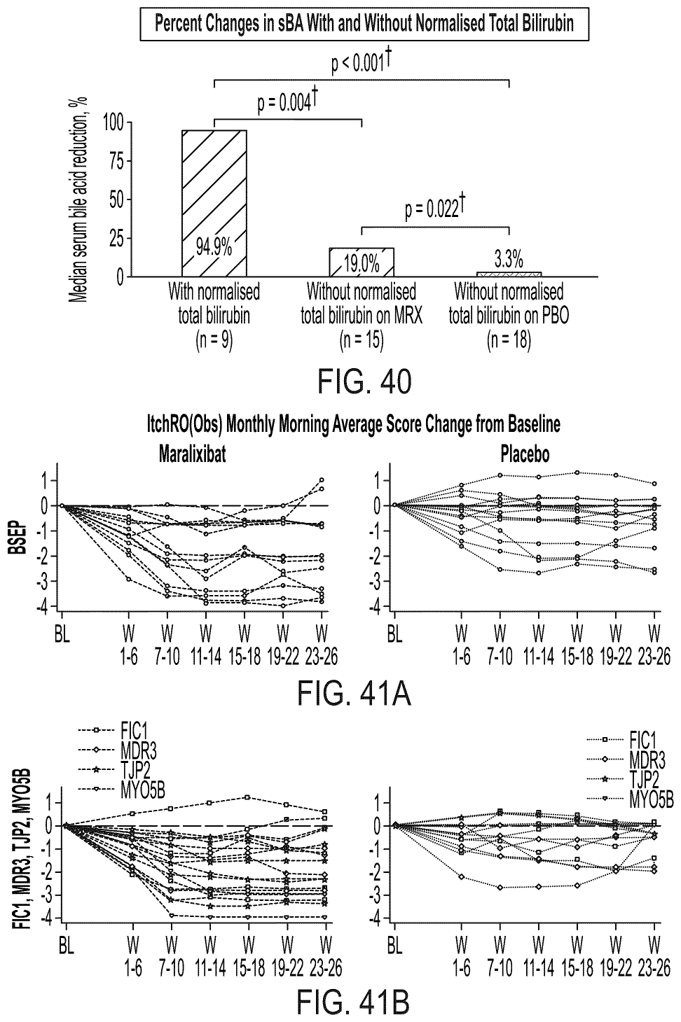

Results: Analysis included 64 patients from the All-PFIC cohort (13 FIC1, 31 nt-BSEP, 9 MDR3, 7 TJP2, and 4MYO5B); patients were randomized to MRX (n=33) or PBO (n=31). Baseline median (Q1, Q3) TB in the MRX and PBO groups were 2.8 (1.4, 5.5) and 2.6 (0.8, 5.5) mg/dL, and DB were 2.1 (0.9, 4.0) mg/dL and 1.9 (0.5, 4.3) mg/dL, respectively. The study achieved significant CFB between MRX vs PBO for TB (−1.1 vs+0.9 mg/dL; group difference: 2.0; p=0.047) (FIG. 36A) and DB (−0.8 vs+0.8 mg/dL; group difference: 1.5; p=0.048) (FIG. 36B). Among individuals with abnormal Baseline bilirubin (TB: n=46; DB: n=56), there was a significant CFB between MRX vs PBO groups for DB (−0.8 vs+1.0 mg/dL; group difference: −1.8; p=0.042) (FIGS. 37A and 37B). For the post-hoc analysis assessing bilirubin normalization, pre and post bilirubin values were available for 60 individuals (32 MRX; 28 PBO). For the MRX group, TB normalized in 40% (10/25) of patients with abnormal Baseline values, and no patient went from normal to abnormal TB (0/7) (FIGS. 38A and 38B). For the PBO group, TB never normalized in those with abnormal Baseline values (0/18) and instead became abnormal for 30% (3/10) of patients (FIGS. 38A and 38B). For DB, the MRX group also showed greater frequency of normalization vs PBO (38% vs 8%) (FIG. 39). Among all individuals that normalized TB, sBA was reduced by 94.9% (95% CI: 68.5%, 98.9%) whereas for those individuals that did not normalize TB, sBA only decreased by 13.3% (95% CI: 1.4, 34.0); p<0.0001 (FIG. 40). For treatment-emergent adverse-event reporting, bilirubin increases were observed less frequently in MRX vs PBO (14.9% vs 19.6%).

Conclusions: MRX is the only MAT inhibitor to demonstrate significant decreases in TB/DB compared to PBO in children with PFIC and across PFIC types. 40% of MRX patients with abnormal Baseline bilirubin achieved normalization vs none in the PBO group, suggesting that MRX may yield clinically meaningful improvements in liver health in patients with PFIC. Reductions in bilirubin corresponded with reductions in sBA.

### Example 12. Maralixibat Leads to Surprising and Unexpected Improvements in Truncated BSEP (t-BSEP) Patients

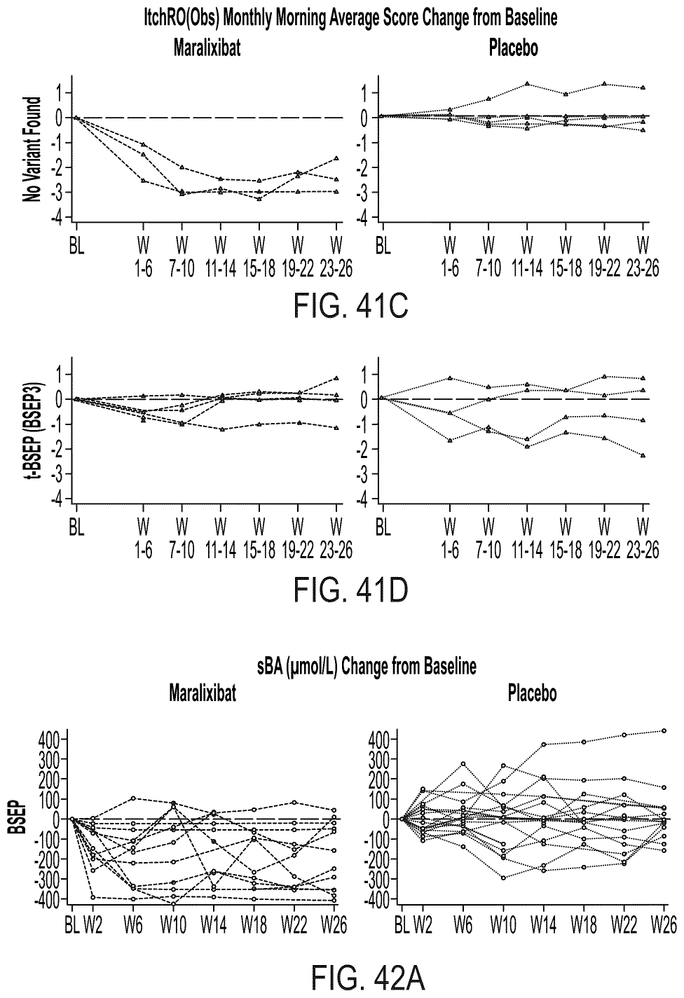

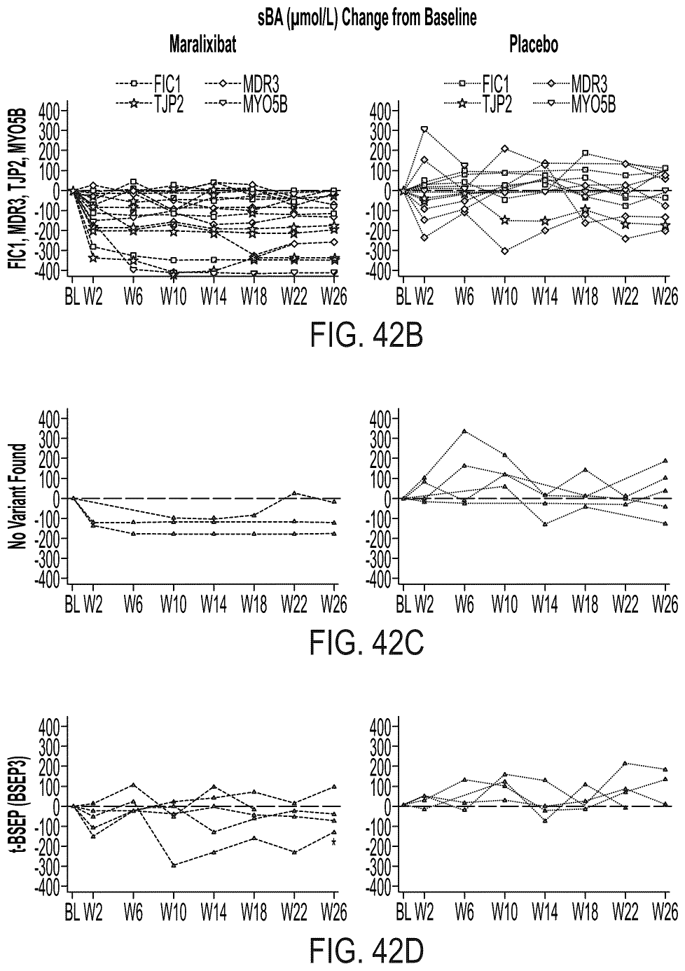

An unexpected finding was observed in patients with a truncated BSEP (t-BSEP) deficiency, also referred to as truncated PFIC2, or t-PFIC2, where improvements were seen in individual patients, with reductions from Baseline to Week 26 in sBA and limited improvement in pruritus (FIGS. 41D and 42D). A portion of t-B SEP patients experienced a reduction in pruritus as shown in FIG. 41D, where t-BSEP patients reported a reduction in morning pruritus severity over time as measure by Itch-Reported Outcome.

Table 15, below, shows data for individual t-BSEP patients. All patients receiving maralixibat (MRX-MRX), or switching to maralixibat from placebo (PBO-MRX), show a meaningful sBA reduction.

TABLE 15
Summary of t-BSEP patients' sBA, total bilirubin and ALT at week 26 and week 58 of treatment

| | | | sBA (μmol/L) | | | Total Bilirubin (mg/dL) | | | ALT (U/L) | | |
|---|---|---|---|---|---|---|---|---|---|---|---|
| Participant | | | | Chg | % Chg | | Chg | % Chg | | Chg | % Chg |
| ID | Treatment | Timepoint | Value | from BL | from BL | Value | from BL | from BL | Value | from BL | from BL |
| 008-001 | MRX-MRX | Baseline | 265 | | | 1.15 | | | 123 | | |
| | | Week 26 | 364 | +99 | +37.4% | 0.9 | −0.25 | −21.7% | 118 | 5 | −4.1% |
| | | Week 58 | 208 | −57 | 21.5% | 0.8 | −0.35 | −30.4% | 116 | −7 | −5.7% |
| 033-006 | MRX-MRX | Baseline | 516 | | | 3.85 | | | 320 | | |
| | | Week 26 | 388 | −128 | −24.8% | 2.6 | −1.25 | −32.5% | 183 | −127 | −42.8% |
| | | Week 58 | 48.6 | −467.4 | −90.6% | 0.9 | −2.95 | −76.6% | 28 | −292 | −91.3% |
| 033-010 | MRX-MRX | Baseline | 453 | | | 2.25 | | | 381 | | |
| | | Week 26 | 408 | −45 | −9.9% | 3.10 | +0.85 | +37.8% | 439 | +58 | +15.2% |
| | | Week 58 | N/A | N/A | N/A | N/A | N/A | N/A | N/A | N/A | N/A |
| 038-003 | MRX-MRX | Baseline | 389 | | | 0.9 | | | 43 | | |
| | | Week 26 | 314 | −75 | −19.3% | 0.7 | −0.2 | −22.2% | 44 | +1 | +2.3% |
| | | Week 58 | N/A | N/A | N/A | N/A | N/A | N/A | N/A | N/A | N/A |
| 016-003 | PBO-MRX | Baseline | 410 | | | 3.95 | | | 297 | | |
| | | Week 26 | 588 | +178 | +43.4% | 5.3 | +1.35 | +34.2% | 254 | −43 | −14.5% |
| | | Week 52[a] | 222 | −366 | −62.2% | 1.2 | −4.1 | −77.4% | 195 | −59 | −23.2% |
| 023-002 | PBO-MRX | Baseline | 157 | | | 1.1 | | | 76 | | |
| | | Week 26 | 282 | +125 | +79.6% | 1.4 | +0.3 | +27.3% | 92 | +16 | +21.1% |
| | | Week 52[a] | 349 | +67 | +23.8% | 1.0 | −0.4 | −28.6% | 170 | +78 | +84.8% |
| 035-004 | PBO-MRX | Baseline | 234 | | | 3.25 | | | 136 | | |
| | | Week 26 | 236 | +2 | +0.9% | 5.0 | +1.75 | +53.8% | 87 | −49 | −36.0% |
| | | Week 52[a] | 216 | −20 | −8.5% | 7.7 | +2.7 | +54.0% | 274 | +187 | +214.9% |
| 043-001 | PBO-MRX | Baseline | 434 | | | 2.65 | | | 323 | | |
| | | Week 26[b] | 42/ | −7 | −1.6% | 1.55 | −1.1 | −41.5% | 234 | −89 | −27.6% |
| | | Week 52[a,c] | 309 | −118 | −27.6% | 1.7 | +0.15 | +9.7% | 345 | +111 | +47 4% |

[a]Week 52 values are Week 26 values from study MRX-503 (representing 26 weeks of MRX treatment). The (present) change from baseline is calculated relative to the Week 26 result from study MRX-502.
[b]For Participant 043-001, Week 26 results were not available so the baseline value from MRX-503 was used instead.
[c]Week 22 results from Study MRX-503 were used.

This finding is highly surprising and unexpected given the predicted loss of function associated with this genotype.

All references cited anywhere within this specification are incorporated herein by reference in their entirety for all purposes.

While preferred embodiments of the present invention have been shown and described herein, it will be obvious to those skilled in the art that such embodiments are provided by way of example only.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

Al Mehaidib A, Al Shahrani A. 1381 Progressive familial intrahepatic cholestasis in Arabs. J Hepatol 2013; 58:5555-6.

Alissa F T, Jaffe R, Shneider B L. Update on progressive familial intrahepatic cholestasis. J Pediatr Gastroenterol Nutr 2008; 46:241-52.
Arnell H, Papadogiannakis N, Zemack H, et al. Follow-up in children with progressive familial intrahepatic cholestasis after partial external biliary diversion. J Pediatr Gastroenterol Nutr 2010; 51:494-9.
Cnaan A, Laird N M, Slasor P. Using the general linear mixed model to analyse unbalanced repeated measures and longitudinal data. Stat Med 1997; 16, 2349-80.
Davit-Spraul A, Gonzales E, Baussan C, et al. Progressive familial intrahepatic cholestasis. Orphanet J Rare Dis 2009; 4:1.
Emerick K M, Elias M S, Melin-Aldana H, et al. Bile composition in Alagille syndrome and PFIC patients having partial external biliary diversion. BMC Gastroenterol 2008; 8:47.
Ethical Considerations for Clinical Trials on Medicinal Products Conducted with Minors. 2017. Accessed at: https://ec.europa.eu/health/sites/health/files/files/eudralex/vol-10/2017_09_18 ethical_consid_ct_with_minors.pdf
European Medicines Agency. 2010. Guideline on missing data in confirmatory clinical trials.
Halaweish I, Chwals W J. Long-term outcome after partial external biliary diversion for progressive familial intrahepatic cholestasis. J Pediatr Surg 2010; 45:934-7.
Hounnou G, Destrieux C, Desme J, et al. Anatomical study of the length of the human intestine. Surg Radiol Anat 2002; 24:290-4.
Jericho H. Bile acid pool dynamics in progressive familial intrahepatic cholestasis with partial external bile diversion. J Pediatr Gastroenterol Nutr 2015; 60:368-74.
Kenward M G, Roger J H. Small sample inference for fixed effects from restricted maximum likelihood. Biometrics 1997; 53:983-97.
Little R, Rubin D. Statistical analysis with missing data. New York: John Wiley: 1987.
Little R, Yau L. Intent-to-treat analysis for longitudinal studies with drop-outs, Biometrics 1996; 52:1324-33.
Mallinckrodt C H, Clark W S, David S R. Accounting for dropout bias using mixed-effects models. J Biopharm Stat 2001; 11:9-21.
Mallinckrodt C H, Lane P W, Schnell D, et al. Recommendations for the primary analysis of continuous endpoints in longitudinal clinical trials. Drug Information Journal 2008; 42:303-19.
Mallinckrodt C H, Roger J, Chuang-Stein C, et al. Recent developments in the prevention and treatment of missing data. Drug Information Journal 2013; 48:68-80.
Molenberghs G, Kenward M G. Missing data in clinical studies. New York: Wiley:2007.
Molenberghs G, Thijs H, Jansen I, et al. Analyzing incomplete longitudinal clinical trial data. Biostatistics 2004; 5:445-64.
Pawlikowska L, Strautnieks S, Jankowska I, et al. Differences in presentation and progression between severe FIC1 and BSEP deficiencies. J Hepatol 2010; 53:170-8.
Ratitch B, O'Kelly M. 2011. Implementation of pattern-mixture models using standard SAS/STAT procedures. Proceedings of PharmaSUG 2011 (Pharmaceutical Industry SAS Users Group), SP04, Nashville.
Sambrotta M. Mutations in TJP2 cause progressive cholestatic liver disease. Nat Genet 2014; 46:326-8.
Schafer J L. Analysis of incomplete multivariate data. New York: Chapman & Hall:1997.
Schukfeh N, Metzelder M L, Petersen C, et al. Normalization of serum bile acids after partial external biliary diversion indicates an excellent long-term outcome in children with progressive familial intrahepatic cholestasis. J Pediatr Surg 2012; 47:501-5.
Shneider B L, Magee J C, Bezerra J A, et al. Efficacy of fat-soluble vitamin supplementation in infants with biliary atresia. Pediatrics 2012; 130:607-14.
Siddiqui O, Hung H M, O'Neill R. MMRM vs. LOCF: a comprehensive comparison based on simulation study and 25 NDA datasets. J Biopharm Stat 2009; 19:227-46.
Stapelbroek J M, van Erpecum K J, Klomp L W J, et al. Liver disease associated with canalicular transport defects: current and future therapies. J Hepatol 2010; 52:258-71.
Varni J W, Seid M, Kurtin P S. PedsQL 4.0: reliability and validity of the Pediatric Quality of Life Inventory version 4.0 generic core scales in healthy and patient populations. Medical Care 2001; 39:800-12.
Verbeke G, Molenberghs G. Linear mixed models for longitudinal data. New York: Springer: 2000.
Weaver L T, Austin S, Cole T J. Small intestinal length: a factor essential for gut adaptation. Gut 1991; 32:1321-3.
Whitington P F, Whitington G L. Partial external diversion of bile for the treatment of intractable pruritis associated with intrahepatic cholestasis. Gastroenterology 1988; 95:130-6.
Yang H, Porte R J, Verkade H J, et al. Partial external biliary diversion in children with progressive familial intrahepatic cholestasis and Alagille disease. J Pediatr Gastroenterol Nutr 2009; 49:216-21.

1. A method for treating a progressive familial intrahepatic cholestasis (PFIC) in a subject in need thereof comprising administering to the subject maralixibat, or a pharmaceutically acceptable salt thereof, wherein the maralixibat or pharmaceutically acceptable salt thereof is administered in an amount from about 600 μg/kg/day to about 1200 μg/kg/day.

2. The method of claim 1, wherein the pharmaceutically acceptable salt of maralixibat is maralixibat chloride, maralixibat bromide, maralixibat acetate, or maralixibat mesylate.

3. The method of claim 1, wherein the pharmaceutically acceptable salt of maralixibat is maralixibat chloride.

4.-6. (canceled)

7. The method of claim 1, wherein the maralixibat or pharmaceutically acceptable salt thereof is maralixibat chloride, and maralixibat chloride is administered in an amount of about 1200 μg/kg/day.

8. (canceled)

9. The method of claim 1, wherein the PFIC is PFIC 1, PFIC 2, PFIC 3, PFIC 4, PFIC 5, or PFIC 6.

10.-17. (canceled)

18. The method of claim 1, wherein the PFIC is heterozygous.

19. The method of claim 1, wherein the subject has intermittent cholestasis and/or has undergone biliary diversion surgery.

20. (canceled)

21. The method of claim 1, wherein the subject is a pediatric subject.

22. (canceled)

23. (canceled)

24. The method of claim 1, wherein the subject has a mutation in a gene selected from the group consisting of: ATP8B1, ABCB11, ABCB4, TJP2, NR1H4, and MYO5B.

25. The method of claim 24, wherein the mutation is selected from a non-truncating mutation and a truncating mutation.

26. (canceled)

27. The method of claim 1, wherein the subject has a truncated B SEP protein.

28. (canceled)

29. The method of claim 1, wherein the maralixibat or pharmaceutically acceptable salt thereof is administered twice daily (BID).

30. The method of claim 1, wherein the maralixibat or pharmaceutically acceptable salt thereof is maralixibat chloride, and maralixibat chloride is administered at 600 μg/kg/day BID for a total daily dose of 1200 μg/kg/day.

31.-36. (canceled)

37. The method of claim 1, wherein the administration of the maralixibat reduces intensity of pruritus as measured by an ItchRO(Obs) score or a CSS score by at least 1.0 points relative to baseline.

38.-46. (canceled)

47. The method of claim 1, wherein the administration of the maralixibat or pharmaceutically acceptable salt thereof improves sleep as measured by a reduction of an EDQ(Obs) or EDQ(Pt) score of the subject by at least 1.0 points relative to baseline.

48.-53. (canceled)

54. The method of claim 1, wherein the administration of the maralixibat or pharmaceutically acceptable salt thereof reduces total bilirubin and/or direct bilirubin by at least 0.2 mg/dL relative to baseline.

55.-66. (canceled)

67. The method of claim 1, wherein the maralixibat or pharmaceutically acceptable salt thereof is administered BID, about 30 minutes before the morning meal and about 30 minutes before the evening meal.

68. The method of claim 1, wherein the maralixibat is administered in the form of a pharmaceutical composition comprising maralixibat, or a pharmaceutically acceptable salt thereof, an antioxidant, and a preservative.

69. The method of claim 68, wherein the pharmaceutical composition is a liquid composition for oral administration.

70.-74. (canceled)

75. The method of claim 1, wherein the preservative is an antimicrobial preservative selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxy ethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

76.-80. (canceled)

81. The method of claim 68, wherein the antioxidant is an aminopolycarboxylic acid selected from ED TA (ethylene diaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid).

82.-84. (canceled)

85. The method of claim 68, wherein the pharmaceutical composition comprises:
a. from about 8 mg/mL to about 20 mg/mL of maralixibat;
b. from about 330 mg/mL to about 380 mg/mL of propylene glycol;
c. about 1 mg/mL of disodium EDTA;
d. a sweetener, a taste-masking ingredient, or a combination thereof, and
e. water.
